(19) United States
(12) Patent Application Publication
(10) Pub. No.: US 2026/0207728 A1
(43) Pub. Date: Jul. 23, 2026

(54) IMMUNOGENIC EBOLAVIRUS FUSION PROTEINS AND RELATED METHODS

(71) Applicants: CZ Biohub SF, LLC, San Francisco, CA (US); The Board of Trustees of the Leland Stanford Junior University, Stanford, CA (US)

(72) Inventors: Duo Xu, Madison, WI (US); Abigail E. Powell, Sunnyvale, CA (US); Peter S. Kim, Stanford, CA (US)

(73) Assignees: CZ Biohub SF, LLC, San Francisco, CA (US); The Board of Trustees of the Leland Stanford Junior University, Stanford, CA (US)

(21) Appl. No.: 19/139,519

(22) PCT Filed: Dec. 15, 2023
(86) PCT No.: PCT/US2023/084184

§ 371 Date: Jun. 16, 2025

Related U.S. Application Data
(60) Provisional application No. 63/433,067, filed on Dec. 16, 2022.

(51) Int. Cl. A61K 39/12; A61K 39/00; A61P 31/14; C07K 14/005; C07K 14/435; C12N 7/00; C12N 15/85

(52) U.S. Cl. CPC A61K 39/12; A61P 31/14; C07K 14/005; C07K 14/435; C12N 7/00; C12N 15/85; A61K 2039/55555; A61K 2039/575; C07K 2319/91; C12N 2760/14122; C12N 2760/14134

(57) ABSTRACT

Provided are fusion proteins including an artificially modified amino acid sequence of an ebolavirus glycoprotein (GP) joined to an amino acid sequence of a ferritin subunit polypeptide. An artificially modified amino acid sequence of an ebolavirus GP includes artificially installed glycosylation sites. Nanoparticles including fusion proteins, with surface-exposed trimers of the ebolavirus GP, are also provided. Also provided are nucleic acids and vectors encoding the fusion proteins, cells containing such nucleic acid and vectors, immunogenic compositions including the fusion proteins, the nanoparticles, or the vectors, as well as corresponding methods and kits.

### CROSS-REFERENCE TO RELATED APPLICATIONS

This application claims the benefit of priority to U.S. Provisional Patent Application No. 63/433,067, filed Dec. 16, 2022, the entire contents of which are incorporated by reference herein.

### STATEMENT AS TO RIGHTS TO INVENTIONS MADE UNDER FEDERALLY SPONSORED RESEARCH AND DEVELOPMENT

This invention was made with Government support under Director's Pioneer Award DP1-AI158125 awarded by the National Institutes of Health. The Government has certain rights in the invention.

### BACKGROUND

Ebola virus, a member of the Filoviridae family, is highly pathogenic and causes hemorrhagic fever in humans with high mortality (Malvy et al. 2019). Since its discovery in 1976, Ebola virus has led to more than 20 outbreaks in Africa, most notably the 2014-2016 epidemic that quickly became an international public health emergency (Malvy et al. 2019; Weyer et al. 2015; Jacob et al. 2020). Two viral vector vaccines (Ervebo and Zabdeno/Mvabea) have been approved for the prevention of Ebola virus disease (Feldmann et al. 2020). Nonetheless, neither vaccine is widely distributed to prevent outbreaks but has only been used in limited “ring vaccination” settings to protect high-risk groups throughout endemic areas during active outbreaks (Kucharski et al. 2016; Henao-Restrepo et al. 2017). Both viral vector vaccines also require cold-chain storage (Woolsey et al. 2021) and may cause mild to moderate side effects in vaccinated individuals (Regules et al. 2017; Suder et al. 2018). In addition, these two vaccines only prevent infection from Zaire ebolavirus (EBOV) (FDA News Release 2019; European Medicines Agency, Zabdeno), whereas three species of the ebolavirus genus have caused large outbreaks and remain an ongoing threat: EBOV, Bundibugyo ebolavirus (BDBV) and Sudan ebolavirus (SUDV) (Weyer et al. 2015; Jacob et al. 2020). Long-term control of Ebola virus disease will require vaccines that are suitable for widespread use and confer durable and cross-protective immunity against different Ebola species.

### SUMMARY

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the exemplary embodiments of the present invention are discussed below.

Included among the embodiments of the present invention and described in the present disclosure are fusion proteins of an artificially modified amino acid sequence of an ebolavirus glycoprotein (GP) and an amino acid sequence of a ferritin subunit polypeptide. In some embodiments, the artificially modified amino acid sequence of the ebolavirus GP included in a fusion protein is a sequence with at least 90% sequence identity to SEQ ID NO:13, SEQ ID NO:14, or SEQ ID NO:15. In some embodiments, an artificially modified amino acid sequence of an ebolavirus GP is derived from Zaire ebolavirus. In some embodiments, the artificially modified amino acid sequence of the ebolavirus GP included in a fusion protein comprises a deletion of the mucin-like domain. In some embodiments, the artificially modified amino acid sequence of the ebolavirus GP included in a fusion protein comprises at least two artificial glycosylation sites. In some embodiments, the artificially modified amino acid sequence of the ebolavirus GP included in a fusion protein comprises two, three, or four artificial glycosylation sites. In some embodiments, the artificial glycosylation sites are located in the glycan cap domain of the ebolavirus GP. In some embodiments, the artificially modified amino acid sequence of the ebolavirus GP included in a fusion protein contains two or more amino acid substitutions at positions corresponding to positions 251, 280, 272, 274, 212, and 214 of SEQ ID NO:12, wherein the amino acid substitutions result in the artificial glycosylation sites. In some embodiments, the two or more amino acid substitutions are D251N, L280N, S272N, E274T, Q212N, and E214T. In some embodiments, the ferritin subunit polypeptide of the fusion protein is Helicobacter pylori ferritin subunit polypeptide. In some embodiments, the amino acid sequence of the ferritin subunit polypeptide is a sequence with at least 90% sequence identity to SEQ ID NO:7. In some embodiments, the ferritin subunit polypeptide contains one or more artificial glycosylation sites. In some embodiments of a fusion protein, the artificially modified amino acid sequence of the ebolavirus GP is joined to the amino acid sequence of the ferritin subunit polypeptide by a linker amino acid sequence. In some embodiments of a fusion protein, the artificially modified amino acid sequence of the ebolavirus GP is preceded by an amino acid sequence of a signal peptide. In some embodiments of a fusion protein, the amino acid sequence of the signal peptide is MGVTGILQLPRDRFKRTSFFLWVIILFQRTF (SEQ ID NO:20). In some embodiments, the amino acid sequence of the fusion protein is a sequence with at least 90% sequence identity to SEQ ID NO:9, SEQ ID NO:10, or SEQ ID NO:1.

Included among the embodiments of the present invention and described in the present disclosure are nanoparticles comprising an oligomer of the fusion protein according to the present disclosure. In some embodiments, the nanoparticle comprises surface-exposed trimers of the ebolavirus GP. In some embodiments, the nanoparticle comprises eight of the surface-exposed trimers of the ebolavirus GP. Also included among the embodiments of the present invention and described in the present disclosure are nucleic acids encoding one or more fusion proteins according to the present disclosure. In some embodiments, the nucleic acid is DNA or RNA. Also included among the embodiments of the present invention and described in the present disclosure are vectors comprising the nucleic acids according to the present disclosure. Also included among the embodiments of the present invention and described in the present disclosure are cells comprising one or more nucleic acids according to the present disclosure or one or more vectors according to the present disclosure. In some embodiments, the cell is a mammalian cell. In some embodiments, the cell comprises one or more nucleic acids according to the present disclosure, wherein one or more nucleic acids are stably integrated into genome of the cell in one or more locations. In some embodiments, the cell, which can be a mammalian cell, expresses the fusion protein. Also included among the embodiments of the present invention and described in the present disclosure are cell cultures comprising a plurality of cells according to the present disclosure.

Included among the embodiments of the present invention and described in the present disclosure are immunogenic compositions comprising one or more fusion proteins according to the present disclosure, one or more nanoparticles according to the present disclosure, one or more nucleic acids according to the present disclosure, or one or more vectors according to the present disclosure. In some embodiments, an immunogenic composition comprises two or more different fusion proteins according to the present disclosure, two or more different nanoparticles according to the present disclosure, two or more different nucleic acids according to the present disclosure, or two or more different vectors according to the present disclosure. In some embodiments, an immunogenic composition according to the present disclosure comprises one or more adjuvants. In some embodiments, an immunogenic composition is lyophilized. Also included among the embodiments of the present invention and described in the present disclosure are kits comprising one or more immunogenic compositions according to the present disclosure and one or more of: a device for administering the immunogenic composition, and an excipient.

Included among the embodiments of the present invention and described in the present disclosure are methods of inducing an immune response in a subject, the methods comprising a step of administering to the subject an immunogenic composition according to the present disclosure. In some embodiments, an immunogenic composition is administered in an amount capable of eliciting a protective immune response against an ebolavirus in the subject. In some embodiments, the protective immune response is against one or more of Zaire ebolavirus, Bundibugyo ebolavirus, and Sudan ebolavirus. In some embodiments, the protective immune response against the ebolavirus comprises production of neutralizing antibodies against the ebolavirus in the subject. In some embodiments, the subject is a human. Also included among the embodiments of the present invention and described in the present disclosure are methods of producing fusion proteins according to the present disclosure, the methods comprising the steps of: introducing into a cell one or more nucleic acids according to the present disclosure or one or more vectors according to the present disclosure; incubating the cell under conditions allowing for expression of a fusion protein according to the present disclosure; and isolating the fusion protein. In some embodiments, the step of introducing comprises stably integrating the nucleic acid into genome of the cell. In some embodiments of the above methods, the cell is a mammalian cell. Also included among the embodiments of the present invention and described in the present disclosure are methods of producing nanoparticles according to the present disclosure, the methods comprising the steps of: introducing into a cell one or more nucleic acids according to the present disclosure or one or more vectors according to the present disclosure; incubating the cell under conditions allowing for expression of the fusion protein and self-assembly of the nanoparticle; and isolating the nanoparticle. In some embodiments of the above methods, the introducing comprises stably integrating the nucleic acid into genome of the cell. In some embodiments of the above methods, the cell is a mammalian cell.

### BRIEF DESCRIPTION OF THE DRAWINGS

FIG. 1 is a bar graph illustrating the analysis of expression levels of GP with single-glycan mutations according to the present disclosure by Western blotting. The expression levels of GP variants were normalized to the expression of wild-type GP. Dashed line indicates 50% of GP expression and arrows indicate GP glycosylation variants with >50% GP expression, as compared to the expression of wild-type GP. Data are presented as mean±standard deviation of 4 replicates.

FIG. 2 is a schematic illustration of the structure of GP+2, GP+3, and GP+4 variants according to aspects of the present disclosure. The locations of glycan residues are indicated by arrows.

FIG. 3 is a dot plot illustrating melting temperatures, measured by differential scanning fluorimetry, of GP and of GP+2, GP+3, and GP+4 variants according to aspects of the present disclosure. The plotted values represent 3 replicates.

FIG. 4 is a summary of analysis of GP and of GP+2, GP+3, and GP+4 variants according to aspects of the present disclosure with six GP-specific mAbs by ELISA. The numbers represent fold change in the binding affinity (EC50) of GP-specific mAbs to GP, GP+2, GP+3, and GP+4. EC50 (antibody concentration with half-maximal binding) was calculated from ELISA curves and normalized to values obtained for GP.

FIG. 5 is a dot plot illustrating melting temperatures, as measured by differential scanning fluorimetry, of GP-Fer, GP+2-Fer, GP+3-Fer, and GP+4-Fer according to aspects of the present disclosure. The plotted values represent the mean of 3 replicates.

FIG. 6 is a summary of analysis of GP-Fer, GP+2-Fer, GP+3-Fer, and GP+4-Fer ferritin nanoparticles according to aspects of the present disclosure with six GP-specific mAbs by ELISA. Fold change in the binding affinity (EC50) of GP-specific mAbs to GP+2-Fer, GP+3-Fer, and GP+4-Fer was normalized to values obtained from the GP-Fer.

FIG. 7 are representative transmission electron microscopy (TEM) images of GP-Fer, GP+2-Fer, GP+3-Fer, and GP+4-Fer ferritin nanoparticles according to aspects of the present disclosure. The arrows indicate representative ferritin nanoparticles. The scale bar is 100 nm.

FIG. 8 are plots illustrating the results of mouse immunization with GP (open circles) or GP-Fer (according to aspects of the present disclosure, closed circles) using a prime-boost regimen on day 0 and 21. Ten mice per group were immunized with 5 μg of antigens adjuvanted with MPLA/Quil®-A (10 μg/10 μg) via subcutaneous injections. Line plot illustrating EBOV GP-specific IgG titers over time is shown on the left. Dot plot illustrating EBOV GP-specific IgG titers of individual mice on day 42 (post-boost) is shown on the right. Dashed lines indicate the limit of quantification. The data are presented as geometric mean±standard deviation of log-transformed data. Comparisons of IgG titers or NT₅₀ over time were performed using a two-way ANOVA followed by a Bonferroni test. Comparisons of two groups were performed using the two-tailed Mann-Whitney U test. P values of 0.05 or less were considered significant and plotted.

FIG. 9 are plots illustrating the results of mouse immunization with GP (open circles) or GP-Fer (ferritin nanoparticles according to aspects of the present disclosure, closed circles) using a prime-boost regimen on day 0 and 21. Ten mice per group were immunized with 5 μg of antigens adjuvanted with MPLA/Quil®-A (10 μg/10 μg) via subcutaneous injections. Line plot illustrating neutralization titers (NT₅₀—the serum dilution required to neutralize 50% of EBOV GP-pseudotyped lentiviruses) against EBOV over time is shown on the left. Dot plot illustrating NT₅₀ against EBOV from individual mice on day 42 (post-boost) is shown on the right. Dashed lines indicate the limit of quantification. The data are presented as geometric mean±standard deviation of log-transformed data. Comparisons of IgG titers or NT₅₀ over time were performed using a two-way ANOVA followed by a Bonferroni test. Comparisons of two groups were performed using the two-tailed Mann-Whitney U test. P values of 0.05 or less were considered significant and plotted.

FIG. 10 are plots illustrating the results of mouse immunization with GP-Fer, GP+2-Fer, GP+3-Fer, and GP+4-Fer ferritin nanoparticles according to aspects of the present disclosure. Ten mice per group were immunized with 5 μg of antigens adjuvanted with MPLA/Quil®-A (10 μg/10 μg) on day 0, 21 and 42 via subcutaneous injections. Line plot illustrating EBOV GP-specific IgG titers over time is shown on the left. Dot plot illustrating EBOV GP-specific IgG titers of individual mice on day 63 (post-2^{nd} boost) is shown on the right. Dashed lines indicate the limit of quantification. The data are presented as geometric mean±standard deviation of log-transformed data.

FIG. 11 are plots illustrating the results of mouse immunization with GP-Fer, GP+2-Fer, GP+3-Fer, and GP+4-Fer ferritin nanoparticles according to aspects of the present disclosure. Ten mice per group were immunized with 5 μg of antigens adjuvanted with MPLA/Quil®-A (10 μg/10 μg) on day 0, 21 and 42 via subcutaneous injections. Line plot illustrating NT₅₀ against EBOV over time is shown on the left. Dot plot illustrating NT₅₀ against EBOV from individual mice on day 63 (post-2^{nd} boost) is shown on the right. Dashed lines indicate the limit of quantification. The data are presented as geometric mean±standard deviation of log-transformed data.

FIG. 12 are dot plots illustrating cross-reactivity of the endpoint antisera from mice immunized with GP-Fer, GP+2-Fer, GP+3-Fer, or GP+4-Fer ferritin nanoparticles according to aspects of the present disclosure against BDBV GP (left) and SUDV GP (right). Ten mice per group were immunized with 5 μg of antigens adjuvanted with MPLA/Quil®-A (10 μg/10 ug) on day 0, 21 and 42 via subcutaneous injections. The data shown are IgG titers of individual mice on day 63 (post-2nd boost). Dashed lines indicate the limit of quantification. The data are presented as geometric mean±standard deviation of log-transformed data.

FIG. 13 are dot plots illustrating cross-reactivity of the endpoint antisera from mice immunized with GP-Fer, GP+2-Fer, GP+3-Fer, or GP+4-Fer ferritin nanoparticles according to aspects of the present disclosure against BDBV GP-pseudotyped lentivirus (left) or SUDV GP-pseudotyped lentivirus (right) from individual mice on day 63 (post-2nd boost). Ten mice per group were immunized with 5 μg of antigens adjuvanted with MPLA/Quil®-A (10 μg/10 ug) on day 0, 21 and 42 via subcutaneous injections. The pie charts in the right plot indicate the percentage of mice that developed neutralizing activity (NT₅₀ over 20-fold serum dilution) against SUDV GP-pseudotyped lentivirus. Dashed lines indicate the limit of quantification. The data are presented as geometric mean±standard deviation of log-transformed data.

FIG. 14 are plots illustrating the results of mouse immunization with GP-Fer, GP+2-Fer, and GP+3-Fer ferritin nanoparticles according to aspects of the present disclosure. Nine mice per group were immunized with 5 μg of antigens adjuvanted with alum/CpG (500 μg/20 ug) on day 0, 21 and 42 via subcutaneous injections. Line plot illustrating EBOV GP-specific IgG titers over time is shown on the left. Dot plot illustrating EBOV GP-specific IgG titers of individual mice on day 63 (post-2^{nd} boost) is shown on the right. Dashed lines indicate the limit of quantification. The data are presented as geometric mean±standard deviation of log-transformed data.

FIG. 15 are plots illustrating the results of mouse immunization with GP-Fer, GP+2-Fer, and GP+3-Fer ferritin nanoparticles according to aspects of the present disclosure. Nine mice were immunized with 5 μg of antigens adjuvanted with alum/CpG (500 μg/20 μg) on day 0, 21 and 42 via subcutaneous injections. Line plot illustrating NT₅₀ against EBOV over time is shown on the left. Dot plot illustrating NT₅₀ against EBOV from individual mice on day 63 (post-2^{nd} boost) is shown on the right. Dashed lines indicate the limit of quantification. The data are presented as geometric mean±standard deviation of log-transformed data.

FIG. 16 are dot plots illustrating cross-reactivity of the endpoint antisera from mice immunized with GP-Fer, GP+2-Fer, and GP+3-Fer ferritin nanoparticles according to aspects of the present disclosure against BDBV GP (left) and SUDV GP (right). Nine mice were immunized with 5 μg of antigens adjuvanted with alum/CpG (500 μg/20 μg) on day 0, 21 and 42 via subcutaneous injections. The data shown are IgG titers of individual mice on day 63 (post-2nd boost). Dashed lines indicate the limit of quantification. The data are presented as geometric mean±standard deviation of log-transformed data.

FIG. 17 are dot plots illustrating cross-reactivity of the endpoint antisera from the mice immunized with GP-Fer, GP+2-Fer, and GP+3-Fer ferritin nanoparticles according to aspects of the present disclosure against BDBV GP-pseudotyped lentivirus (left) or SUDV GP-pseudotyped lentivirus (right) from individual mice on day 63 (post-2nd boost). Nine mice were immunized with 5 μg of antigens adjuvanted with alum/CpG (500 μg/20 μg) on day 0, 21 and 42 via subcutaneous injections. The pie charts in the right plot indicate the percentage of mice that developed neutralizing activity (NT₅₀ over 20-fold serum dilution) against SUDV GP-pseudotyped lentivirus. Comparisons of means of the treatment groups and the control group were performed using one-way ANOVA with a Dunnett's test. P values of 0.05 or less were considered significant and plotted. Dashed lines indicate the limit of quantification. The data are presented as geometric mean±standard deviation of log-transformed data.

FIG. 18 are plots illustrating the results of mouse immunization GP-Fer, GP+2-Fer, and GP+3-Fer ferritin nanoparticles according to aspects of the present disclosure using a delayed boost regimen. Ten mice per group were immunized with 5 μg of antigens adjuvanted with MPLA/Quil®-A (10 μg/10 μg) on day 0, 42 and 126 via subcutaneous injections. Line plot illustrating EBOV GP-specific IgG titers over time is shown on the left. Dot plot illustrating EBOV GP-specific IgG titers of individual mice on day 133 (post-2nd boost) is shown on the right.

FIG. 19 are plots illustrating the results of mouse immunization GP-Fer, GP+2-Fer, and GP+3-Fer ferritin nanoparticles according to the present disclosure using a delayed boost regimen. Ten mice per group were immunized with 5 μg of antigens adjuvanted with MPLA/Quil®-A (10 μg/10 μg) on day 0, 42 and 126 via subcutaneous injections. Line plot shown to the left illustrate NT₅₀ against EBOV over time. Dot plot shown on the right illustrates NT₅₀ against EBOV from individual mice on day 133 (post-2nd boost). Dashed lines indicate the limit of quantification. The data are presented as geometric mean±standard deviation of log-transformed data.

FIG. 20 are dot plots illustrating the results of mouse immunization GP-Fer, GP+2-Fer, and GP+3-Fer ferritin nanoparticles according to aspects of the present disclosure using a delayed boost regimen. Ten mice per group were immunized with 5 μg of antigens adjuvanted with MPLA/Quil®-A (10 μg/10 μg) on day 0, 42 and 126 via subcutaneous injections. The data plotted are BDBV GP-(left dot plot) or SUDV GP-specific (right dot plot) IgG titers of individual mice on day 133 (post-2nd boost). Comparisons of means of the treatment groups and the control group were performed using one-way ANOVA with a Dunnett's test. P values of 0.05 or less are considered significant and were plotted. Dashed lines indicate the limit of quantification. The data are presented as geometric mean±standard deviation of log-transformed data.

FIG. 21 are dot plots illustrating the results of mouse immunization GP-Fer, GP+2-Fer, and GP+3-Fer ferritin nanoparticles according to aspects of the present disclosure using a delayed boost regimen. Ten mice per group were immunized with 5 μg of antigens adjuvanted with MPLA/Quil®-A (10 μg/10 μg) on day 0, 42 and 126 via subcutaneous injections. The data plotted are NT₅₀ values against BDBV (left) or SUDV (right) from individual mice on day 133 (post-2nd boost). Pie charts in the right plot indicate the percentage of mice that developed neutralizing activity (NT₅₀ over 20-fold serum dilution) against SUDV. Comparisons of means of the treatment groups and the control group were performed using one-way ANOVA with a Dunnett's test. P values of 0.05 or less are considered significant and plotted. Dashed lines indicate the limit of quantification. The data are presented as geometric mean±standard deviation of log-transformed data.

FIG. 22 are dot plots illustrating the results of neutralization of authentic Ebola virus (strain Zaire Mayinga) with pooled mouse antisera from day 63 (3 technical replicates). Substantially similar to the experimental procedures described in reference to FIG. 10, pooled antisera were from mice immunized with GP-Fer, GP+2-Fer, GP+3-Fer, and GP+4-Fer ferritin nanoparticles according to aspects of the present disclosure. Ten mice per group were immunized with 5 μg of antigens adjuvanted with MPLA/Quil®-A (10 μg/10 μg) on day 0, 21 and 42 via subcutaneous injections. Pre-immune and fetal bovine serum (FBS) served as negative controls.

FIG. 23 are dot plots illustrating thermal stability of GP-Fer, GP+2-Fer and GP+3-Fer after incubation at 37° C. for 2, 5, 7, 10, and 14 days. Melting temperatures (Tₘ) were derived from thermal melting curves.

### DETAILED DESCRIPTION

The existence of multiple antigenically distinct Ebolavirus species makes it challenging to design prophylactic vaccines that are suitable for widespread use and confer durable and cross-protective immunity. Combining individual antigens is a direct and common approach to achieving cross-protection (Wang et al. 2021; Hensley et al. 2010; Mire et al. 2013; Ledgerwood et al. 2017; Matassov et al., 2018, Sebastian et al., 2020), but immune responses to heterologous vaccines may bias toward certain immunogens over others, as discussed, for example, in Feldmann et al. 2018. As an improved alternative to heterologous ebolavirus vaccines, the inventors conceived a vaccine based on single protein-based antigen. Such a vaccine is easier to manufacture and allows widespread vaccination strategies that go beyond ring vaccination. Ebola glycoprotein (GP) is the sole Ebolavirus surface protein, and it mediates viral infection of host cells (Lee et al. 2009). GP-based antigens have been shown to elicit mostly ebolavirus species-specific neutralizing antibody responses in animals (Lehrer et al. 2018; Liu et al. 2018; Bengtsson et al. 2016; Konduru et al. 2016; Fan et al. 2019; He et al. 2021; Martinez et al. 2011; Wang et al. 2021).

The inventors discovered that GP-based antigens eliciting antibody responses towards conserved epitopes on GP leads to a cross-neutralizing antibody response towards different Ebolavirus species. Accordingly, the inventors conceived that such antigens may serve as the basis of a universal Ebola vaccine. The inventors utilized hyperglycosylation (as described, for example, in Pantophlet et al. 2003; Lin et al. 2012; Eggink et al. 2014; Duan et al. 2013) to design the antigens based on Ebola GP. Through structure-guided design, the inventors identified locations on the surface of GP to install artificial N-linked glycosylation sites. The inventors then generated GP glycosylation variants with glycans installed in locations masking the poorly conserved glycan cap region. The inventors also created a series of ferritin nanoparticles displaying GP glycosylation variants and tested them in mice. Ferritin nanoparticles were previously used to enhance immunogenicity of protein-based antigens from several pathogens (Kanekiyo et al. 2013; Yassine et al. 2015; Sliepen et al. 2015; He et al. 2016; Powell et al. 2021; Brouwer et al. 2021; Moon et al. 2012), and were described as a promising strategy for generating GP-based vaccines against Ebola (He et al. 2021). However, subsequent studies found that displaying Ebola GP-based antigens on ferritin nanoparticles had minimal or no effect on eliciting neutralizing antibodies in experimental animals following a single-dose immunization (Powell et al. 2022). The inventors surprisingly discovered that a prime-boost immunization in mice with ferritin nanoparticles displaying GP-glycosylation variants described in the present disclosure in some cases, improved the production of anti-GP antibodies. The inventors also discovered that the immunized mice produced antisera that cross-reacted with EBOV, SUDV, and BDBV GP. Mice immunized with ferritin nanoparticles displaying GP-glycosylation variants also produced antisera that cross-neutralized EBOV, BDBV, and SUDV, when tested in suitable assays.

Based on the above-discussed insights and discoveries, the inventors conceived, and the present disclosure describes, various ebolavirus GP-ferritin fusion proteins, nanoparticles composed of such fusion proteins, nucleic acids, nucleic acid constructs and vectors encoding ebolavirus GP-ferritin fusion proteins, as well as cells, compositions, kits, and methods related to production and use of ebolavirus GP fusion protein. Expression and purification of ebolavirus GP-ferritin fusion proteins can be carried out and scaled using standard protocols for soluble proteins, with the purified fusion proteins self-assembling into homogenous populations of nanoparticles. Ebolavirus GP-ferritin fusion proteins and the related nucleic acids, nucleic acid constructs, vectors, cells, compositions, kits and methods conceived by the inventors and described in the present disclosure are useful for a variety of applications, including, but not limited to, development and production of immunogenic compositions (vaccines), based on proteins or nucleic acids and useful for inducing an immune response against ebolavirus infections, as well as for prevention or treatment of ebolavirus infections, including, but not limited to, EBOV (Ebola or Zaire), BDBV (Bundibugyo), RESTV (Reston), SUDV (Sudan), TAFV (Taï Forest), or Bombali (BOMV) ebolavirus infections.

### Terms and Concepts

A number of terms and concepts are discussed below. They are intended to facilitate the understanding of various embodiments of the invention in conjunction with the rest of the present document and the accompanying figures. These terms and concepts may be further clarified and understood based on the accepted conventions in the fields of the present invention, as well as the description provided throughout the present document and/or the accompanying figures. Some other terms can be explicitly or implicitly defined in other sections of this document and in the accompanying figures, and may be used and understood based on the accepted conventions in the fields of the present invention, the description provided throughout the present document and/or the accompanying figures. The terms not explicitly defined can also be defined and understood based on the accepted conventions in the fields of the present invention and interpreted in the context of the present document and/or the accompanying figures.

Unless otherwise dictated by context, singular terms shall include pluralities, and plural terms shall include the singular. Generally, nomenclatures used in connection with, and techniques of, cell and tissue culture, molecular biology, immunology, microbiology, genetics and protein and nucleic acid chemistry are those well-known and commonly used. Known methods and techniques are generally performed according to conventional methods well-known and as described in various general and more specific references, unless otherwise indicated. The nomenclatures used in connection with the laboratory procedures and techniques described in the present disclosure are those well-known and commonly used.

As used herein, the terms “a”, “an”, and “the” can refer to one or more unless specifically noted otherwise.

The use of the term “or” is used to mean “and/or,” unless explicitly indicated to refer to alternatives only, or the alternatives are mutually exclusive, although the disclosure supports a definition that refers to only alternatives and “and/or.” As used herein “another” can mean at least a second or more.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95×, 0.96×, 0.97×, 0.98×, 0.99×, 1.01×, 1.02×, 1.03×, 1.04×, and 1.05×. Thus, expressions “about X” or “approximately X” are intended to teach and provide written support for a claim limitation of, for example, “0.98×.” Alternatively, in biological systems, the terms “about” and “approximately” may mean values that are within an order of magnitude, preferably within 5-fold, and more preferably within 2-fold of a given value. Numerical quantities given herein are approximate unless stated otherwise, meaning that the term “about” or “approximately” can be inferred when not expressly stated. When “about” is applied to the beginning of a numerical range, it applies to both ends of the range.

The terms “protein,” “peptide,” and “polypeptide” are used interchangeably to refer to a polymer of amino acid residues. The terms apply to naturally occurring amino acid polymers and non-natural amino acid polymers, as well as to amino acid polymers in which one (or more) amino acid residue is an artificial chemical mimetic of a corresponding naturally occurring amino acid. The terms encompass amino acid chains of any length, including full-length proteins, wherein the amino acid residues are linked by covalent peptide bonds.

An “isolated” or “purified” polypeptide or protein, or biologically active portion a polypeptide or a protein, is substantially or essentially free from components that normally accompany or interact with the polypeptide or protein as found in its naturally occurring environment. Thus, an isolated or purified polypeptide or protein is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, 1%, 0.5%, or 0.1% (total protein) of contaminating protein. When the protein of the invention or its biologically active portion is recombinantly produced, optimally culture medium represents less than about 30%, 20%, 10%, 5%, 1%, 0.5%, or 0.1% (by concentration) of chemical precursors or non-protein-of-interest chemicals.

The term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid).

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and their combinations. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and their combinations.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to by either the commonly known three letter symbols or by the one-letter symbols recommended by the IUPAC-IUB Biochemical Nomenclature Commission.

The expression “conservatively modified variant” and related expression may apply to amino acid sequences, as well to nucleic acid sequences encoding amino acid sequences. Substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

The terms “nucleic acid,” “nucleic acid sequence,” “nucleotide sequence,” “oligonucleotide,” “polynucleotide” and the related terms and expressions refer to deoxyribonucleic acids (DNA) or ribonucleic acids (RNA) and their polymers. Nucleic acid sequences, as discussed in the present disclosure, encompass all forms of nucleic acids, including, but not limited to, single-stranded forms, double-stranded forms, hairpins, stem-and-loop structures, and the like. When an RNA sequence is described, its corresponding DNA sequence is also described, wherein uridine is represented as thymidine. When a DNA sequence is described, its corresponding RNA sequence is also described, wherein thymidine is represented as uridine. Unless specifically limited, the term “nucleic acid” and the related terms and expressions encompass nucleic acids containing known analogues of natural nucleotides that have similar properties as the reference nucleic acid, and are metabolized in a manner similar to naturally occurring nucleotides. A nucleic acid sequence can include combinations of deoxyribonucleic acids and ribonucleic acids. Such deoxyribonucleic acids and ribonucleic acids include both naturally occurring molecules and synthetic analogues. Unless otherwise indicated, a particular nucleic acid sequence also implicitly encompasses degenerate codon substitutions, alleles, orthologs, SNPs, and complementary sequences as well as the sequence explicitly indicated. Degenerate codon substitutions may be achieved by generating sequences in which the third position of one or more selected (or all) codons is substituted with mixed-base and/or deoxyinosine residues.

The terms “identity,” “substantial identity,” “similarity,” “substantial similarity,” “homology” and the related terms and expressions used in the context of describing nucleic acid or amino acid sequences refer to a sequence that has at least 60% sequence identity to a reference sequence. Examples include at least: 60%, 65%, 70%, 75%, 80%, 85%, 86%, 87%, 88%, 89%, 90%, 91%, 92%, 93%, 94%, 95%, 96%, 97%, 98%, or 99%, sequence identity, as compared to a reference sequence using the programs for comparison of nucleic acid or amino acid sequences, such as BLAST using standard parameters. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default (standard) program parameters can be used, or alternative parameters can be designated. The sequence comparison algorithm then calculates the percent sequence identities for the test sequences relative to the reference sequence, based on the program parameters. A “comparison window” includes reference to a segment of any one of the number of contiguous positions (from 20 to 600, usually about 50 to about 200, more commonly about 100 to about 150), in which a sequence may be compared to a reference sequence of the same number of contiguous positions after the two sequences are optimally aligned. Methods of alignment of sequences for comparison are well-known. Optimal alignment of sequences for comparison may be conducted, for example, by the local homology algorithm of Smith and Waterman, 1981, by the homology alignment algorithm of Needleman and Wunsch, 1970, by the search for similarity method of Pearson and Lipman, 1988, by computerized implementations of these algorithms (for example, BLAST), or by manual alignment and visual inspection.

Algorithms that are suitable for determining percent sequence identity and sequence similarity include BLAST and BLAST 2.0 algorithms, which are described in Altschul et al., 1990, and Altschul et al., 1977, respectively. Software for performing BLAST analyses is publicly available through the National Center for Biotechnology Information (NCBI) website. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (Henikoff and Henikoff, 1989). The BLAST algorithm also performs a statistical analysis of the similarity between two sequences (Karlin and Altschul, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10⁻²⁰.

The term “antibody” and the related terms refer to an immunoglobulin or its fragment that binds to a particular spatial and polar organization of another molecule. Immunoglobulins include various classes and isotypes, such as IgA, IgD, IgE, IgG1, IgG2a, IgG2b and IgG3, IgG4, IgM, etc. An antibody can be monoclonal or recombinant, and can be prepared by laboratory techniques, such as by preparing continuous hybrid cell lines and collecting the secreted protein, or by cloning and expressing nucleotide sequences or their mutagenized versions coding at least for the amino acid sequences required for binding. The term “antibody” encompasses natural, artificially modified, and artificially generated antibody forms, such as humanized, human, single-chain, chimeric, synthetic, recombinant, hybrid, mutated, grafted, and in vitro generated antibodies and their fragments. The term “antibody” also includes composite forms including but not limited to fusion proteins containing an immunoglobulin moiety. “Antibody” also refers to non-quaternary antibody structures (such as camelids and camelid derivatives). Antibody fragments may include Fab, Fv and F(ab′) 2, Fab′, scFv, Fd, dAb, Fc, and the like. Antibodies may also be single-chain antibodies, chimeric antibodies, humanized antibodies, or any other antibody derivative that retains binding activity that is specific for a particular binding site. In addition, aggregates, polymers and conjugates of immunoglobulins or their fragments can be used where appropriate.

The expression “neutralizing antibody” can refer to an antibody capable of keeping an infectious agent, such as a virus, from infecting a cell by neutralizing or inhibiting one or more parts of the life cycle of the infectious agent. In the context of the present disclosure, neutralizing antibodies can prevent an ebolavirus, such as, but not limited to, Bundibugyo ebolavirus (BDBV), Reston ebolavirus (RESTV), Sudan ebolavirus (SUDV), Taï Forest ebolavirus (TAFV, originally Côthe d'Ivoire ebolavirus), Zaire ebolavirus (Zaire, Ebola or EBOV), or Bombali ebolavirus (BOMV), or any combination thereof, from completing its life cycle in host cell. The life cycle of the virus, for example, an ebolavirus, starts with attachment of the virus to a host cell and ends with budding of a newly formed virus from the host cell. This life cycle includes, but is not limited to, the steps of attaching to a cell, entering a cell, fusion of the viral membrane with the host cell membrane, release of viral ribonucleoproteins into the cytoplasm, formation of new viral particles and budding of viral particles from the host cell membrane.

The term “immunogenic” and the related terms, when used in the context of the present disclosure, refer to the ability of an antigen, which can be a protein, a polypeptide, or a region of a protein or a polypeptide, to elicit in a subject an immune response to the specific antigen. In the context of the present disclosure, an immune response is the development in a subject of a humoral and/or a cellular immune response to an antigen. A “humoral immune response” refers to an immune response mediated by antibody molecules, including secretory (IgA) or IgG molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A cellular immune response also refers to the production of cytokines, chemokines and other such molecules produced by activated T-cells and/or other white blood cells, including those derived from CD4⁺ and CD8⁺ T-cells. Thus, an immunogenic composition can stimulate CTLs, and/or the production or activation of helper T-cells. The production of chemokines and/or cytokines may also be stimulated. An immunogenic composition may also elicit an antibody-mediated immune response. An immunogenic composition may include one or more of the following effects upon administration to a subject: production of antibodies by B-cells; and/or the activation of suppressor, cytotoxic, or helper T-cells and/or T-cells directed specifically to an antigen protein present in the immunogenic composition. Immune response elicited in the subject may serve to neutralize infectivity of a virus, such as an ebolavirus, including, but not limited to, Bundibugyo ebolavirus (BDBV), Reston ebolavirus (RESTV), Sudan ebolavirus (SUDV), Taï Forest ebolavirus (TAFV, originally Côthe d'Ivoire ebolavirus), Zaire ebolavirus (Zaire, Ebola or EBOV), or Bombali ebolavirus (BOMV), or any combination thereof, and/or mediate antibody-complement, or antibody dependent cell cytotoxicity (ADCC) to provide protection against viral infection to an immunized subject. Various aspects of an immune response elicited by an immunogenic composition can be determined using standard assays, some of which are described in the present disclosure.

Immunogenic compositions, as described in the present disclosure, may also be referred to as “vaccines.” Immunogenic compositions, or vaccines, may contain antigens that elicit immune responses to them in a subject upon administration. For example, some immunogenic compositions, or vaccines, described in the present disclosure contain Ebolavirus GP antigens, such as Ebola GP antigens, that can elicit immune responses to them in a subject upon administration. Immunogenic compositions may also contain nucleic acid sequences encoding such antigens. For example, some immunogenic compositions, or vaccines, described in the present disclosure contain nucleic acid sequences encoding Ebolavirus GP antigens, such as Ebola GP antigens. Immunogenic compositions containing antigen-encoding nucleic acid sequences may be described or referred to as “nucleic acid vaccines.” An expression “nucleic acid vaccine” and the related term and expressions encompasses naked DNA vaccines, e.g., plasmid vaccine, and viral vector-based nucleic acids vaccines that are comprised of a viral vector and/or delivered as viral particles.

The term “antigen” refers to a molecule, such as a polypeptide, containing one or more epitopes (either linear, conformational or both) that can stimulate a subject's immune system to produce antigen-specific immune response. A polypeptide epitope may include between about 7 and 15 amino acids, such as, 9, 10, 12 or 15 amino acids. For example, the expression “Ebolavirus GP antigen” may refer to a polypeptide of Ebolavirus GP, such as Ebola GP. The term “antigen” may be used interchangeably with the term “immunogen.”

“Virus” is used in both the plural and singular senses. “Virion” refers to a single virus. For example, the expression “ebolavirus virion” refers to an ebolavirus particle.

Ebolavirus is a genus of viruses in the family Filoviridae, order Mononegavirales. Members of the genus Ebolavirus are referred to as ebolaviruses. The ebolaviruses are enveloped and contain an 18 kb genome of non-segmented, negative sense, single-stranded RNA that encodes seven genes: NP, VP35, VP40, GP, VP30, VP24 and L2. The six known ebolavirus species are named for the region where each was originally identified: Bundibugyo ebolavirus (BDBV), Reston ebolavirus (RESTV), Sudan ebolavirus (SUDV), Taï Forest ebolavirus (TAFV, originally Côthe d'Ivoire ebolavirus), Zaire ebolavirus (Zaire, Ebola or EBOV), and Bombali ebolavirus (BOMV). All but BOMV are known to cause Ebola virus disease in humans, severe hemorrhagic fever with a high fatality rate. Ebolavirus viruses are filamentous, enveloped viruses with non-segmented, single-stranded negative-sense RNA (-ssRNA) genomes that are surrounded by a helical nucleocapsid.

Ebolavirus glycoprotein (GP) is the sole viral surface protein of ebolaviruses. GP gene of ebolaviruses encodes full-length envelope glycoprotein (GP) and its two truncated secreted versions (sGP and ssGP) generated by transcriptional editing. The full-length envelope GP is a trimeric class I viral fusion protein responsible for host cell attachment and entry. The structure of GP is discussed, for example, in Lee et al. 2008 and Peng et al. 2022. Full-length GP is translated as a ~670 amino acid precursor and is cleaved by host furin into two disulfide-linked subunits: GP1 and GP2. GP1 is responsible for receptor binding and consists of four domains: base, head, glycan cap (amino acids 214 to 267 in reference to SEQ ID NO:1) and mucin-like domain (MLD) (amino acids 312 to 462 in reference to SEQ ID NO:1). The GP2 subunit contains the fusion peptide. Following viral entry via endocytosis, GP is proteolytically processed in the endosome (Chandran et al. 2005; Schornberg et al. 2006), where the mucin-like domain (MLD) and the glycan cap (the latter being a poorly conserved region) are cleaved to allow its binding to the receptor Niemann-Pick C1 (Côté et al., 2011; Wang et al. 2016). Subsequently, GP undergoes structural rearrangement to prompt fusion of viral and cellular membranes and transfer of the viral genome into the cytosol (Brecher et al. 2012). GP is the primary target of antibodies produced upon infection or vaccination. Several GP-targeting monoclonal antibodies (mAbs) have been shown to prevent Ebola infection in nonhuman primates (NHPs) (Corti et al. 2016; Pascal et al. 2014; Qiu et al. 2014) and humans (Mulangu et al. 2019; PREVAIL II Writing Group et al. 2016). One example of a monoclonal antibody is mAb114 (Ebanga), that binds to the core receptor binding domain of the Zaire ebolavirus GP and prevents the virus from infecting human cells. mAb114 can also protect monkeys from lethal Ebola virus disease when given as late as five days after infection. Two antibody drugs that bind GP and block viral entry (Ebanga and Inmazeb) are approved to treat Ebola virus disease (FDA, 2020). Neutralizing activity of antibodies is an immune correlate of protection in NHPs and humans against infection (Maruyama et al. 1999; Rijal et al. 2019; Marzi et al. 2013). Several GP-targeting mAbs have been isolated that neutralize EBOV, SUDV and BDBV (Flyak et al. 2016; Flyak et al. 2018; Wec et al. 2017; Zhao et al. 2017; Luczkowiak et al. 2018). The amino acid sequence of the GP protein of these viruses is shown in Table 1 below.

TABLE 1
Ebolavirus Glycoprotein (GP) Protein Sequences

“Wild-type” amino acid sequence of Zaire (Ebola) ebolavirus GP protein -
SEQ ID NO: 1
MGVTGILQLPRDRFKRTSFFLWVIILFQRTFSIPLGVIHNSTLQVSDVDKLVCRDKLS
STNQLRSVGLNLEGNGVATDVPSATKRWGFRSGVPPKVVNYEAGEWAENCYNLEI
KKPDGSECLPAAPDGIRGFPRCRYVHKVSGTGPCAGDFAFHKEGAFFLYDRLASTVI
YRGTTFAEGVVAFLILPQAKKDFFSSHPLREPVNATEDPSSGYYSTTIRYQATGFGT
NETEYLFEVDNLTYVQLESRFTPQFLLQLNETIYTSGKRSNTTGKLIWKVNPEIDTTI
GEWAFWETKKNLTRKIRSEELSFTVVSNGAKNISGQSPARTSSDPGTNTTTEDHKIM
ASENSSAMVQVHSQGREAAVSHLTTLATISTSPQSLTTKPGPDNSTHNTPVYKLDIS
EATQVEQHHRRTDNDSTASDTPSATTAAGPPKAENTNTSKSTDFLDPATTTSPQNH
SETAGNNNTHHQDTGEESASSGKLGLITNTIAGVAGLITGGRRTRREAIVNAQPKCN
PNLHYWTTQDEGAAIGLAWIPYFGPAAEGIYIEGLMHNQDGLICGLRQLANETTQA
LQLFLRATTELRTFSILNRKAIDFLLQRWGGTCHILGPDCCIEPHDWTKNITDKIDQII
HDFVDKTLPDQGDNDNWWTGWRQWIPAGIGVTGVIIAVIALFCICKFVF
“Wild-type” amino acid sequence of Bundibugyo ebolavirus GP protein -
SEQ ID NO: 2
MVTSGILQLPRERFRKTSFFVWVIILFHKVFPIPLGVVHNNTLQVSDIDKLVCRDKLS
STSQLKSVGLNLEGNGVATDVPTATKRWGFRAGVPPKVVNYEAGEWAENCYNLD
IKKADGSECLPEAPEGVRGFPRCRYVHKVSGTGPCPEGYAFHKEGAFFLYDRLASTI
IYRSTTFSEGVVAFLILPETKKDFFQSPPLHEPANMTTDPSSYYHTVTLNYVADNFGT
NMTNFLFQVDHLTYVQLEPRFTPQFLVQLNETIYTNGRRSNTTGTLIWKVNPTVDT
GVGEWAFWENKKNFTKTLSSEELSVIFVPRAQDPGSNQKTKVTPTSFANNQTSKNH
EDLVPEDPASVVQVRDLQRENTVPTPPPDTVPTTLIPDTMEEQTTSHYEPPNISRNHQ
ERNNTAHPETLANNPPDNTTPSTPPQDGERTSSHTTPSPRPVPTSTIHPTTRETHIPTT
MTTSHDTDSNRPNPIDISESTEPGPLTNTTRGAANLLTGSRRTRREITLRTQAKCNPN
LHYWTTQDEGAAIGLAWIPYFGPAAEGIYTEGIMHNQNGLICGLRQLANETTQALQ
LFLRATTELRTFSILNRKAIDFLLQRWGGTCHILGPDCCIEPHDWTKNITDKIDQIIHD
FIDKPLPDQTDNDNWWTGWRQWVPAGIGITGVIIAVIALLCICKFLL
“Wild-type” amino acid sequence of Reston ebolavirus GP protein - SEQ ID NO: 3
MGSGYQLLQLPRERFRKTSFLVWVIILFQRAISMPLGIVINSTLKATEIDQLVCRDKL
SSTSQLKSVGLNLEGNGIATDVPSATKRWGFRSGVPPKVVSYEAGEWAENCYNLEI
KKSDGSECLPLPPDGVRGFPRCRYVHKVQGTGPCPGDLAFHKNGAFFLYDRLASTV
IYRGTTFTEGVVAFLILSEPKKHFWKATPAHEPVNTTDDSTSYYMTLTLSYEMSNFG
GKESNTLFKVDNHTYVQLDRPHTPQFLVQLNETLRRNNRLSNSTGRLTWTLDPKIE
PDVGEWAFWETKKNFSQQLHGENLHFQILSTHTNNSSDQSPAGTVQGKISYHPPTN
NSELVPTDSPPVVSVLTAGRTEEMSTQGLTNGETITGFTANPMTTTIAPSPTMTSEVD
NNVPSEQPNNTASIEDSPPSASNETIDHSEMNPIQGSNNSAQSPQTKTTPAPTASPMT
QDPQETANSSKLGTSPGSAAEPSQPGFTINTVSKVADSLSPTRKQKRSVRQNTANKC
NPDLHYWTAVDEGAAVGLAWIPYFGPAAEGIYIEGVMHNQNGLICGLRQLANETT
QALQLFLRATTELRTYSLLNRKAIDFLLQRWGGTCRILGPSCCIEPHDWTKNITDEIN
QIKHDFIDNPLPDHGDDLNLWTGWRQWIPAGIGIIGVIIAIIALLCICKILC
“Wild-type” amino acid sequence of Sudan ebolavirus GP protein - SEQ ID NO: 4
MGGLSLLQLPRDKFRKSSFFVWVIILFQKAFSMPLGVVTNSTLEVTEIDQLVCKDHL
ASTDQLKSVGLNLEGSGVSTDIPSATKRWGFRSGVPPKVVSYEAGEWAENCYNLEI
KKPDGSECLPPPPDGVRGFPRCRYVHKAQGTGPCPGDYAFHKDGAFFLYDRLAST
VIYRGVNFAEGVIAFLILAKPKETFLQSPPIREAVNYTENTSSYYATSYLEYEIENFGA
QHSTTLFKIDNNTFVRLDRPHTPQFLFQLNDTIHLHQQLSNTTGRLIWTLDANINADI
GEWAFWENKKNLSEQLRGEELSFEALSLNETEDDDAASSRITKGRISDRATRKYSD
LVPKNSPGMVPLHIPEGETTLPSQNSTEGRRVGVNTQETITETAATIIGTNGNHMQIS
TIGIRPSSSQIPSSSPTTAPSPEAQTPTTHTSGPSVMATEEPTTPPGSSPGPTTEAPTLTT
PENITTAVKTVLPQESTSNGLITSTVTGILGSLGLRKRSRRQTNTKATGKCNPNLHY
WTAQEQHNAAGIAWIPYFGPGAEGIYTEGLMHNQNALVCGLRQLANETTQALQLF
LRATTELRTYTILNRKAIDFLLRRWGGTCRILGPDCCIEPHDWTKNITDKINQIIHDFI
DNPLPNQDNDDNWWTGWRQWIPAGIGITGIIIAIIALLCVCKLLC
“Wild-type” amino acid sequence of Tai Forest ebolavirus GP protein - SEQ ID NO: 5
MGASGILQLPRERFRKTSFFVWVIILFHKVFSIPLGVVHNNTLQVSDIDKFVCRDKLS
STSQLKSVGLNLEGNGVATDVPTATKRWGFRAGVPPKVVNYEAGEWAENCYNLA
IKKVDGSECLPEAPEGVRDFPRCRYVHKVSGTGPCPGGLAFHKEGAFFLYDRLASTI
IYRGTTFAEGVIAFLILPKARKDFFQSPPLHEPANMTTDPSSYYHTTTINYVVDNFGT
NTTEFLFQVDHLTYVQLEARFTPQFLVLLNETIYSDNRRSNTTGKLIWKINPTVDTS
MGEWAFWENKKNFTKTLSSEELSFVPVPETQNQVLDTTATVSPPISAHNHAGEDHK
ELVSEDSTPVVQMQNIKGKDTMPTTVTGVPTTTPSPFPINARNTDHTKSFIGLEGPQE
DHSTTQPAKTTSQPTNSTESTTLNPTSEPSSRGTGPSSPTVPNTTESHAELGKTTPTTL
PEQHTAASAIPRAVHPDELSGPGFLTNTIRGVTNLLTGSRRKRRDVTPNTQPKCNPN
LHYWTALDEGAAIGLAWIPYFGPAAEGIYTEGIMENQNGLICGLRQLANETTQALQ
LFLRATTELRTFSILNRKAIDFLLQRWGGTCHILGPDCCIEPQDWTKNITDKIDQIIHD
FVDNNLPNQNDGSNWWTGWKQWVPAGIGITGVIIAIIALLCICKFML
“Wild-type” amino acid sequence of Bombali ebolavirus GP protein - SEQ ID NO: 6
MILQVPEKRHQRTVLFIWLVILFQRAVSVPLGVIHNSTLQVSDIDKFVCHDKLTSTN
QLRSIGLNLEGNGIATDVPSATKRWGFRAGVPPKVVGYEAGEWAENCYNLEIKKP
DGSECLPMAPEGIRGFPRCRYVHKVSGTGSCESGFAFHKEGAFFLYDRLASTIIYRG
TTFAEGVVAFIILPKAEKNFLQPPLTQGPTNTTNDPSSMYHSTTLEYETTRFGTNRSA
FWFKVDNLTFVQLESRFTPQFLVELNETIYIEGKRSNTTGRLIWQVNSRVDTDVGE
WAFWENKKNLKKSFPREELSLTAVPRAADSEHDAHPPEYTPGPDSNPTINDNTELV
TEDPAHLVQLQRQGRKEILPTTIPQAIEREPPAAQHDNPRNSPTPPSPIESDITDSTQA
EDLTHTDDPSTINSATEEPLPEVGETTQVPRDPEGPRRTQPTPPTGQPEQPSDNTMTP
GQIHSESAAPMGERSIDGPGLLTNTLAGVARLITGAGRAKRESPEIRGAKCNPNLHY
WTTHEESAAAGLAWIPYFGPAAEGIYTEGLMQNQNELICGLRQLANETTQALQLFL
RSTTELRTFSILNRKAIDFLLQRWGGTCRILGPDCCIEPHDWTKNITDRIDQIIHDFVD
KPLPDQSNNDNWWTGWRQWIPAGIGVVGVIAAFIALICICKIIC

A “domain” of a protein or a polypeptide refers to a region of the protein or polypeptide defined by structural and/or functional properties. Exemplary function properties include enzymatic activity and/or the ability to bind to or be bound by another protein or non-protein entity. For example, GP1 subunit of Ebolavirus GP protein contains base, head, glycan cap and mucin-like domains.

The term “oligomer” and related terms, when used in reference to polypeptides or proteins, refer to complexes formed by two or more polypeptide or protein monomers, which can also be referred to as “subunits” or “chains.” For example, a trimer is an oligomer formed by three polypeptide subunits.

The terms “fusion protein,” “fusion polypeptide,” and the related terms relate to polypeptide molecules, including artificial or engineered polypeptide molecules, that include two or more amino acid sequences previously found in separate polypeptide molecules, that are joined or linked in a fusion protein amino acid sequence to form a single polypeptide. For example, a fusion protein can be an engineered recombinant protein containing amino acid sequence from at least two unrelated proteins that have been joined together, via a peptide bond, to make a single protein. In this context, proteins are considered unrelated, if their amino acid sequences are not normally found joined together via a peptide bond in their natural environment, for example, inside a cell. For example, the present disclosure describes fusion proteins that include an amino acid sequence of an ebolavirus GP protein and an amino acid sequence of a ferritin subunit polypeptide, which are unrelated proteins. The amino acid sequences of a fusion protein are encoded by corresponding nucleic acid sequences that are joined “in frame,” so that they are transcribed and translated to produce a single polypeptide. The amino acid sequences of a fusion protein can be contiguous or separated by one or more spacer, linker or hinge sequences. Fusion proteins can include additional amino acid sequences, such as, for example, signal sequences, tag sequences, and/or linker sequences.

Ferritin is a globular protein found in animals, bacteria, and plants, that acts primarily to control the rate and location of polynuclear Fe (III) 203 formation through the transportation of hydrated iron ions and protons to and from a mineralized core. The globular form of ferritin is made up of monomeric subunit proteins (also referred to as monomeric ferritin subunits), which are polypeptides having a molecule weight of approximately 17-20 kDa. An example of the sequence of one such monomeric ferritin subunit is represented by SEQ ID NO:7 as set forth in Table 2 below. Each monomeric ferritin subunit has the topology of a helix bundle which includes a four antiparallel helix motif, with a fifth shorter helix (the c-terminal helix) lying roughly perpendicular to the long axis of the four-helix bundle. According to convention, the helices are labeled ‘A, B, C, and D & E’ from the N-terminus respectively. The N-terminal sequence lies adjacent to the capsid three-fold axis and extends to the surface, while the E helices pack together at the four-fold axis with the C-terminus extending into the particle core. The consequence of this packing creates two pores on the capsid surface. It is expected that one or both of these pores represent the point by which the hydrated iron diffuses into and out of the capsid. Following production, these monomeric ferritin subunit proteins self-assemble into the globular ferritin protein. Thus, the globular form of ferritin comprises 24 monomeric, ferritin subunit proteins, and has a capsid-like structure having 432 symmetry.

TABLE 2
Helicobacter pylori Ferritin Subunit Sequence

Amino acid sequence of Helicobacter pylori
ferritin subunit with the N-terminal deletion of
the first five amino acids - SEQ ID NO: 7
DIIKLLNEQVNKEMQSSNLYMSMSSWCYTHSLDGAGLFLFDHAAEEYEHA
KKLIIFLNENNVPVQLTSISAPEHKFEGLTQIFQKAYEHEQHISESINNI
VDHAIKSKDHATFNFLQWYVAEQHEEEVLFKDILDKIELIGNENHGLYLA
DQYVKGIAKSRKS

The terms “individual”, “subject”, and “patient” can be used interchangeably in the present disclosure to refer to a non-human animal or a human. Examples of subjects include, but are not limited to: humans and other primates, including non-human primates, such as chimpanzees and other apes and monkey species; farm animals, such as cattle, sheep, pigs, goats and horses; domestic mammals such as dogs and cats; laboratory animals including rodents, such as mice, rats and guinea pigs; birds, including domestic, wild and game birds, such as chickens, turkeys and other gallinaceous birds, ducks, geese, and the like. The terms individual, subject, and patient, by themselves, do not denote a particular age, sex, race, or clinical status. Thus, subjects of any age, whether male or female, are intended to be covered by the present disclosure and include, but are not limited to the elderly, adults, children, babies, infants, and toddlers. Likewise, the methods of the present invention can be applied to any human race, including, for example, Caucasian (white), African-American (black), Native American, Native Hawaiian, Hispanic, Latino, Asian, and European. An infected subject is a subject that is known to have been infected by an infection organism, such as ebolavirus.

The terms “administering” or “administration,” when using in the context of administration of a composition described in the present disclosure to a subject (and the related terms and expression), refer to the act of physically delivering a substance as it exists outside the body (for example, an immunogenic composition described in the present disclosure) into a subject. Administration can be by mucosal, intradermal, intravenous, intramuscular, subcutaneous delivery and/or by any other known methods of physical delivery. Administration encompasses direct administration, such as administration to a subject by a medical professional or self-administration, or indirect administration, which may be the act of prescribing a composition described in the present disclosure.

The term “glycosylation” and the related terms and expressions refer to a process and/or result of post-translational modification of proteins and polypeptides that adds carbohydrate moieties (also referred to as “glycans”) to certain amino acids of a polypeptide or protein molecules. In N-linked glycosylation, a carbohydrate moiety is added to asparagine. In O-linked glycosylation, a carbohydrate moiety is added to serine or threonine. Attachment of the carbohydrate moiety requires recognition of a consensus amino acid sequence (“consensus sequence”).

### Fusion Proteins and Nanoparticles

Provided in this disclosure and included among the embodiments of the present invention are fusion proteins comprising an artificially modified amino acid sequence of a GP protein of an ebolavirus (“ebolavirus GP protein”) and an amino acid sequence of a ferritin subunit polypeptide. An artificially modified ebolavirus GP protein amino acid sequence included in the fusion proteins according to the embodiments of the present invention may also be referred to as “GP polypeptide,” “GP protein domain” or “GP domain,” while the ferritin subunit polypeptide amino acid sequence may be referred to as “ferritin amino acid sequence,” “ferritin”, “ferritin domain”, or “ferritin polypeptide.” In addition to the above amino acid sequences, fusion proteins according to the embodiments of the present invention can include other amino acid sequences such as, but not limited to, amino acid sequence of polypeptide domains other than GP domain and ferritin domains, linker sequences, signal sequences, tags, etc. Some of these other amino acid sequences are described elsewhere in the present disclosure.

An artificially modified amino acid sequence of an ebolavirus GP protein included in a fusion protein according to embodiments of the present invention can be derived from a GP protein sequence from any ebolavirus species, such as, but not limited to, Bundibugyo ebolavirus (BDBV), Reston ebolavirus (RESTV), Sudan ebolavirus (SUDV), Taï Forest ebolavirus (TAFV, originally Côthe d'Ivoire ebolavirus), Zaire ebolavirus (Zaire, Ebola or EBOV), or Bombali ebolavirus (BOMV). It is to be understood that an ebolavirus GP protein sequence may be a full or a partial amino acid sequence of an ebolavirus GP protein, an amino acid sequence of a fragment of an ebolavirus GP protein, or an amino acid sequence of a variant of an ebolavirus GP protein, including naturally occurring and artificially generated variants, including artificial GP glycosylation variants according to the embodiments of the present invention. Some embodiments of the fusion proteins may contain an artificially modified naturally occurring (or “wild-type”) amino acid sequence of an ebolavirus GP protein or a portion thereof. Some embodiments of the fusion proteins may contain an artificially modified amino acid sequence of an ebolavirus GP protein derived from naturally occurring (or “wild-type”) amino acid sequence of an ebolavirus GP protein (such as SEQ ID NO:1, SEQ ID NO:2, SEQ ID NO:3, SEQ ID NO:4, SEQ ID NO:5, or SEQ ID NO:6) or a portion or portions thereof. Exemplary embodiments of such artificially modified amino acid sequence of an ebolavirus GP protein are SEQ ID NO:12, SEQ ID NO:13, SEQ ID NO:14, and SEQ ID NO:15. Exemplary embodiments of fusion protein sequences incorporating artificially modified amino acid sequence of an ebolavirus GP protein are SEQ ID NO:8, SEQ ID NO:9, SEQ ID NO: 10, and SEQ ID NO: 11. In some non-limiting examples, artificially modified amino acid sequences may contain one or more features of the wild-type amino acid sequences of an ebolavirus GP protein sequences, such as, but not limited to, those discussed in the present disclosure. In some exemplary embodiments, the features of the wild-type amino acid sequences of an ebolavirus GP protein sequences may be combined in ways that are not found naturally occurring sequence. For example, an artificially modified amino acid sequence of an ebolavirus GP protein or a portion thereof may include one or more features from each of two or more naturally circulating ebolaviruses, such as, but not limited to, Bundibugyo ebolavirus (BDBV), Reston ebolavirus (RESTV), Sudan ebolavirus (SUDV), Taï Forest ebolavirus (TAFV, originally Côthe d'Ivoire ebolavirus), Zaire ebolavirus (Zaire, Ebola or EBOV), and Bombali ebolavirus (BOMV). In some embodiments, artificially modified ebolavirus GP proteins may contain deletions of a portion or portions of amino acid sequence with respect to wild-type amino acid sequences, such as a deletion of the mucin-like domain or a portion or portions thereof. In some embodiments, artificially modified ebolavirus GP proteins may contain substitutions of a portion or portions of amino acid sequence with respect to wild-type amino acid sequences, such as a substitution of a transmembrane domain (amino acids 651 to 671 in reference to SEQ ID NO:1) or a portion thereof. In some example, a transmembrane domain may be substituted with a GCN4 trimerization domain (MKQIEDKIEEILSKIYHIENEIARIKKLIGEV; SEQ ID NO:21; Misasi et al. 2016) or foldon trimerization domain (GYIPEAPRDGQAYVRKDGEWVLLSTFL; SEQ ID NO:24; Meieret et al. 2004). Artificially modified amino acid sequences of ebolavirus GP proteins may contain various amino acid modifications, as compared to wild-type sequences. For example, an artificially modified amino acid sequence of an ebolavirus GP protein may contain mutations removing or adding glycosylation sites. In some embodiments, an artificially modified amino acid sequence of the ebolavirus GP comprises at least two newly added artificial glycosylation sites. In some embodiments, an artificially modified amino acid sequence of the ebolavirus GP comprises two, three, four, five, six, seven, eight, nine, ten, etc. newly added artificial glycosylation sites. In some embodiments, the newly added artificial glycosylation sites are located in glycan cap domain of the ebolavirus GP. For example, an artificially modified amino acid sequence of the ebolavirus GP may contain two, three, or four newly added artificial glycosylation sites in a glycan cap domain. In some embodiments, an artificially modified amino acid sequence of the ebolavirus GP contains two or more amino acid substitutions (e.g., two, three, or four substitutions) at positions corresponding to positions 212, 214, 251, 280, 272, 274, 212, and 214 of SEQ ID NO:12 (such as, for example, Q212N, E214T, D251N, L280N, S272N, and E274T), with the amino acid substitution resulting in introduction of artificial glycosylation sites. Non-limiting examples of such amino acid sequences are artificially modified amino acid sequences of an ebolavirus GP protein that are incorporated into fusion proteins are sequences with at least 90% sequence identity (e.g., at least 90%, at least 91%, at least 92%, at least 93%, at least 94%, at least 95%, at least 96%, at least 97%, at least 98%, or at least 99%, or 100% percent identity) to SEQ ID NO: 13, SEQ ID NO:14, or SEQ ID NO:15. Non-limiting examples of fusion proteins incorporating artificially modified amino acid sequences of an ebolavirus GP protein with newly added artificial glycosylation sites are sequences with at least 90% sequence identity (e.g., at least 90%, at least 91%, at least 92%, at least 93%, at least 94%, at least 95%, at least 96%, at least 97%, at least 98%, or at least 99%, or 100% percent identity) to SEQ ID NO:9, SEQ ID NO:10, or SEQ ID NO:11. These sequences are provided in Table 3 below (SGG artificial substitutions are shown in bold and italics; artificial glycosylation sites are shown in bold; SGG linker sequence is shown in italics; ferritin sequence is shown in Courier font).

TABLE 3
Exemplary Ebolavirus GP Fusion Protein Sequences

Amino acid sequence of GP-Fer fusion protein - SEQ ID NO: 8
MGVTGILQLPRDRFKRTSFFLWVIILFQRTFSIPLGVIHNSTLQVSDVDKLVCRDKL
SSTNQLRSVGLNLEGNGVATDVPSATKRWGFRSGVPPKVVNYEAGEWAENCYNL
EIKKPDGSECLPAAPDGIRGFPRCRYVHKVSGTGPCAGDFAFHKEGAFFLYDRLAS
TVIYRGTTFAEGVVAFLILPQAKKDFFSSHPLREPVNATEDPSSGYYSTTIRYQATG
FGTNETEYLFEVDNLTYVQLESRFTPQFLLQLNETIYTSGKRSNTTGKLIWKVNPEI
DTTIGEWAFWETKKNLTRKIRSEELSFAGLITGGRRTRREAIVNAQPKCNPNLHYW
TTQDEGAAIGLAWIPYFGPAAEGIYIEGLMHNQDGLICGLRQLANETTQALQLFLR
ATTELRTFSILNRKAIDFLLQRWGGTCHILGPDCCIEPHDWTKNITDKIDQIIHDFVD
KTLPDQGDNDNWWTGWRQWIPAGISGGDIIKLLNEQVNKEMQSSNLYMSMSSWCYTH
SLDGAGLFLFDHAAEEYEHAKKLIIFLNENNVPVQLTSISAPEHKFEGLTQIFQKAYEHEQ
HISESINNIVDHAIKSKDHATENFLQWYVAEQHEEEVLEKDILDKIELIGNENHGLYLADQ
YVKGIAKSRKS
Amino acid sequence of GP+2-Fer fusion protein (artificial substitutions D282N and
L311N) - SEQ ID NO: 9
MGVTGILQLPRDRFKRTSFFLWVIILFQRTFSIPLGVIHNSTLQVSDVDKLVCRDKL
SSTNQLRSVGLNLEGNGVATDVPSATKRWGFRSGVPPKVVNYEAGEWAENCYNL
EIKKPDGSECLPAAPDGIRGFPRCRYVHKVSGTGPCAGDFAFHKEGAFFLYDRLAS
TVIYRGTTFAEGVVAFLILPQAKKDFFSSHPLREPVNATEDPSSGYYSTTIRYQATG
FGTNETEYLFEVDNLTYVQLESRFTPQFLLQLNETIYTSGKRSNTTGKLIWKVNPEI
NTTIGEWAFWETKKNLTRKIRSEELSFAGNITGGRRTRREAIVNAQPKCNPNLHY
WTTQDEGAAIGLAWIPYFGPAAEGIYIEGLMHNQDGLICGLRQLANETTQALQLFL
RATTELRTFSILNRKAIDFLLQRWGGTCHILGPDCCIEPHDWTKNITDKIDQIIHDFV
DKTLPDQGDNDNWWTGWRQWIPAGISGGDIIKLLNEQVNKEMQSSNLYMSMSSWCY
THSLDGAGLFLFDHAAEEYEHAKKLIIFLNENNVPVQLTSISAPEHKFEGLTQIFQKAYEH
EQHISESINNIVDHAIKSKDHATFNFLQWYVAEQHEEEVLFKDILDKIELIGNENHGLYLA
DQYVKGIAKSRKS
Amino acid sequence of GP + 3-Fer fusion protein (artificial substitutions D282N,
L311N, S303N, and E305T) - SEQ ID NO: 10
MGVTGILQLPRDRFKRTSFFLWVIILFQRTFSIPLGVIHNSTLQVSDVDKLVCRDKL
SSTNQLRSVGLNLEGNGVATDVPSATKRWGFRSGVPPKVVNYEAGEWAENCYNL
EIKKPDGSECLPAAPDGIRGFPRCRYVHKVSGTGPCAGDFAFHKEGAFFLYDRLAS
TVIYRGTTFAEGVVAFLILPQAKKDFFSSHPLREPVNATEDPSSGYYSTTIRYQATG
FGTNETEYLFEVDNLTYVQLESRFTPQFLLQLNETIYTSGKRSNTTGKLIWKVNPEI
NTTIGEWAFWETKKNLTRKIRNETLSFAGNITGGRRTRREAIVNAQPKCNPNLHY
WTTQDEGAAIGLAWIPYFGPAAEGIYIEGLMHNQDGLICGLRQLANETTQALQLFL
RATTELRTFSILNRKAIDFLLQRWGGTCHILGPDCCIEPHDWTKNITDKIDQIIHDFV
DKTLPDQGDNDNWWTGWRQWIPAGISGGDIIKLLNEQVNKEMQSSNLYMSMSSWCY
THSLDGAGLFLFDHAAEEYEHAKKLIIFLNENNVPVQLTSISAPEHKFEGLTQIFQKAYEH
EQHISESINNIVDHAIKSKDHATFNFLQWYVAEQHEEEVLFKDILDKIELIGNENHGLYLA
DQYVKGIAKSRKS
Amino acid sequence of GP + 4-Fer fusion protein (artificial substitutions D282N,
L311N, S303N, E305T, Q243N, and E245T) - SEQ ID NO: 11
MGVTGILQLPRDRFKRTSFFLWVIILFQRTFSIPLGVIHNSTLQVSDVDKLVCRDKL
SSTNQLRSVGLNLEGNGVATDVPSATKRWGFRSGVPPKVVNYEAGEWAENCYNL
EIKKPDGSECLPAAPDGIRGFPRCRYVHKVSGTGPCAGDFAFHKEGAFFLYDRLAS
TVIYRGTTFAEGVVAFLILPQAKKDFFSSHPLREPVNATEDPSSGYYSTTIRYQATG
FGTNETEYLFEVDNLTYVNLTSRFTPQFLLQLNETIYTSGKRSNTTGKLIWKVNPEI
NTTIGEWAFWETKKNLTRKIRNETLSFAGNITGGRRTRREAIVNAQPKCNPNLHY
WTTQDEGAAIGLAWIPYFGPAAEGIYIEGLMHNQDGLICGLRQLANETTQALQLFL
RATTELRTFSILNRKAIDFLLQRWGGTCHILGPDCCIEPHDWTKNITDKIDQIIHDFV
DKTLPDQGDNDNWWTGWRQWIPAGISGGDIIKLLNEQVNKEMQSSNLYMSMSSWCY
THSLDGAGLFLFDHAAEEYEHAKKLIIFLNENNVPVQLTSISAPEHKFEGLTQIFQKAYEH
EQHISESINNIVDHAIKSKDHATFNFLQWYVAEQHEEEVLFKDILDKIELIGNENHGLYLA
DQYVKGIAKSRKS

In some embodiments, an artificially modified amino acid sequence of ebolavirus GP protein included in a fusion protein as provided herein is an amino acid sequence with at least 80%, at least 85%, at least 90%, at least 91%, at least 92%, at least 93%, at least 94%, at least 95%, at least 96%, at least 97%, at least 98%, or at least 99%, or 100% sequence identity to an artificially modified amino acid sequence of ebolavirus GP protein amino acid sequence, such as, but not limited to, SEQ ID NO: 12, SEQ ID NO:13, SEQ ID NO:14, and SEQ ID NO:15. These sequences are shown in Table 4 below (artificial substitutions are shown in bold and italics; artificial glycosylation sites are shown in bold). In some instances, an amino acid sequence of an ebolavirus GP protein included in a fusion protein as provided herein is a conservatively modified variant of an artificially modified ebolavirus GP protein amino acid sequence comprising one or more amino acid residue substitutions. In some instances, an amino acid sequence of an artificially modified ebolavirus GP protein included in a fusion protein as provided herein comprises a deletion of one or more amino acid residues at the C-terminal, N-terminal, and/or middle portion of the amino acid sequence of an artificially modified ebolavirus GP protein according to the present disclosure. In some instances, the deletion may comprise a one or more consecutive amino acid residues. In some instances, the deletion may comprise a one or more non-consecutive amino acid residues. In some instances, an amino acid sequence of an artificially modified ebolavirus GP protein may comprise a deletion of 1, 2, 3, 4, 5, 6, 7, 8, 9, or 10 amino acid residues of the amino acid sequence of an artificially modified ebolavirus GP protein according to the present disclosure. In some instances, an amino acid sequence of an artificially modified ebolavirus GP protein may comprise a deletion of 10, 15, 20, 25, 30, 35, 40, 45, 50, 55, 60, 65, 70, 75, 80, 85, 90, 95, or 100 amino acid residues, such as deletions of 10-15, 15-30, 25-50, 10-50, or 50-100 amino acid residues of the amino acid sequence of an artificially modified ebolavirus GP protein according to the present disclosure.

TABLE 4
Exemplary Ebolavirus GP Fusion Protein Sequences Included in a Fusion
Protein

Amino acid sequence of ebolavirus GP protein included in a fusion protein -
SEQ ID NO: 12
SIPLGVIHNSTLQVSDVDKLVCRDKLSSTNQLRSVGLNLEGNGVATDVPSATKRW
GFRSGVPPKVVNYEAGEWAENCYNLEIKKPDGSECLPAAPDGIRGFPRCRYVHKV
SGTGPCAGDFAFHKEGAFFLYDRLASTVIYRGTTFAEGVVAFLILPQAKKDFFSSHP
LREPVNATEDPSSGYYSTTIRYQATGFGTNETEYLFEVDNLTYVQLESRFTPQFLLQ
LNETIYTSGKRSNTTGKLIWKVNPEIDTTIGEWAFWETKKNLTRKIRSEELSFAGLIT
GGRRTRREAIVNAQPKCNPNLHYWTTQDEGAAIGLAWIPYFGPAAEGIYIEGLMH
NQDGLICGLRQLANETTQALQLFLRATTELRTFSILNRKAIDFLLQRWGGTCHILGP
DCCIEPHDWTKNITDKIDQIIHDFVDKTLPDQGDNDNWWTGWRQWIPAGI
Amino acid sequence of GP + 2 ebolavirus GP protein included in a fusion protein
(artificial substitutions D251N and L280N) - SEQ ID NO: 13
SIPLGVIHNSTLQVSDVDKLVCRDKLSSTNQLRSVGLNLEGNGVATDVPSATKRW
GFRSGVPPKVVNYEAGEWAENCYNLEIKKPDGSECLPAAPDGIRGFPRCRYVHKV
SGTGPCAGDFAFHKEGAFFLYDRLASTVIYRGTTFAEGVVAFLILPQAKKDFFSSHP
LREPVNATEDPSSGYYSTTIRYQATGFGTNETEYLFEVDNLTYVQLESRFTPQFLLQ
LNETIYTSGKRSNTTGKLIWKVNPEINTTIGEWAFWETKKNLTRKIRSEELSFAGNI
TGGRRTRREAIVNAQPKCNPNLHYWTTQDEGAAIGLAWIPYFGPAAEGIYIEGLM
HNQDGLICGLRQLANETTQALQLFLRATTELRTFSILNRKAIDFLLQRWGGTCHILG
PDCCIEPHDWTKNITDKIDQIIHDFVDKTLPDQGDNDNWWTGWRQWIPAGISGG
Amino acid sequence of GP + 3 ebolavirus GP protein included in a fusion protein
(artificial substitutions D251N, L280N, S272N, and E274T) - SEQ ID NO: 14
SIPLGVIHNSTLQVSDVDKLVCRDKLSSTNQLRSVGLNLEGNGVATDVPSATKRW
GFRSGVPPKVVNYEAGEWAENCYNLEIKKPDGSECLPAAPDGIRGFPRCRYVHKV
SGTGPCAGDFAFHKEGAFFLYDRLASTVIYRGTTFAEGVVAFLILPQAKKDFFSSHP
LREPVNATEDPSSGYYSTTIRYQATGFGTNETEYLFEVDNLTYVQLESRFTPQFLLQ
LNETIYTSGKRSNTTGKLIWKVNPEINTTIGEWAFWETKKNLTRKIRNETLSFAGNI
TGGRRTRREAIVNAQPKCNPNLHYWTTQDEGAAIGLAWIPYFGPAAEGIYIEGLM
HNQDGLICGLRQLANETTQALQLFLRATTELRTFSILNRKAIDFLLQRWGGTCHILG
PDCCIEPHDWTKNITDKIDQIIHDFVDKTLPDQGDNDNWWTGWRQWIPAGI
Amino acid sequence of GP + 4 ebolavirus GP protein included in a fusion protein
(artificial substitutions D251N, L280N, S272N, E274T, Q212N, and E214T) -
SEQ ID NO: 15
QRTFSIPLGVIHNSTLQVSDVDKLVCRDKLSSTNQLRSVGLNLEGNGVATDVPSAT
KRWGFRSGVPPKVVNYEAGEWAENCYNLEIKKPDGSECLPAAPDGIRGFPRCRYV
HKVSGTGPCAGDFAFHKEGAFFLYDRLASTVIYRGTTFAEGVVAFLILPQAKKDFF
SSHPLREPVNATEDPSSGYYSTTIRYQATGFGTNETEYLFEVDNLTYVNLTSRFTPQ
FLLQLNETIYTSGKRSNTTGKLIWKVNPEINTTIGEWAFWETKKNLTRKIRNETLSF
AGNITGGRRTRREAIVNAQPKCNPNLHYWTTQDEGAAIGLAWIPYFGPAAEGIYIE
GLMHNQDGLICGLRQLANETTQALQLFLRATTELRTFSILNRKAIDFLLQRWGGTC
HILGPDCCIEPHDWTKNITDKIDQIIHDFVDKTLPDQGDNDNWWTGWRQWIPAGI

Fusion proteins according to the embodiments of the present invention include an amino acid sequence of a ferritin subunit polypeptide (referred to in this disclosure as a “ferritin amino acid sequence”). The ferritin amino acid sequence can be an amino acid sequence of a full-length, single ferritin polypeptide, or any portion of ferritin amino acid sequence that is capable of directing self-assembly of monomeric ferritin subunits into oligomers. Fusion proteins including ferritin amino acid sequences are described, for example, in U.S. Pat. No. 7,097,841. The amino acid sequences of monomeric ferritin subunits, or portions thereof, of any ferritin protein can be used to produce fusion proteins of the present disclosure, so long as the monomeric ferritin subunits are capable of self-assembling into an oligomer or a nanoparticle. Variations can be made in the amino acid sequence of a ferritin protein without affecting its ability to self-assemble into an oligomer or a nanoparticle. Such variations include insertion of amino acid residues, deletions of amino acid residues, or substitutions of amino acid residues. For example, the sequence of a monomeric ferritin subunit included in a fusion protein according to the embodiments of the present invention can be derived from a mammalian ferritin amino acid sequence, but be divergent enough from the naturally occurring sequence, such that, when administered as an immunogen to a mammalian subject of the species from which the mammalian ferritin amino acid sequence was derived, it does not result in the production of antibodies that react with the natural ferritin protein of the mammal.

A ferritin amino acid sequence may be derived from a bacterial ferritin protein, a plant ferritin protein, an algal ferritin protein, an insect ferritin protein, a fungal ferritin protein, and/or a mammalian ferritin protein. In some embodiments of fusion proteins of the present disclosure, ferritin amino acid sequence is derived from H. pylori. For example, a ferritin amino acid sequence included in a fusion protein as provided herein may be or may be derived from a sequence with at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO:7. As discussed above, fusion proteins according to the embodiments the present invention need not comprise a full-length sequence of a ferritin subunit polypeptide of H. pylori. Portions, or regions, of H. pylori ferritin subunit polypeptide can be used that contain an amino acid sequence directing self-assembly of monomeric ferritin subunits into oligomers. One example of such a region is located between amino acids 5 and 168 of the amino acid sequence H. pylori ferritin protein. More regions are described in Zhang 2011.

A ferritin amino acid sequence included in fusion proteins according to the embodiments of the present invention may include artificial glycosylation sites, for example, artificial (engineered)N-glycosylation sites, which are engineered by inserting artificial mutations into a ferritin amino acid sequence to create a consensus glycosylation sequence. For example, an artificial N-glycosylation site may be created by introducing a consensus sequence N—X-S/T (where X cannot be P) in a ferritin nucleic acid sequence. A consensus glycosylation sequence can be created by artificial substitutions of amino acid residues in a ferritin amino acid sequence. For example, an artificial N-glycosylation site in SEQ ID NO:7 can be created by introducing two amino acid substitutions: K to N at a position corresponding to position 75 of SEQ ID NO:7, and E to T at a position corresponding to position 75 of SEQ ID NO:7. In another example, an artificial N-glycosylation site in SEQ ID NO:7 can be created by introducing two amino acid substitutions: T to N at a position corresponding to position 67 of SEQ ID NO:7, and I to T at a position corresponding to position 69 of SEQ ID NO:7. In yet another example, an artificial N-glycosylation site in SEQ ID NO:7 can be created by introducing two amino acid substitutions: H to N at a position corresponding to position 74 of SEQ ID NO:7, and F to T at a position corresponding to position 76 of SEQ ID NO:7. In one more example, an artificial N-glycosylation site in SEQ ID NO:7 can be created by introducing two amino acid substitutions: E to N at a position corresponding to position 143 of SEQ ID NO:7, and H to T at a position corresponding to position 145 of SEQ ID NO:7.

Embodiments of fusion proteins according to the present invention include an amino acid sequence of an ebolavirus GP protein according to the present disclosure joined to at least 25 contiguous amino acids, at least 50 contiguous amino acids, at least 75 contiguous amino acids, at least 100 contiguous amino acids, or at least 150 contiguous amino acids of an amino acid sequence of a ferritin subunit polypeptide. In the embodiments of fusion proteins according to the present invention, an amino acid sequence of a ferritin subunit polypeptide is positioned after an amino acid sequence of an ebolavirus GP protein (i.e., downstream or C′ terminally relative to an ebolavirus GP protein amino acid sequence). Due to the presence of an amino acid sequence of a ferritin subunit polypeptide, fusion proteins according to the embodiments of the present invention assemble into nanoparticles, which are described in more detail elsewhere in the present disclosure. In some embodiments of a fusion protein, an amino acid sequence of an ebolavirus GP protein is joined to at least 25 contiguous amino acids, at least 50 contiguous amino acids, at least 75 contiguous amino acids, at least 100 contiguous amino acids, or at least 150 contiguous amino acids an amino acid sequence of a ferritin subunit polypeptide of H. pylori. An amino acid sequence of a ferritin subunit polypeptide of H. pylori that is included in a fusion protein according to the embodiments of the present invention can have at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO:7. An amino acid sequence of a ferritin subunit polypeptide of H. pylori that is included in a fusion protein according to the embodiments of the present invention results in a fusion protein that self-assembles into oligomers or nanoparticles.

In some embodiments of the fusion proteins according to the present invention, an amino acid sequence of an ebolavirus GP protein and an amino acid sequence of a ferritin subunit polypeptide are joined by a “linker” amino acid sequence. The peptide linker may be, for example, 2 to 5, 2 to 10, 2 to 20, 2 to 30, 2 to 40, 2 to 50, or 2 to 60, or more amino acids in length, for example, 2 amino acids, 3 amino acids, 4 amino acids, 5 amino acids, 10 amino acids, 15 amino acids, 25 amino acids, 35 amino acids, 45 amino acids, 50 amino acids, or 60 amino acids. Depending on length, linker sequence may have various conformations in secondary structure, such as helical, β-strand, coil/bend, and turns. In some instances, a linker sequence may have an extended conformation and function as an independent domain that does not interact with the adjacent protein domains. A linker sequence may be rigid or flexible. A flexible linker sequence may increase the range of orientations that may be adopted by the domains of the fusion protein. A rigid linker can be used to keep a fixed distance between the domains and to help maintain their independent functions. Linker sequences for fusion proteins are described, for example, in Chen et al. 2013. In some embodiments, a linker is or includes an amino acid sequence SGG, GSG, GG, GSGG (SEQ ID NO:12), NGTGGSG (SEQ ID NO:13), G, or GGGGS (SEQ ID NO:14). In an exemplary embodiment of a fusion protein, an artificially modified ebolavirus amino acid sequence with at least 80%, at least 85%, at least 90%, at least 91%, at least 92%, at least 93%, at least 94%, at least 95%, at least 96%, at least 97%, at least 98%, or at least 99%, or 100% sequence identity to with at least 80%, at least 85%, at least 90%, at least 91%, at least 92%, at least 93%, at least 94%, at least 95%, at least 96%, at least 97%, at least 98%, or at least 99%, or 100% sequence identity to amino acid residues of SEQ ID NO:12, SEQ ID NO:13, SEQ ID NO:14, or SEQ ID NO: 15. is joined to an amino acid sequence of a ferritin subunit polypeptide with at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO:7 by a linker with or including an amino acid sequence SGG, GSG, GG, GSGG (SEQ ID NO:12), NGTGGSG (SEQ ID NO:13), G, or GGGGS (SEQ ID NO:14).

Fusion proteins described in a present disclosure may include a domain or sequence useful for protein isolation. In some embodiments, the polypeptides comprise an affinity tag, for example an AviTag™ tag (GLNDIFEAQKIEWHE; SEQ ID NO:22), a Myc tag (EQKLISEEDL; SEQ ID NO:25), a polyhistidine tag (such as SEQ ID NO:23), an albumin-binding protein, an alkaline phosphatase, an AU1 epitope, an AU5 epitope, a biotin-carboxy carrier protein (BCCP), or a FLAG tag (DYKDDDDK; SEQ ID NO:26), to name a few. In some embodiments, the affinity tags are useful for protein isolation. See, for example, Kimple et al. 2013. In some embodiments, the polypeptides or proteins include a signal sequence useful for protein isolation, for example a mutated Interleukin-2 signal peptide sequence, which promotes secretion and facilitates protein isolation. See, for example, Low et al. 2013. In some embodiments, a fusion protein may include a protease recognition site, for example, TEV protease cut site, which may be useful for, among other things, removal of a signal peptide or affinity purification tag following fusion protein isolation.

Some embodiments of the fusion proteins described in the present disclosure may include a signal sequence, for example, in order to facilitate secretion of fusion proteins from cells after expression. For example, in some embodiments, an ebolavirus GP protein amino acid sequence may be preceded by a signal sequence. The signal sequence may immediately precede ebolavirus GP amino acid sequence, or there can be a linker or a spacer sequence between the signal sequence and the ebolavirus GP amino acid sequence.

Provided in this disclosure and included among the embodiments of the present invention are nanoparticles that include fusion proteins comprising an artificially modified amino acid sequence of an ebolavirus GP protein and an amino acid sequence of a ferritin subunit polypeptide. Due to the fact that fusion proteins according to the embodiments the present invention include an amino acid sequence of a ferritin subunit polypeptide, they can self-assemble into oligomers. An oligomeric structure, or supramolecule, resulting from such self-assembly is referred to as a nanoparticle. An exemplary embodiment of the present invention is a nanoparticle comprising an oligomer of a fusion protein, as described in the present disclosure.

Nanoparticles according to the embodiments of the present invention can contain 24 fusion protein subunits. Nanoparticles according to the embodiments of the present invention display at least a portion of an ebolavirus GP protein on their surface as trimers. In other words, a nanoparticle according to the embodiments of the present invention comprises surface-exposed trimers of an ebolavirus GP protein. A nanoparticle can include eight surface-exposed trimers of an ebolavirus GP protein. When the nanoparticle is administered to a subject, the surface-exposed trimers of an ebolavirus GP protein trimer are accessible to the immune system of the subject to and thus can elicit an immune response to an ebolavirus GP protein. Immunogenic nanoparticles composed of fusion proteins incorporating ferritin amino acid sequences are described, for example, in U.S. Pat. Nos. 9,441,19 and 10,137,190.

### Nucleic Acids, Vectors, Cells, and Related Methods

Provided in this disclosure and included among the embodiments of the present invention are nucleic acids encoding fusion proteins according to the embodiments of the present invention and described elsewhere in the present disclosure. Nucleic acids according to the embodiments of the present invention encode fusion proteins of an amino acid sequence of an ebolavirus GP protein and an amino acid sequence of a ferritin subunit polypeptide (which can be referred to simply as “ferritin”). Nucleic acids according to the embodiments of the present invention can be DNA or RNA. Nucleic acids described in the present disclosure can be used for producing fusion proteins and nanoparticles according to the embodiments of the present invention. For example, nucleic acids described in the present disclosure can be used for producing fusion proteins and nanoparticles according to the embodiments of the present invention in order to generate fusion proteins or nanoparticles to be used as immunogenic compositions, or vaccines, against ebolaviruses, such as, but not limited to, Bundibugyo ebolavirus (BDBV), Reston ebolavirus (RESTV), Sudan ebolavirus (SUDV), Taï Forest ebolavirus (TAFV, originally Côthe d'Ivoire ebolavirus), Zaire ebolavirus (Zaire, Ebola or EBOV), Bombali ebolavirus (BOMV), or any combination thereof. In another example, nucleic acids described in the present disclosure can be used as nucleic acid vaccines, which are administered to subjects for the purpose of producing in subject fusion proteins and nanoparticles according to the embodiments of the present invention, in order to elicit in the subjects protective immune response against an ebolavirus, including, but not limited to, Bundibugyo ebolavirus (BDBV), Reston ebolavirus (RESTV), Sudan ebolavirus (SUDV), Taï Forest ebolavirus (TAFV, originally Côthe d'Ivoire ebolavirus), Zaire ebolavirus (Zaire, Ebola or EBOV), or Bombali ebolavirus (BOMV), or any combination thereof. Methods of using nucleic acids according to the embodiments of the present invention are described elsewhere in the present disclosure.

Embodiments of nucleic acids encoding fusion proteins described in the present disclosure encode fusion proteins including an artificially modified amino acid sequence of an ebolavirus GP protein according to the present disclosure (for example, an amino acid sequence with at least 80%, at least 85%, at least 90%, at least 91%, at least 92%, at least 93%, at least 94%, at least 95%, at least 96%, at least 97%, at least 98%, or at least 99%, or 100% sequence identity to identity to SEQ ID NO: 12, SEQ ID NO:13, SEQ ID NO:14, or SEQ ID NO:15) joined (directly or via a linker) to at least 25 contiguous amino acids, at least 50 contiguous amino acids, at least 75 contiguous amino acids, at least 100 contiguous amino acids, or at least 150 contiguous amino acids an amino acid sequence of a ferritin subunit polypeptide. Some embodiments of nucleic acids encoding fusion proteins described in the present disclosure encode fusion proteins in which an artificially modified amino acid sequence of an ebolavirus GP protein (for example, an amino acid sequence with at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO:12, SEQ ID NO:13, SEQ ID NO: 14, or SEQ ID NO:15) is joined (directly or via a linker) to at least 25 contiguous amino acids, at least 50 contiguous amino acids, at least 75 contiguous amino acids, at least 100 contiguous amino acids, or at least 150 contiguous amino acids of a ferritin subunit polypeptide of H. pylori, such as an amino acid sequence with at least 80%, at least 85%, at least 90%, at least 91%, at least 92%, at least 93%, at least 94%, at least 95%, at least 97%, at least 98%, or at least 99%, or 100% sequence identity to SEQ ID NO:7. Some examples of nucleic acids described in the present disclosure encode fusion proteins having amino acid sequences with at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO:8, SEQ ID NO:9, SEQ ID NO:10, or SEQ ID NO:11.

Also provided in this disclosure and included among the embodiments of the present invention are nucleic acid constructs that include the nucleic acid sequences provided herein. Some embodiments of the nucleic acid constructs are purified nucleic acid molecules encoding fusion proteins according to the embodiments of the present invention. For example, a nucleic acid construct can be an engineered (recombinant) DNA nucleic acid sequence comprising a promoter operably linked to a nucleic acid encoding a fusion protein according to an embodiment of the present invention. A nucleic acid sequence is “operably linked” when it is placed into a functional relationship with another nucleic acid sequence. A promoter is a region or a sequence located upstream and/or downstream from the start of transcription that is involved in recognition and binding of RNA polymerase and other proteins to initiate transcription. A promoter is generally a nucleic acid sequence or sequences that function when in a relatively fixed location in regard to the transcription start site. A promoter contains core elements required for basic interaction of RNA polymerase and transcription factors, and may contain upstream elements and response elements. A promoter included in nucleic acid constructs according to embodiments of the present invention can be a eukaryotic or a prokaryotic promoter. In some embodiments, the promoter is an inducible promoter. In some embodiments, the promoter is a constitutive promoter. A promoter included in a nucleic acid construct according to the embodiments of the present invention is capable of directing or driving expression of nucleic acid sequence encoding a fusion protein described in the present disclosure in a host cell or host organism of interest. For preparing nucleic acid constructs according to the embodiments of the present invention, nucleic acids may be manipulated, so as to provide for the nucleic acid sequences in the proper orientation and, as appropriate, in the proper reading frame. Toward this end, adapters or linkers may be employed to join the nucleic acid fragments or other manipulations may be involved to provide for convenient restriction sites, removal of superfluous nucleic acid sequences, removal of restriction sites, etc. For this purpose, in vitro mutagenesis, primer repair, restriction, annealing, resubstitutions, such as transitions and transversions, may be involved.

A nucleic acid according to the embodiments of the present invention can be included in an expression cassette for expression of a fusion protein encoded by the nucleic acid in a host cell or an organism of interest. In some embodiments, a nucleic acid according to the embodiments of the present invention can be codon-optimized for expression in a host cell or an organism of interest. An expression cassette can include 5′ and 3′ regulatory sequences operably linked to the nucleic acid encoding a fusion protein according to an embodiment of the present invention. An expression cassette can also include nucleic acid sequences encoding other polypeptides or proteins. An expression cassette can include a plurality of restriction sites and/or recombination sites for insertion of various nucleic acid sequences into the expression cassette and/or for insertion of the expression cassette into other nucleic acids, such as vectors. An expression cassette can include various regulatory regions or sequences, such as, but are not limited to, transcriptional initiation start sites, operators, activators, enhancers, other regulatory elements, ribosomal binding sites, initiation codons, termination signals, and the like. Exemplary regulatory sequences included in the expression cassettes are promoters, transcriptional regulatory regions, and/or translational termination regions, which may be endogenous or heterologous to the host cell or host organism, or to each other. In this context, “heterologous” means a nucleic acid sequence that does not originate in the host cell or host organism, or is substantially modified from its form occurring in the host cell or host organism. An expression cassette can also include one or more selectable marker genes for the selection of host cells containing the expression cassette. Marker genes include, but are not limited to, genes conferring antibiotic resistance, such as those conferring hygromycin resistance, ampicillin resistance, gentamicin resistance, neomycin resistance, to name a few. Additional selectable markers are known, and any can be used. An exemplary expression cassette can include, in the 5′ to 3′ direction, a transcriptional and translational initiation region (including a promoter), a nucleic acid sequence encoding a fusion protein described in the present disclosure, and transcriptional and translational termination regions functional in the host cell or host organism of interest.

Also included among the embodiments of the present invention are vectors including nucleic acids or nucleic acid constructs according to the embodiments of the present invention. Such vectors can include necessary functional elements that direct and regulate transcription of the nucleic acid sequences included in the vector. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers that can serve to select for cells containing the vector or the vector containing the insert, RNA splice junctions, a transcription termination region, or any other region that may serve to facilitate the expression of the inserted gene or hybrid. The vector, for example, can be a plasmid.

A vector according to the embodiments of the present invention can be a bacterial vector, such as a bacterial expression vector. For example, a vector based on one of numerous E. coli expression vectors can be useful for the expression of a nucleic acid according to the embodiments of the present invention. Other bacterial hosts suitable for expression of nucleic acids according to the embodiments of the present invention include bacilli, such as Bacillus subtilis, and other Enterobacteriaceae, such as Salmonella, Senatia, and various Pseudomonas species. In these prokaryotic hosts, one can also use suitable expression vectors, which will typically contain expression control sequences compatible with the host cell (such as an origin of replication). Any number of a variety of well-known promoters can be used in bacterial expression vectors, such as a lactose promoter system, a tryptophan (Trp) promoter system, a beta-lactamase promoter system, or a promoter system from phage lambda.

Eukaryotic cells, including, but not limited to, yeast cells, mammalian cells and insect cells, also permit the expression of proteins in an environment that favors important post-translational modifications such as folding and cysteine pairing, addition of complex carbohydrate structures, and secretion of active protein. Accordingly, vectors useful for the expression of nucleic acids described in the present disclosure in yeast cells, mammalian cells and insect cells are also envisioned and included among the embodiments of the present invention. A vector according to the embodiments of the present invention can be a yeast expression vector suitable for expression of a nucleic acid according to the embodiments of the present invention in yeast cells, such as, but not limited to, cells of Pichia pastoris or Saccharomyces cerevisiae. Expression vectors used in eukaryotic cells may contain sequences necessary for the termination of transcription. These regions are transcribed as polyadenylated segments in the untranslated portion of the mRNA. Accordingly, a transcription unit included in an eukaryotic expression vector may contain a polyadenylation region. One benefit of this region is that it increases the likelihood that the transcribed unit will be processed and transported like mRNA. The 3′ untranslated regions also include transcription termination sites. Expression vectors for eukaryotic cells can include expression control sequences, such as enhancers, and necessary information processing sites, such as ribosome binding sites, RNA splice sites etc.

Expression vectors according to the embodiments of the present invention can also include nucleic acids described in the present disclosure under the control of an inducible promoter such as the tetracycline inducible promoter or a glucocorticoid inducible promoter. The nucleic acids of the present invention can also be under the control of a tissue-specific promoter to promote expression of the nucleic acid in specific cells, tissues or organs. Any regulatable promoter, such as a metallothionein promoter, a heat-shock promoter, and other regulatable promoters are also contemplated. Furthermore, a Cre-loxP inducible system can also be used, as well as a Flp recombinase inducible promoter system.

In some embodiments, a nucleic acid encoding a fusion protein according to the embodiments of the present invention may be incorporated into a viral vector for delivery into a host cell or host organism. Accordingly, the vectors according to the embodiments of the present invention include viral vectors that transport the nucleic acids encoding fusion proteins described in the present disclosure into cells without degradation and include a promoter yielding expression of the nucleic acids in the cells into which it is delivered. Suitable viral vectors include adenovirus vectors, adeno-associated viral (AAV) vectors, herpes viral vectors, retroviral vectors, poxviral vectors, or lentiviral vectors. Methods of constructing and using such vectors are well known. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. The necessary functions of the removed early genes are typically supplied by cell lines that have been engineered to express the gene products of the early genes in trans.

For example, recombinant viruses in the pox family of viruses can be used as vectors for delivering the nucleic acid molecules according to the embodiments of the present invention into a host cell or host organism. These include vaccinia viruses and avian poxviruses, such as the fowlpox and canarypox viruses. Methods for producing recombinant pox viruses are known. Representative examples of recombinant pox viruses include ALVAC, TROVAC, and NYVAC. In another example, adenovirus vectors can be used for delivering the nucleic acid molecules according to the embodiments of the present invention into a host cell or host organism. In one more example, adeno-associated virus (AAV) vector systems can be used for delivering the nucleic acid molecules according to the embodiments of the present invention into a host cell or host organism. In one more example, retroviral vectors can be used for delivering the nucleic acid molecules according to the embodiments of the present invention into a host cell or host organism. Examples of retroviral vectors include, but are not limited to, vectors based on Murine Maloney Leukemia virus (MMLV), and retroviruses that express the desirable properties of MMLV as a vector. In yet another example, molecular conjugate vectors, such as the adenovirus chimeric vectors can be used for delivering nucleic acid molecules according to the embodiments of the present invention into a host cell or host organism. Vectors derived from the members of the Alphavirus genus, such as, but not limited to, Sindbis, Semliki Forest, and Venezuelan Equine Encephalitis viruses, can also be used for delivering nucleic acid molecules according to the embodiments of the present invention into a host cell or host organism.

In some embodiments, a nucleic acid encoding a fusion protein according to the embodiments of the present invention may be incorporated into a transposon vector, which can be used as a part of a transposon vector system to integrate the nucleic acid encoding the fusion protein into transcriptionally active sites of genome of a host cell. Transposon vector systems are discussed, for example, in Wei et al. 2022. Accordingly, the vectors according to the embodiments of the present invention include transposon vectors that transport the nucleic acids encoding fusion proteins described in the present disclosure into cells. A non-limiting example of a transposon vector system is a system that includes Leap-In Transposase® available from ATUM (Newark, California) and its accompanying suite of transposon vectors. To use the above transposon vector system, transposase mRNA is co-transfected with the transposon vector encoding a protein of interest (for example, a fusion protein according to the embodiments of the present invention) into a cell. After the mRNA is translated in the cell, the transposase protein transiently acts to integrate the transposon into the cell genome. The transposase mRNA is degraded through the normal cellular RNA turnover pathways. Pools of cells with the stably integrated DNA encoding the protein of interest can then be recovered, and individual cells can be cloned, thereby creating stable cell lines encoding the protein of interest, such as a fusion protein according to the embodiments of the present invention.

Also provided in this disclosure and included among the embodiments of the present invention are cells comprising a nucleic acid, a nucleic acid construct, or a vector according to the embodiments of the present invention. Such cells can be referred to as “host cells” (or “host cell,” in singular). Some host cells can produce fusion proteins described in the present disclosure, while other host cells may be used for producing or maintaining nucleic acids, DNA constructs, or vectors according to the embodiments of the present invention. A host cell can be an in vitro, ex vivo, or in vivo host cell. Populations of any of the host cells and cell cultures comprising one or more host cells are also included among the embodiments of the present invention. The host cell can be a prokaryotic cell, including, for example, a bacterial cell. Alternatively, the cell can be a eukaryotic cell. Examples of prokaryotic host cells are cells of E. coli, Pseudomonas, Bacillus or Streptomyces. Examples of eukaryotic cells are yeast cells (such as cells of Saccharomyces yeast, or methylotrophic yeast such as Pichia, Candida, Hansenula, and Torulopsis); animal cells, such as CHO, R1. 1, B—W and LM cells, African Green Monkey kidney cells (for example, COS 1, COS 7, BSC1, BSC40, and BMT10), insect cells (for example, Sf9), human cells (such as human embryonic kidney cells, for instance, HEK293, or HeLa cells). In an exemplary embodiment, a cell is a mammalian cell, such as a CHO cell or an Expi-293F, with stably integrated into genome one or more nucleic acid sequences encoding a fusion protein according to the present disclosure.

Methods of producing or generating host cells (meaning cells comprising a nucleic acid, a nucleic acid construct, or a vector according to the embodiments of the present invention) are also included among the embodiments of the present invention. A nucleic acid, a nucleic acid construct, or a vector according to the embodiments of the present invention can be transferred or introduced into the host cell by well-known methods, which vary depending on the type of the host cell. The “introducing” and the related terms or phrases used in the context of introducing a nucleic acid a nucleic acid construct, or a vector into a cell refers to the translocation of the nucleic acid sequence from outside a cell to inside the cell. In some cases, introducing refers to translocation of the nucleic acid from outside the cell to inside the nucleus of a eukaryotic cell. Various methods of such translocation are contemplated, including but not limited to, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease (CRISPR-Cas9), a transcription activator-like effector nuclease (TALEN), a zinc finger nuclease (ZFN), or a megaTAL (MT) can also be used to introduce a nucleic acid into a cell.

Methods of producing or generating fusion proteins and nanoparticles described in the present disclosure are also included among the embodiments of the present invention. An exemplary method of producing the fusion protein or a nanoparticle can include a step of introducing into a cell a nucleic acid according to an embodiment of the present invention, a nucleic acid construct according to an embodiment of the present invention, or a vector according to an embodiment of the present invention. The introducing step is carried out as described elsewhere in the present disclosure, and, as an outcome of such step, a cell (which can be referred to as “a host cell”) comprising the nucleic acid, the nucleic acid construct or the vector is generated. An exemplary method of producing the fusion protein can include a step of incubating the host cell under conditions allowing for expression of a fusion protein. An exemplary method of producing the nanoparticle can include a step of incubating the host cell under conditions allowing for expression of a fusion protein and self-assembly of the nanoparticle. After expression in the host cell, a fusion protein or a nanoparticle can be isolated or purified using various purification methods. In some embodiments, the fusion protein can be isolated from the host cell and allowed to self-assemble into nanoparticles in vitro.

In one example illustrating a process of producing or generating fusion proteins and nanoparticles described in the present disclosure, a nucleic acid or a nucleic acid construct encoding a fusion protein according to an embodiment of the present invention is introduced into a plasmid or other vector, which is then used to transform living cells. For instance, a nucleic acid encoding a fusion protein according to an embodiment of the present invention is inserted in a correct orientation into an expression vector that provides the necessary regulatory regions, such as promoters, enhancers, poly A sites and other sequences. In some cases, it may be desirable to express the fusion protein under the control of an inducible or tissue-specific promoter. The expression vector may then be transfected into living cells using various methods, such as lipofection or electroporation, thus generating host cells expressing the fusion protein. The cells expressing the fusion protein may be selected by appropriate antibiotic selection or other methods and cultured. Larger amounts of the fusion protein may be produced by growing the cells in commercially available bioreactors. Once expressed by the host cells, the fusion protein may be isolated (purified) according to standard procedures, such as dialysis, filtration, and chromatography. A step of lysing the cells to isolate the fusion protein can be included. Thus, a method of producing or generating a fusion protein according to an embodiment of the present invention may contain one or more steps of culturing a cell comprising a vector under conditions permitting expression of the fusion protein, harvesting the cells and/or harvesting the medium from the cultured cells, and isolating the fusion protein from the cells and/or the culture medium. Compositions, methods, and kits related to the production of fusion proteins described in the present disclosure are included within the scope of the embodiments of the present invention.

### Immunogenic Compositions and Kits

Immunogenic compositions containing any of the fusion proteins described in the present disclosure, nanoparticle described in the present disclosure, nucleic acids described in the present disclosure, nucleic acids constructs described in the present disclosure, or vectors described in the present disclosure are included among the embodiments of the present invention. Immunogenic compositions according to the embodiments of the present invention can be also referred to as “vaccines.” An immunogenic composition may contain a fusion protein, a nanoparticle, a nucleic acid, a nucleic acids construct, or a vector according to the present invention and a pharmaceutically acceptable carrier (excipient). An immunogenic composition may contain a fusion protein, a nanoparticle, a nucleic acid, a nucleic acids construct, or a vector according to the embodiments of the present invention and an adjuvant. An immunogenic composition may contain a fusion protein, a nanoparticle, a nucleic acid, a nucleic acids construct, or a vector according to the embodiments of the present invention and other components, such as, but not limited to, a diluent, solubilizer, emulsifier, or preservative. An immunogenic composition according to the present invention may be a solution, such as an aqueous solution, a suspension, such as an aqueous suspension, or may be in dry form, such as in lyophilized form. Some of the components (or ingredients) included in immunogenic compositions in addition to a fusion protein, a nanoparticle, a nucleic acid, a nucleic acids construct, or a vector according to the embodiments of the present invention are described in more detail elsewhere in the present disclosure.

Immunogenic compositions containing any of the fusion proteins described in the present disclosure, nanoparticle described in the present disclosure, nucleic acids described in the present disclosure, nucleic acids constructs described in the present disclosure, or vectors described in the present disclosure are included among the embodiments of the present invention. Immunogenic compositions according to the embodiments of the present invention can be also referred to as “vaccines.” An immunogenic composition may contain a fusion protein, a nanoparticle, a nucleic acid, a nucleic acids construct, or a vector according to the present invention and a pharmaceutically acceptable carrier (excipient). An immunogenic composition may contain a fusion protein, a nanoparticle, a nucleic acid, a nucleic acids construct, or a vector according to the embodiments of the present invention and an adjuvant. An immunogenic composition may contain a fusion protein, a nanoparticle, a nucleic acid, a nucleic acids construct, or a vector according to the embodiments of the present invention and other components, such as, but not limited to, a diluent, solubilizer, emulsifier, or preservative. An immunogenic composition according to the present invention may be a solution, such as an aqueous solution, a suspension, such as an aqueous suspension, or may be in dry form, such as in lyophilized form. Some of the components (or ingredients) included in immunogenic compositions in addition to a fusion protein, a nanoparticle, a nucleic acid, a nucleic acids construct, or a vector according to the embodiments of the present invention are described in more detail elsewhere in the present disclosure.

Some embodiments of the immunogenic compositions contain one or more fusion proteins or nucleic acids encoding the fusion proteins described elsewhere in the present disclosure. For example, an immunogenic composition may contain two or more, three or more, four or more, five or more etc. different fusion proteins described elsewhere in the present disclosure. In another example, an immunogenic composition may contain nucleic acids encoding two or more, three or more, four or more, five or more etc. different fusion proteins described elsewhere in the present disclosure. The nucleic acids encoding two or more, three or more, four or more, five or more etc. different fusion may be included in the same nucleic acid construct, such as a vector, or in different nucleic acid constructs. For example, an immunogenic composition can contain one or more, two or more, three or more, four or more, five or more etc. of fusion proteins or nucleic acids encoding fusion proteins having amino acid sequences that have at least 80%, at least 85%, at least 90%, at least 91%, at least 92%, at least 93%, at least 94%, at least 95%, at least 96%, at least 97%, at least 98%, or at least 99%, or 100% sequence identity to SEQ ID NO:8, SEQ ID NO:9, SEQ ID NO: 10, or SEQ ID NO:11.

An immunogenic composition according to the embodiments of the present invention can include a pharmaceutically acceptable carrier or excipient. A pharmaceutically acceptable carrier or excipient is a material that is not biologically or otherwise undesirable, meaning the material that can be administered to a subject without causing undesirable biological effects or interacting in a deleterious manner with the other components of the pharmaceutical composition in which it is contained. The carrier or excipient is typically selected to minimize degradation of other ingredients of the composition in which the carrier or the excipient is included, and to minimize adverse side effects (such as allergic side effects) in the subject. Examples of aqueous pharmaceutically acceptable carriers include, but are not limited to, sterile water, saline, buffered solutions like Ringer's solution, glycerol solutions, ethanol, dextrose solutions, allantoic fluid, or combinations of the foregoing. The pH of the aqueous carriers is generally about 5 to about 8 or from about 7 to 7.5. A carrier may include a pH controlling buffer. The preparation of such aqueous carriers insuring sterility, pH, isotonicity, and stability is effected according to established protocols. Examples of non-aqueous carriers are propylene glycol, polyethylene glycol, vegetable oils such as olive oil, and injectable organic esters such as ethyl oleate. Other exemplary carriers sustained release preparations, such as semipermeable matrices of solid hydrophobic polymers. Other exemplary carriers are matrices in the form of shaped articles, such as, but not limited to, films, liposomes, or microparticles. Certain carriers may be more preferable depending upon, for instance, the route of administration and concentration of composition being administered.

An immunogenic composition according to the embodiments of the present invention can include an adjuvant. Some examples of chemical adjuvants are aluminum phosphate, benzyalkonium chloride, ubenimex, QS21, aluminium hydroxide (such as alum, an aluminum hydroxide wet gel suspension, for example, Alhydrogel® (Croda International, UK)), saponins (for example, Quil-A® (Croda International, UK)), squalenes (for example, Adda Vax™). Quil-AR is a commercially available, highly purified and concentrated saponin adjuvant purified from bark extract of the Quillaja saponaria (Molina) tree that has been specifically developed for use as an adjuvant. It comprises a complex mixture of approximately 25 different saponin molecules which have the triterpenoid backbone in common. Some examples of the so-called “genetic” adjuvants are IL-2 gene or its fragments, granulocyte macrophage colony-stimulating factor (GM-CSF) gene or fragments thereof, IL-18 gene or fragments thereof, chemokine (C—C motif) ligand 21 (CCL21) gene or fragments thereof, IL-6 gene or fragments thereof, CpG, LPS, TLR agonists (for example, Monophosphoryl Lipid A (MPLA)), and other immune stimulatory genes. Some examples of protein adjuvants are IL-2 or fragments thereof, granulocyte macrophage colony-stimulating factor (GM-CSF) or fragments thereof, IL-18 or its fragments, chemokine (C—C motif) ligand 21 (CCL21) or fragments thereof, IL-6 or fragments thereof, CpG, LPS, TLR agonists and other immune stimulatory cytokines or their fragments. Some examples of lipid adjuvants are cationic liposomes, N3 (cationic lipid), MPLA, Quil-A®, and AddaVax™. Other exemplary adjuvants include, but are not limited to, cholera toxin, enterotoxin, Fms-like tyrosine kinase-3 ligand (Flt-3L), bupivacaine, marcaine, and levamisole. In some embodiments, the immunogenic composition comprises Quil-A®. In some embodiments, the immunogenic composition comprises alum. In some embodiments, the immunogenic composition comprises CpG. More than one adjuvant may be included in immunogenic compositions according to the embodiments of the present invention. For example, in some embodiments, the immunogenic composition can comprise alum and CpG. One exemplary embodiment of the present invention is an immunogenic composition comprising a fusion protein of an artificially modified amino acid sequence of an ebolavirus GP protein and an amino acid sequence of a ferritin subunit polypeptide, as described in the present disclosure, and alum as an adjuvant. Alum may be obtained from any suitable source. The inventors discovered that including alum in immunogenic compositions according to the embodiments of the present invention improves immunogenic properties of such compositions. Accordingly, it is advantageous to use alum as an adjuvant, due to alum's low cost and easy availability.

Immunogenic compositions according to the embodiments of the present invention are generally formulated to be nontoxic or minimally toxic to subject at the dosages and concentrations used for administration. In some embodiments, a formulation of an immunogenic composition may include an appropriate amount of a pharmaceutically acceptable salt to render the formulation isotonic. In some embodiments, a formulation of an immunogenic composition may include components for modifying, maintaining, or preserving, for example, the pH, osmolality, viscosity, clarity, color, isotonicity, odor, sterility, stability, rate of dissolution or release, adsorption or penetration of the composition. A formulation of an immunogenic composition may include one or more of the following components: amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, polyethylene glycol (PEG), sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); and/or delivery vehicles.

In some embodiments, an immunogenic composition can be prepared in a dry form (i.e., dehydrated form), such as a lyophilized form. Such a formulation can be referred to as “lyophilized” or a “lyophilizate.” Lyophilization is a process of or freeze-drying, during which a solvent is removed from a liquid formulation. Lyophilization process may include one or more of simultaneous or sequential steps of freezing and drying. Immunogenic compositions according to the embodiments of the present invention can be lyophilized in an aqueous solution comprising a nonvolatile or volatile buffer. Non-limiting examples of suitable nonvolatile buffers are PBS, Tris-HCl, HEPES, or L-Histidine buffer. Non-limiting examples of suitable volatile buffers are ammonium bicarbonate, Ammonia/acetic acid, or N-ethylmorpholine/acetate buffer. A lyophilized immunogenic composition according to the embodiments of the present invention can include appropriate carriers or excipients. Such appropriate excipients may include, but are not limited to, a cryo-preservative, a bulking agent, a surfactant, or their combinations. Exemplary excipients include one or more of a polyol, a disaccharide, or a polysaccharide, such as, for example, mannitol, sorbitol, sucrose, trehalose, and/or dextran 40. In some instances, the cryo-preservative may be sucrose and/or trehalose. In some instances, the bulking agent may be glycine or mannitol. In one example, the surfactant may be a polysorbate such as, for example, polysorbate-20 and/or polysorbate-80. A lyophilized immunogenic composition according to the embodiments of the present invention can be, for example, in a cake or powder form. Lyophilized immunogenic compositions may be rehydrated/solubilized/reconstituted in a carrier or excipient (e.g., water or buffer solution) prior to use. Some embodiments of the immunogenic compositions are reconstituted in a water or buffer solution comprising sucrose.

An immunogenic composition according to embodiments of the present invention can be sterile prior to administration to a subject. Sterilization can be accomplished by filtration through sterile filtration membranes. When the immunogenic composition is lyophilized, sterilization can be conducted either prior to or following lyophilization and reconstitution. An immunogenic composition can be stored in sterile containers, such as vials or bags, as a solution, suspension, gel, emulsion, solid, or as a dehydrated or lyophilized powder.

Kits including immunogenic compositions described in the present disclosure are also included among the embodiments of the present invention. For example, a kit may include an immunogenic composition and a container for its storage, such as a bag or a vial. Such a container may have a sterile access port, for example, a bag or vial having a stopper pierceable by a hypodermic injection needle. In another example, a kit may include an immunogenic composition in lyophilized or concentrated form and diluent. In such a kit, a diluent may also be a pharmaceutically acceptable carrier or excipient, as described elsewhere in the present disclosure. Examples of diluents that may be included in such a kit are saline, buffered saline, water, or sucrose. In another example, a kit may include an immunogenic composition and a device for administering the immunogenic composition. A device for administering the composition may be a syringe for injection or oral administration (for example, the kit may be a syringe pre-filled with a liquid immunogenic composition), a microneedle device, such as a microneedle patch, an inhaler, or a nebulizer. In some embodiments, a kit may contain a defined amount of an immunogenic composition capable of eliciting a protective immune response against an ebolavirus in a subject, when administered as a single dose. In some embodiments, a kit may contain multiple doses of a defined amount of an immunogenic composition capable of eliciting a protective immune response against an ebolavirus in a subject. For example, a kit may contain multiple vials, syringes or microneedle patches containing an immunogenic composition.

### Methods of Inducing an Immune Response

Methods of inducing or eliciting an immune response against an ebolavirus in a subject by administering to the subject an immunogenic composition described in the present disclosure are included among the embodiments of the present invention. In embodiments of such methods, an immunogenic composition is administered in an amount capable of inducing or eliciting a protective immune response against an ebolavirus in the subject. A protective immune response against an ebolavirus in the subject may include production of anti-ebolavirus neutralizing antibodies in the subject. An amount of the immunogenic composition capable of inducing or eliciting a protective immune response against an ebolavirus in the subject can be described as an “effective amount” or “immunologically effective amount,” and may be administered as one dose or as two or more doses. Effective amounts and schedules for administration may be determined empirically.

Dosage ranges for administration of the immunogenic compositions described in the present disclosure are those large enough to produce the desired effect—i.e., eliciting a protective immune response against an ebolavirus, such as, but not limited to, EBOV (Ebola or Zaire), BDBV (Bundibugyo), RESTV (Reston), SUDV (Sudan), TAFV (Taï Forest), or Bombali (BOMV) ebolavirus, or any combination thereof. The dosage should not be so large as to cause substantial adverse side effects, such as unwanted cross-reactions, anaphylactic reactions, and the like. Generally, the dosage may vary with the age, condition, sex, medical status, route of administration, or whether other drugs are included in the regimen. The dosage can be adjusted by a medical professional in the event of any contraindications. Dosages can vary, and the agent can be administered in one or more dose administrations daily, for one or several days, including a prime and boost paradigm.

When used in the context of methods of inducing or eliciting a protective immune response against an ebolavirus in a subject, immunogenic compositions described in the present disclosure can be administered via any of several routes of administration, including, but not limited to, orally, parenterally, intravenously, intramuscularly, subcutaneously, transdermally, by nebulization/inhalation, or by installation via bronchoscopy. An immunogenic composition can be administered by oral inhalation, nasal inhalation, or intranasal mucosal administration. Administration of the immunogenic compositions described in the present disclosure by inhalant can be through the nose or mouth via delivery by spraying or droplet mechanism, for example, in the form of an aerosol. A form of administration may be chosen to optimize a protective immune response against an ebolavirus in a subject.

In the provided methods in which the immunogenic composition comprises a nucleic acid, a nucleic acid construct, or a vector according to the embodiments of the present invention (such a composition may be termed a “nucleic acid immunogenic composition” or a “nucleic acid vaccine”), the immunogenic composition can be introduced into the cells of the subject. Examples of nucleic acid delivery technologies include “naked DNA” facilitated (bupivacaine, polymers, peptide-mediated) delivery, and cationic lipid complexes or liposomes. The nucleic acids can be administered using ballistic delivery as described, for instance, in U.S. Pat. No. 5,204,253 or pressure (see, for example, U.S. Pat. No. 5,922,687). In some examples, particles comprised solely or mostly of a nucleic acid, a nucleic acid construct, or a vector according to the embodiments of the present invention can be administered to the subject. In some examples, a nucleic acid, a nucleic acid construct, or a vector according to the embodiments of the present invention can be adhered to particles, such as gold particles, for administration to the subject. When an immunogenic composition includes a viral vector, the viral vector can be introduced into cells obtained from the subject (autologous cells) and the cells can be administered to the subject. In some embodiments, an immunogenic composition comprising a nucleic acid, a nucleic acid construct, or a vector according to the embodiments of the present invention can be administered by injection or electroporation, or a combination of injection and electroporation.

In the context of the methods described in the present disclosure, a subject may be healthy and without higher risk of contracting an ebolavirus invention than the general public. In some instances, the subject can have an elevated risk of developing an ebolavirus infection, such that they are predisposed to contracting an ebolavirus infection, for example, due to exposure to ebolavirus infection, such as, but not limited to, in a medical setting, or due to living in or visiting an area where ebolavirus infections are endemic. A subject may also be a subject with a current ebolavirus infection and may have one or more than one symptom of the infection. A subject currently with an ebolavirus infection may have been diagnosed with an ebolavirus infection based on the symptoms or the results of diagnostic test.

The methods according to the embodiments of the present invention are useful for both prophylactic and therapeutic purposes. Methods of treating or preventing an ebolavirus infection in a subject, which include administering to a subject with an ebolavirus infection or susceptible to an ebolavirus infection an effective dose an immunogenic composition described in the present disclosure are also included among the embodiments of the present invention. In the methods according to the embodiments of the present invention, an immunogenic composition can be used alone or in combination with one or more therapeutic agents such as, for example, antiviral compounds for the treatment of an ebolavirus infection or disease. For prophylactic use, an effective amount of an immunogenic compositions described in the present disclosure can be administered to a subject prior to onset of an ebolavirus infection (for example, before obvious signs of infection) or during early onset (for example, upon initial signs and symptoms of infection). Prophylactic administration can occur at several days to years prior to the manifestation of symptoms of an ebolavirus infection. Prophylactic administration can be used, for example, in the preventative treatment of subjects identified as being predisposed to an ebolavirus infection. Therapeutic treatment involves administering to a subject a therapeutically effective amount of an immunogenic composition described in the present disclosure after diagnosis or development of infection.

In the context of the embodiments of the present invention, the terms “treatment,” “treat,” “treating” and the related terms and expressions refer to reducing one or more of the effects of an ebolavirus infection or one or more symptoms of an ebolavirus infection by eliciting an immune response in the subject. Thus, in the disclosed method, treatment can refer to a 10%, 20%, 30%, 40%, 50%, 60%, 70%, 80%, 90%, or 100% reduction in the severity of an established ebolavirus infection or a symptom of the ebolavirus infection. For example, a method for treating an ebolavirus infection is considered to be a treatment if there is a 10% reduction in one or more symptoms of the ebolavirus infection in a subject, as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of an ebolavirus infection or disease or symptoms of an ebolavirus infection or disease.

In the context of the embodiments of the present invention, the terms “prevent,” “preventing,” “prevention” of an ebolavirus infection or disease, and the related terms and expressions, refer to an action, for example, administration of an immunogenic composition that occurs before or at about the same time a subject begins to show one or more symptoms of an ebolavirus infection, which inhibits or delays onset or exacerbation or delays recurrence of one or more symptoms of the infection. As used in the present disclosure, references to decreasing, reducing, or inhibiting include a change of 10%, 20%, 30%, 40%, 50%, 60%, 70%, 80%, 90% or greater as compared to a control level. For example, the methods described in the present disclose can be considered to effect prevention of an ebolavirus infection, if there is about a 10% reduction in onset, exacerbation or recurrence of an ebolavirus infection, or symptoms of infection in a subject exposed to an ebolavirus to whom an immunogenic composition described in the present disclosure was administered, when compared to control subjects exposed to an ebolavirus that did not receive a composition for decreasing infection. Thus, the reduction in onset, exacerbation or recurrence of an ebolavirus infection can be about a 10, 20, 30, 40, 50, 60, 70, 80, 90, 100%, or any amount of reduction in between as compared to control subjects.

### EXAMPLES

The following examples are offered to illustrate, but not to limit the claimed invention.

### Example 1: Materials and Methods

### A. Cell Lines.

HEK-293T cells were purchased from American type culture collection (ATCC) and maintained in D10 medium-Dulbecco's Modified Eagle Medium (DMEM, Cytiva™ (Marlborough, Massachusetts) supplemented with 10% fetal bovine serum (GeminiBio, Sacramento, California) and 1% L-glutamine/penicillin/streptomycin (GeminiBio). Expi-293F cells were maintained in Freestyle293/Expi-293 media (2:1, v/v, Thermo Fisher Scientific, Waltham, Massachusetts) in polycarbonate shaking flasks (TriForest Enterprises, Inc., Irvine, California).

### B. Antibodies.

Monoclonal antibodies against Ebola glycoprotein (REGN3471, FVM04, REGN3470, ADI-15742, KZ52, and ADI-16061) were expressed in Expi-293F cells via transient transfection. Goat anti-mouse IgG, HRP conjugated (BioLegend, San Diego, California, 405306) or rabbit anti-human IgG, HRP conjugated (Abcam, Cambridge, United Kingdom ab6759) were used as secondary antibodies for Western blots or enzyme-linked immunosorbent assays (ELISAs).

### C. Antigen and Antibody Cloning.

DNA encoding EBOV GP (Mayinga, Zaire, 1976) ectodomain with the mucin-like domain deleted (residues 1-308, 490-656) and the transmembrane domain replaced with a GCN4 (Misasi et al. 2016), followed by an AviTag™ tag (GLNDIFEAQKIEWHE; SEQ ID NO:22; Beckett et al. 1999) and a hexahistidine tag (HHHHHH; SEQ ID NO:23), was cloned into a mammalian protein expression vector (pADD2) by In-Fusion (Takara Bio, Kusatsu, Shiga, Japan).

DNA encoding Sudan ebolavirus GP (Uganda/Gulu, 2000) ectodomain with the mucin-like domain deleted (residues 1-345, 506-656 of SEQ ID NO:4) or Bundibugyo ebolavirus GP (Uganda/Butalya, 2007) ectodomain with the mucin-like domain deleted (residues 1-312, 471-640 of SEQ ID NO:2) was cloned into pADD2 vector with a GCN4 or foldon trimerization domain, followed by an AviTag™ tag (GLNDIFEAQKIEWHE; SEQ ID NO:22) and a hexahistidine tag (HHHHHH; SEQ ID NO:23) on the C-terminus. GP-Fer was constructed by In-Fusion cloning of EBOV GP (residues 1-308 and 491-656 of SEQ ID NO:1) and H. pylori ferritin (Kanekiyo et al. 2013) (residues 5-168 of SEQ ID NO:7) with a Ser-Gly-Gly linker into the pADD2 vector. GP-Fer glycosylation variants were constructed by site-directed mutagenesis using GP-Fer as the backbone.

DNA fragments encoding the variable heavy chain (HC) and light chain (LC) were codon-optimized and synthesized by Integrated DNA Technologies (Coralville, Iowa). Fragments were inserted into an expression plasmid containing VRC01 HC and LC constant domains by In-Fusion.

All plasmids were sequence-confirmed by Sanger sequencing (Sequetech). For transfection purposes, the plasmids were transformed into Stellar cells (Takara Bio), isolated by Maxiprep kits (Macherey Nagel, Düren, Germany), filtered through a sterile 0.45 μm membrane in a biosafety cabinet, and stored at −20° C.

### D. Protein Expression and Purification.

All antigens and antibodies were expressed in Expi-293F cells. Expi-293F cells were cultured at 37° C. under constant shaking (120 rpm) in a humidified CO₂ (8%) incubator. Expi-293F cells were transfected at a density of 3-4×10⁶ cells/mL. For 200 mL transfection, the transfection mixture was made by adding 120 μg plasmid DNA into 20 mL expression media, followed by the dropwise addition of 260 μL FectoPro® transfection reagent (Polyplus, Illkirch-Graffenstaden, France) with vigorous mixing. For antibody production in 200 mL Expi-293F cells, the transfection mixtures contained 60 μg light-chain plasmid DNA and 60 μg heavy-chain plasmid DNA. The transfection mixtures were incubated at room temperature for 10 min before being transferred to Expi-293F cells. D-glucose (4 g/L, Sigma-Aldrich, Saint Louis, Missouri) and valproic acid (3 mM, Acros Organics, part of Thermo Fischer Scientific) were added to the cells immediately post-transfection to increase recombinant protein production. The cells were boosted again with D-glucose three days post-transfection and harvested on day four by centrifugation at 7000×g for five minutes. The supernatant was filtered through a 0.22-μm membrane for subsequent purification processes. Biotinylated GP was expressed using substantially the same protocol as above, but in the presence of BirA enzyme.

Histidine-tagged (“His-tagged”) proteins were purified with HisPur™ Ni-NTA resin (Thermo Fisher Scientific). Briefly, filtered supernatant from Expi-293F cells was mixed with Ni-NTA resin (1 mL resin per liter supernatant) and incubated at 4° C. overnight. The mixture was then passed through a gravity-flow column, washed with 20 mM imidazole in HEPES buffer saline (HBS, 20 mM HEPES, pH 7.4, 150 mM NaCl), and then eluted with 250 mM imidazole in HBS. Eluted fractions were concentrated with centrifugal filters (50 kDa MWCO, Millipore Sigma, Burlington, Massachusetts) and buffer-exchanged into HBS for size-exclusion chromatography using a Superose® 6 (Sigma-Aldrich) Increase 10/300 GL column (Cytiva). Peak fractions were pooled, concentrated, buffer-exchanged to HBS with 10% glycerol, and filtered through a 0.22 μm membrane.

GP-Fer and GP-Fer glycosylation variants were purified by anion-exchange chromatography, followed by size exclusion chromatography. Filtered supernatant from Expi-293F cells was directly applied to a HiTrap Q HP column (Cytiva) on ÄKTA Protein Purification System (Cytiva). The column was washed with Tris buffer (20 mM, pH 8.0), and ferritin nanoparticles were eluted with a NaCl gradient (0-1M). Nanoparticle-containing fractions were identified by Western blotting with mAb114, pooled, concentrated using centrifugal filters (100 kDa MWCO), and subsequently purified twice with size exclusion chromatography using SRT SEC-1000 column (Sepax Technologies, Newark, Delaware). Peak fractions were pooled, concentrated, buffer-exchanged to HBS with 10% glycerol, and filtered through a 0.22 μm membrane.

All the antibodies were purified with MabSelect PrismA™ (Cytiva) protein A chromatography. Filtered supernatant from Expi-293F cells was directly applied to a MabSelect PrismA™ column (Cytiva) on an ÄKTA Protein Purification System. The column was washed with HBS, and then the antibodies were eluted with glycine (100 mM, pH 2.8) into HEPES buffer (1 M, pH 7.4). The eluted antibody-containing fractions were concentrated and buffer-exchanged to HBS with 10% glycerol.

The concentration of all proteins was determined by measuring absorbance at 280 nm (A280), and protein purity was assessed by protein gel electrophoresis. Protein samples were flash-frozen in liquid nitrogen and stored at −20° C.

### E. Glycan Screening.

Based on the structure of EBOV GP (Protein Data Bank ID: 5JQ3), 16 sites on the surface of each GP protomer were selected for potential introduction of artificial glycosylation sites, where N-linked artificial glycosylation sites (N—X—S/T) were introduced by site-directed mutagenesis. The expression level of each GP-Fer glycosylation variant was then compared to expression of wild-type GP in Expi-293F cells via immunoblotting. Three days after transient transfection, Expi-293F cells were harvested via centrifugation at 7,000×g for 5 min. The supernatant samples were collected, mixed with Laemmli loading buffer (4×, Bio-Rad), boiled at 95° C. for 5 min, and then loaded onto protein gels (4-20% Mini-PROTEAN® TGX™ Precast Gels, Bio-Rad, Hercules, California). After gel electrophoresis, the proteins were transferred to 0.2 μm nitrocellulose membranes (Bio-Rad), and these blots were blocked with phosphate-buffered saline, 0.1% Tween-20 (PBST) with milk (10% nonfat dry milk, w/v, Bio-Rad) for one hour at room temperature. Primary antibody-mAb114 (2 mg/mL, 1:4000 dilution in PBST with milk) was then added to the blots for one-hour incubation, followed by addition of rabbit anti-human IgG, HRP conjugated (1:4000 dilution in PBST with milk) as the secondary antibody (45-minute incubation). The blots were rinsed in PBST for five minutes between steps, and developed using a Western blotting substrate (Pierce™ ECL, Thermo Fischer Scientific). The imaging was performed on a chemiluminescence imager (GE Amersham Imager 600), and the images were analyzed with Fiji image processing package ImageJ v2.3.0 software. GP-Fer glycosylation variants that retained >50% of GP expression were considered suitable for further analysis.

### F. Consurf Analysis.

Amino acid sequences of GP from five Ebolavirus species (Zaire-AAG40168.1, Sudan-AAU43887.1, Bundibugyo-AYI50307.1, Tai Forest-AAB37093.1, and Reston-AAC54891.1) were aligned using Clustal Omega (available from European Molecular Biology Laboratory European Bioinformatics Institute) to create a multiple sequence alignment (MSA). The MSA was analyzed by the Consurf server (Ashkenazy et al. 2016) based on the structure of Ebola GP (PDB ID: 5JQ3) and residues in the modified PDB file were re-colored in PyMOL software (Schrödinger Inc., New York, New York) using a Python script.

### G. Differential Scanning Fluorimetry.

Thermal melting profiles of proteins were measured by differential scanning fluorimetry on Prometheus NT.48 instrument (NanoTemper Technologies Inc., San Francisco, California). Protein samples (0.1 mg/mL) were loaded into glass capillaries (NanoTemper Technologies Inc.) and then subjected to a temperature gradient from 20 to 95° C. at a heating rate of 1° C. per min. Intrinsic fluorescence (350 nm and 330 nm) was recorded as a function of temperature. Thermal melting curves were plotted using the first derivative of the ratio (350 nm/330 nm). Melting temperatures were calculated automatically by the instrument (PR. ThermControl software) and represented the peaks in the thermal melting curves.

### H. Enzyme-Linked Immunosorbent Assays (ELISAs)

Nunc 96-well MaxiSorp™ plates (Thermo Fisher Scientific) were coated with streptavidin (4 μg/mL in DPBS, 60 μL per well, Thermo Fisher Scientific) for one hour at room temperature. The plates were washed three times with Milli-Q® (Millipore-Sigma, Burlington, Massachusetts) H₂O (300 μL) using a plate washer (ELx405 BioTek Instruments, Winooski, Vermont) and then blocked with ChonBlock (120 μL per well, Chondrex, Woodinville, Washington) overnight at 4° C. For subsequent steps, all the dilutions were made in DPBS with 0.05% Tween-20 and 0.1% BSA, and ELISA plates were rinsed with PBST (300 μL, three times) in between. Biotinylated Ebola GP proteins (wild-type, GP+2, GP+3, or GP+4 at 2 μg/mL) were added to the plates and incubated for one hour at room temperature. Then, serially diluted monoclonal antibodies (mAbs, starting from 200 nM, followed by 10-fold dilution) were added to the plates and incubated for another hour. Rabbit anti-human IgG, HRP-conjugated (1:4,000) was added for one-hour incubation before rinsing with PBST six times. ELISA plates were developed with the 3,3′,5,5′-Tetramethylbenzidine (TMB) substrate (1-Step Turbo-TMB, Thermo Fisher Scientific) for six minutes and terminated with sulfuric acid (2M). Absorbance at 450 nm was recorded on a microplate reader (Synergy™ HT, BioTek Instruments).

To test antibody binding to ferritin nanoparticles according to the present disclosure, they were hydrophobically coated onto Nunc 96-well MaxiSorp™ plates (2 μg/mL in DPBS, 60 μL per well) for one hour at room temperature. The plates were washed three times with Milli-Q® H₂O (300 μL) and then blocked with ChonBlock (120 μL per well) overnight at 4° C. Serially diluted mAbs were added to the plates, followed by secondary antibodies, TMB substrates, and sulfuric acid. Absorbance at 450 nm was recorded on a microplate reader (Synergy™ HT, BioTek Instruments).

### I. Size-Exclusion Chromatography-Multi-Angle Light Scattering (SEC-MALS) Analysis.

SEC-MALS analysis of ferritin nanoparticles was performed on 1260 Infinity II high-performance liquid chromatography system (Agilent Technologies, Santa Clara, California) coupled with a miniDAWN® and Optilab® detectors (Wyatt Technology Corporation, Santa Barbara, California) for light scattering and refractive index analysis. Purified GP-Fer, GP-Fer glycosylation variants, and wild-type ferritin (10 μg of each sample) were loaded onto an SRT SEC-1000 column (4.6×300 mM, Sepax Technologies) sequentially for analysis. ASTRA software (Wyatt Technology Corporation) was used for quantitative analysis of molar mass of the ferritin nanoparticles.

### J. Dynamic Light Scattering and Transmission Electron Microscopy.

Ferritin nanoparticles (GP-Fer, GP+2-Fer, GP+3-Fer, or GP+4-Fer) were diluted to 0.05 mg/mL with DPBS and filtered through 0.22 μm membrane before analysis. Hydrodynamic size of ferritin nanoparticles was then measured on Zetasizer Nano ZS instrument with 10 mW helium-neon laser and thermoelectric temperature controller (Malvern Panalytical Inc., Westborough, Massachusetts). Before each sample, the temperature of the instrument was equilibrated for one minute at 25° C.

Ferritin nanoparticles solutions (0.1 mg/mL, 10 μL) were pipetted onto a carbon-coated copper transmission electron microscopy (TEM) grid (Ted Pella, Redding, California) and incubated for five minutes, followed by negative staining with uranyl acetate (2%, w/v) for 1.5 min. TEM grids were dried overnight. TEM images were acquired on a Tecnai T12 cryo-electron microscope (FEI Company, Hillsboro, Oregon) operating with an acceleration voltage of 120 kV.

### K. Mouse Immunization Studies.

All animals were maintained in accordance with the Public Health Service Policy for “Human Care and Use of Laboratory Animals” under a protocol approved by the Stanford University Administrative Panel on Laboratory Animal Care (APLAC-33709). Female BALB/c mice (6-8 weeks) were purchased from Jackson Laboratory (Bar Harbor, Maine) and female C57BL/6 mice (6-8 weeks) were purchased from Jackson Laboratory or Charles River Laboratories (Wilmington, Massachusetts).

To compare immunogenicity of GP and GP-Fer nanoparticles, two groups of BALB/c mice (n=10) were immunized with 5 μg protein antigens adjuvanted with 10 μg Monophosphoryl lipid A (MPLA, Invivogen, San Diego, California) and 10 μg Quil-A® (Invivogen) via subcutaneous injection on days 0 and 21 (week 3). To compare immunogenicity of GP-Fer glycosylation variants, four groups of C57BL/6 mice were immunized with 5 μg protein antigens adjuvanted with 10 μg MPLA and 10 μg Quil-AR via subcutaneous injection on days 0, 21, and 42. To examine a different set of adjuvants, three groups of C57BL/6 mice (n=9) were immunized with 5 μg protein antigens adjuvanted with 500 μg alum (Alhydrogel®, Invivogen) and 20 μg CpG (ODN 1826, Invivogen) via subcutaneous injection on days 0, 21, and 42. To investigate a different immunization regimen, three groups of C57BL/6 mice (n=10) were immunized with 5 μg protein antigens adjuvanted with 10 μg MPLA and 10 μg Quil-A® via subcutaneous injection on days 0, 42, and 126. Pre-immune, interim and final blood samples were collected by retro-orbital bleeding into serum gel tubes (Sarstedt, Nümbrecht, Germany). Serum gel tubes were centrifuged at 10,000×g for 6 min, and the sera were collected and stored at −80° C.

### L. Serum ELISAs.

Nunc 96-well MaxiSorp™ plates (Thermo Fisher Scientific) were coated with streptavidin (4 μg/mL in DPBS, 60 μL per well) for one hour at room temperature. These plates were washed three times with MilliQ®-purified H₂O (300 μL) using a plate washer and then blocked with ChonBlock (120 μL per well) overnight at 4° C. For subsequent steps, all dilutions were made in DPBS with 0.05% Tween-20 and 0.1% BSA, and ELISA plates were rinsed with PBST (300 μL, three times) in between. Biotinylated Ebola GP (wild-type, 2 μg/mL) was added to the plates and incubated for one hour at room temperature. To test cross-reactive binding, biotinylated Sudan or Bundibugyo GP (2 μg/mL) was used instead of Ebola GP for this step. Mouse antisera were serially diluted (5-fold dilution) and then added to the ELISA plates for one-hour incubation at room temperature. Goat anti-mouse IgG, HRP-conjugated (1:4,000) was added for one-hour incubation before rinsing with PBST six times. ELISA plates were developed with the TMB substrate for six minutes and terminated with sulfuric acid (2M). Absorbance at 450 nm was recorded on a microplate reader (Synergy™ HT, BioTek).

### M. Pseudotyped Lentiviruses.

Ebola and Sudan GP-pseudotyped lentiviruses (EBOV, SUDV) encoding a luciferase-ZsGreen reporter were produced in HEK-293T cells by co-transfection of five plasmids (Crawford et al. 2020). This five-plasmid system included a packaging vector (pHAGE-Luc2-IRES-ZsGreen, a plasmid encoding full-length Ebola or Sudan GP (pCDNA3.1 EBOV FL-GP or pCDNA3.1 SUDV FL-GP), and three helper plasmids (pHDM-Hgpm2, pHDM-Tat1b, and pRC-CMV_Rev1b). One day before transfection, HEK-293T cells were seeded in 10-cm Petri dishes (5×10⁶ cells per Petri dish). Transfection mixture was prepared by adding five plasmids (10 μg packaging vector, 3.4 μg GP-encoding plasmid, and 2.2 μg of each helper plasmid) to 1 mL D10 medium, followed by the addition of BioT transfection reagent (30 μL, Bioland Scientific, Paramount, California) in a dropwise manner with vigorous mixing. After 10-minute incubation at room temperature, the transfection mixture was transferred to HEK-293T cells in a Petri dish. Culture medium was replenished 24 hours post-transfection. After another 48 hours, the viruses were harvested and filtered through a 0.45 μm membrane.

Bundibugyo GP-pseudotyped lentiviruses (BDBV) were produced using substantially the same method as above, but in Expi-293F cells. Expi-293F cells were adjusted to 3× 10⁶ cells per mL one day before transfection. For 200 mL Expi-293F cells, a transfection mixture was prepared by adding five plasmids (200 μg packaging vector, 68 μg plasmid encoding BDBV GP with the mucin domain deleted, and 44 μg of each helper plasmid) to 20 mL FreeStyle293/Expi-293 media, followed by addition of 600 μL BioT transfection reagent in a dropwise manner with vigorous mixing. After 10-minute incubation at room temperature, the transfection mixture was transferred to Expi-293F cells. Expi-293F cells were immediately boosted with D-glucose (4 g/L) and valproic acid (3 mM). After 72 hours, the viruses were harvested and filtered through a 0.45 μm membrane.

All pseudotyped lentiviruses (EBOV, SUDV, or BDBV) were aliquoted, flash-frozen in liquid nitrogen, stored at −80° C., and titrated in HEK-293T cells before further use.

N. Neutralization Assays with Monoclonal Antibodies.

Neutralization of three pseudotyped lentiviruses (EBOV, SUDV, or BDBV) was validated with monoclonal antibodies (mAbs) in HEK-293T cells. The cells were seeded in white-walled, clear-bottom 96-well plates at a density of 20,000 cells per well one day before the assay (day 0). On day 1, mAbs (2 μM in HBS with 10% glycerol) were filtered with 0.22 μm sterile membranes and diluted with D10 media. Subsequently, mAbs were serially diluted (10-fold dilution) in D10 media and mixed with lentiviruses (diluted in D10 medium, supplemented with polybrene, 1:1000, v/v) for one hour before being transferred to HEK-293T cells. On day 4, the medium was removed, and 100 μL of luciferase substrates (Britelite™ Plus, Perkin Elmer, Waltham, Massachusetts) were added to each well. Luminescent signals were recorded on a microplate reader (BioTek Synergy™ HT or Tecan M200 (Tecan Group Ltd. Männedorf, Switzerland). Percent infection was normalized to cells only (0% infection) and virus only (100% infection) on each plate. Neutralization assays were performed in biological replicates with technique duplicates.

### O. Serum Neutralization Assays.

Antisera were heat-inactivated (56° C., 30 min) before neutralization assays. Briefly, HEK-293T cells were seeded in white-walled, clear-bottom 96-well plates (20,000 cells per well) one day before the assay (day 0). On day 1, antisera were serially diluted in D10 media and then mixed with EBOV, SUDV, or BDBV (diluted in D10 medium, supplemented with polybrene, 1:1000, v/v) for one hour before being transferred to HEK-293T cells. On day 4, the medium was removed, and 100 μL of luciferase substrates (BriteLite™ Plus, Perkin Elmer) were added to each well. Luminescent signals were recorded on a microplate reader (BioTek Synergy™ HT or Tecan M200). Percent infection was normalized to cells only (0% infection) and virus only (100% infection) on each plate. Neutralization titers (NT₅₀) were calculated as the serum dilution where a 50% inhibition of infection was observed. Neutralization assays were performed in technical duplicates.

### P. Statistical Analyses.

The statistical analyses were performed using GraphPad Prism software (GraphPad Software, San Diego, California). Non-transformed data were presented as mean±standard deviation. Log-transformed data (ELISA titers and NT₅₀) were presented as geometric mean standard deviation. Comparisons of two groups were performed using the two-tailed Mann-Whitney U test. Comparisons of means of the treatment groups and the control group were performed using one-way ANOVA with a Dunnett's test. Comparisons of antigen-specific IgG or neutralization titers over time were performed using two-way ANOVA followed by a Bonferroni test. P values of 0.05 or less were considered significant and plotted.

### Example 2: Glycosylation Landscape on Ebola GP

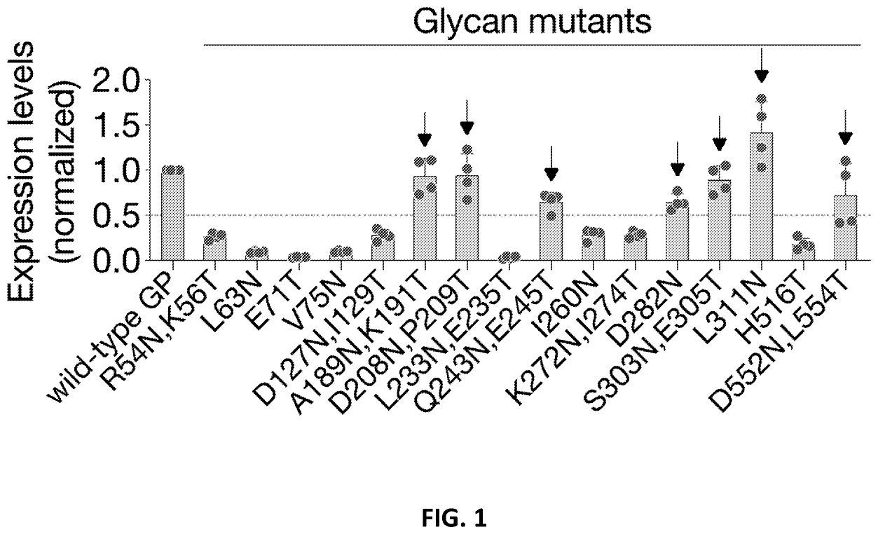

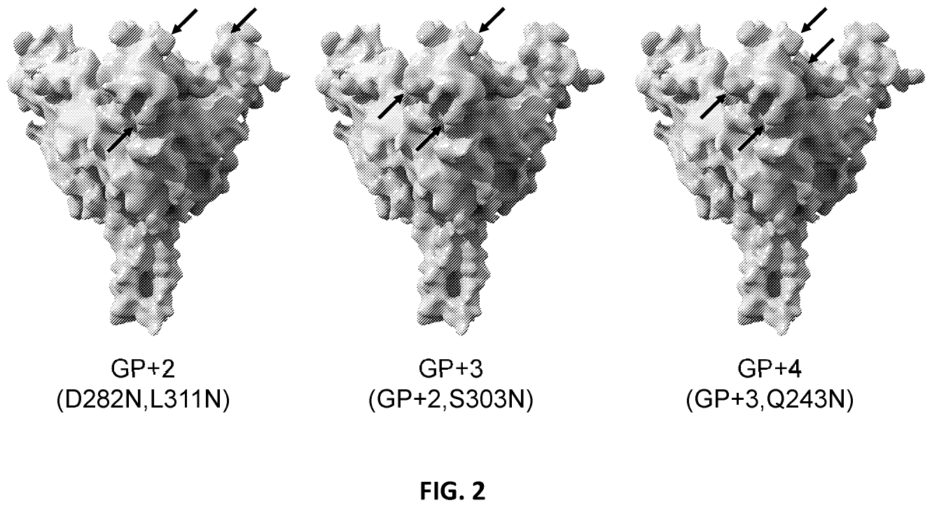

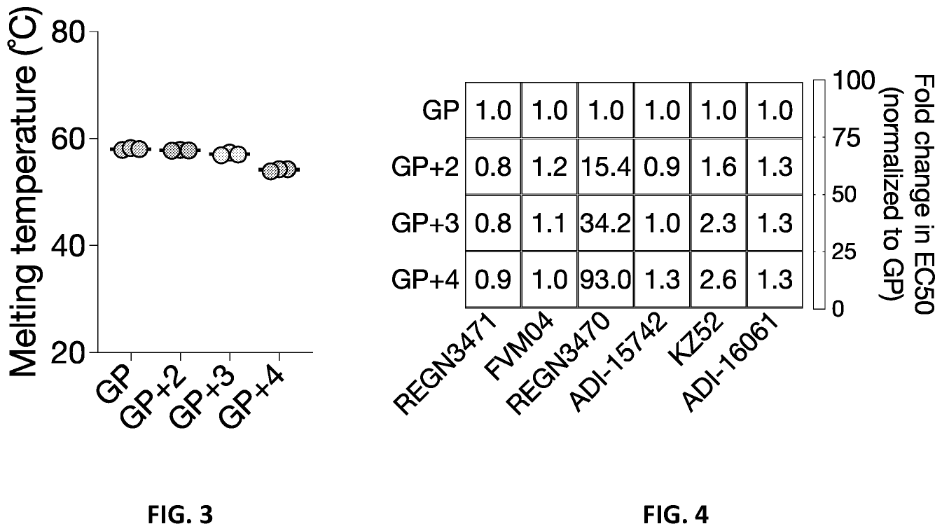

Based on the X-ray structure of EBOV GP (Zhao et al. 2016), 16 potential sites to install artificial N-linked glycosylation sites with Asn-X-Ser/Thr (N—X—S/T) motifs were selected in order to create single-glycan mutants via site-directed mutagenesis Since glycan installation may lead to protein misfolding or non-expression, the expression level of these glycan variants by Western blots were analyzed and compared with the expression levels of “wild-type GP” (EBOV GP polypeptide construct without artificial glycosylation site; SEQ ID NO:16) to identify the glycosylation variants suitable for further analysis. The results of the expression analysis are illustrated in FIG. 1. Whereas more than half of the mutations greatly reduced GP expression, seven mutants maintained >50% of GP expression compared to the expression of wild-type GP. In four of the above seven mutants, the newly introduced glycosylation sites were located around the glycan cap region, which is poorly conserved among different Ebolavirus species. In three other of the seven mutants, the glycosylation sites were located near the base of the GP trimer. It was decided to preferentially install glycosylation sites into the glycan cap to mask this poorly conserved region. By combining single-glycan mutants, two, three or four glycans were installed on each GP protomer, thus generating glycan GP variants GP+2-SEQ ID NO:17, GP+3-SEQ ID NO:18, and GP+4-SEQ ID NO:19 (the sequences are provided below, with artificial substitutions shown in bold and italics and artificial glycosylation sites shown in bold), as illustrated in FIG. 2, to afford different levels of epitope masking. The above three glycan GP variants were successfully expressed and purified to homogeneity. They exhibited a higher molecular weight than wild-type GP, when analyzed gel electrophoresis and Western blotting. GP, GP+2 and GP+3 also shared identical thermal melting profiles and melting temperature (Tₘ) around 58° C., while Tₘ of GP+4 shifted to a slightly lower temperature of 54° C., as illustrated in FIG. 3. This difference suggested that installation of four glycans may have slightly destabilized the protein structure, although all proteins were thermostable at the physiological temperature. To examine the degree of epitope-masking, the binding of six GP-specific mAbs to hyperglycosylated GP was measured by enzyme-linked immunosorbent assays (ELISAs). The results are summarized in FIG. 4. The mAbs used in ELISA recognize different epitopes on GP (REGN3471 (Pascal et al. 2014), FVM04 (Howell et al. 2016), REGN3470 (Pascal et al. 2014), ADI-15742 (Wec et al. 2017), KZ52 (Lee et al. 2008), ADI-16061 (Wec et al. 2017) and allow for rapid epitope-mapping. No noticeable difference was observed in mAb-binding to GP and of GP+2, GP+3, and GP+4 glycosylation variants, except for in the binding of REGN3470 targeting the glycan cap. As the number of glycans increased, the binding affinity of REGN3470 toward GP glycosylation variants gradually decreased, approaching 93-fold for GP+4. Taken together, the above results showed that addition of glycans to GP glycan cap created GP glycosylation variants with different levels of epitope masking.

TABLE 5
Amino Acid Sequences of EBOV GP and its Glycosylation Variants.

Amino acid sequence of ebolavirus GP protein - SEQ ID NO: 16
MGVTGILQLPRDRFKRTSFFLWVIILFQRTFSIPLGVIHNSTLQVSDVDKLVCRDKL
SSTNQLRSVGLNLEGNGVATDVPSATKRWGFRSGVPPKVVNYEAGEWAENCYNL
EIKKPDGSECLPAAPDGIRGFPRCRYVHKVSGTGPCAGDFAFHKEGAFFLYDRLAS
TVIYRGTTFAEGVVAFLILPQAKKDFFSSHPLREPVNATEDPSSGYYSTTIRYQATG
FGTNETEYLFEVDNLTYVQLESRFTPQFLLQLNETIYTSGKRSNTTGKLIWKVNPEI
DTTIGEWAFWETKKNLTRKIRSEELSFAGLITGGRRTRREAIVNAQPKCNPNLHYW
TTQDEGAAIGLAWIPYFGPAAEGIYIEGLMHNQDGLICGLRQLANETTQALQLFLR
ATTELRTFSILNRKAIDFLLQRWGGTCHILGPDCCIEPHDWTKNITDKIDQIIHDFVD
KTLPDQGDNDNWWTGWRQWIPAGIMKQIEDKIEEILSKIYHIENEIARIKKLIGEVA
SSSGLNDIFEAQKIEWHEAHHHHHHG
Amino acid sequence of ebolavirus GP + 2 protein - SEQ ID NO: 17 (artificial
substitutions D282N and L311N are shown in bold and italics; artificial glycosylation
sites are shown in bold)
MGVTGILQLPRDRFKRTSFFLWVIILFQRTFSIPLGVIHNSTLQVSDVDKLVCRDKL
SSTNQLRSVGLNLEGNGVATDVPSATKRWGFRSGVPPKVVNYEAGEWAENCYNL
EIKKPDGSECLPAAPDGIRGFPRCRYVHKVSGTGPCAGDFAFHKEGAFFLYDRLAS
TVIYRGTTFAEGVVAFLILPQAKKDFFSSHPLREPVNATEDPSSGYYSTTIRYQATG
FGTNETEYLFEVDNLTYVQLESRFTPQFLLQLNETIYTSGKRSNTTGKLIWKVNPEI
NTTIGEWAFWETKKNLTRKIRSEELSFAGNITGGRRTRREAIVNAQPKCNPNLHY
WTTQDEGAAIGLAWIPYFGPAAEGIYIEGLMHNQDGLICGLRQLANETTQALQLFL
RATTELRTFSILNRKAIDFLLQRWGGTCHILGPDCCIEPHDWTKNITDKIDQIIHDFV
DKTLPDQGDNDNWWTGWRQWIPAGIMKQIEDKIEEILSKIYHIENEIARIKKLIGEV
ASSSGLNDIFEAQKIEWHEAHHHHHHG
Amino acid sequence of ebolavirus GP + 3 protein included in a fusion protein -
SEQ ID NO: 18 (artificial substitutions D282N, L311N, S303N, and E305T are shown
in bold and italics; artificial glycosylation sites are shown in bold)
MGVTGILQLPRDRFKRTSFFLWVIILFQRTFSIPLGVIHNSTLQVSDVDKLVCRDKL
SSTNQLRSVGLNLEGNGVATDVPSATKRWGFRSGVPPKVVNYEAGEWAENCYNL
EIKKPDGSECLPAAPDGIRGFPRCRYVHKVSGTGPCAGDFAFHKEGAFFLYDRLAS
TVIYRGTTFAEGVVAFLILPQAKKDFFSSHPLREPVNATEDPSSGYYSTTIRYQATG
FGTNETEYLFEVDNLTYVQLESRFTPQFLLQLNETIYTSGKRSNTTGKLIWKVNPEI
NTTIGEWAFWETKKNLTRKIRNETLSFAGNITGGRRTRREAIVNAQPKCNPNLHY
WTTQDEGAAIGLAWIPYFGPAAEGIYIEGLMHNQDGLICGLRQLANETTQALQLFL
RATTELRTFSILNRKAIDFLLQRWGGTCHILGPDCCIEPHDWTKNITDKIDQIIHDFV
DKTLPDQGDNDNWWTGWRQWIPAGIMKQIEDKIEEILSKIYHIENEIARIKKLIGEV
ASSSGLNDIFEAQKIEWHEAHHHHHHG
Amino acid sequence of ebolavirus GP + 4 protein included in a fusion protein -
SEQ ID NO: 19 (artificial substitutions D282N, L311N, S303N, E305T, Q243N, and
E245Tare shown in bold and italics; artificial glycosylation sites are shown in bold)
MGVTGILQLPRDRFKRTSFFLWVIILFQRTFSIPLGVIHNSTLQVSDVDKLVCRDKL
SSTNQLRSVGLNLEGNGVATDVPSATKRWGFRSGVPPKVVNYEAGEWAENCYNL
EIKKPDGSECLPAAPDGIRGFPRCRYVHKVSGTGPCAGDFAFHKEGAFFLYDRLAS
TVIYRGTTFAEGVVAFLILPQAKKDFFSSHPLREPVNATEDPSSGYYSTTIRYQATG
FGTNETEYLFEVDNLTYVNLTSRFTPQFLLQLNETIYTSGKRSNTTGKLIWKVNPEI
NTTIGEWAFWETKKNLTRKIRNETLSFAGNITGGRRTRREAIVNAQPKCNPNLHY
WTTQDEGAAIGLAWIPYFGPAAEGIYIEGLMHNQDGLICGLRQLANETTQALQLFL
RATTELRTFSILNRKAIDFLLQRWGGTCHILGPDCCIEPHDWTKNITDKIDQIIHDFV
DKTLPDQGDNDNWWTGWRQWIPAGIMKQIEDKIEEILSKIYHIENEIARIKKLIGEV
ASSSGLNDIFEAQKIEWHEAHHHHHHG

### Example 3: Display of GP and its Glycosylation Variants on Ferritin Nanoparticles

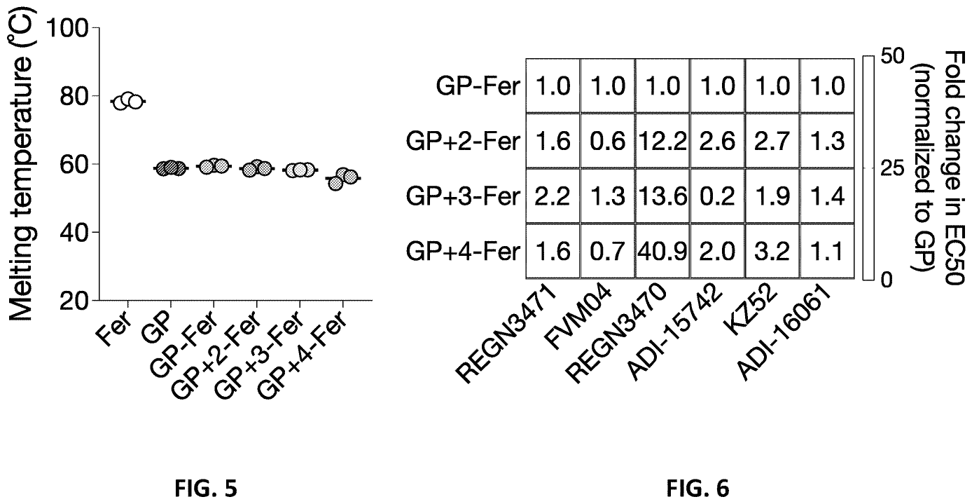

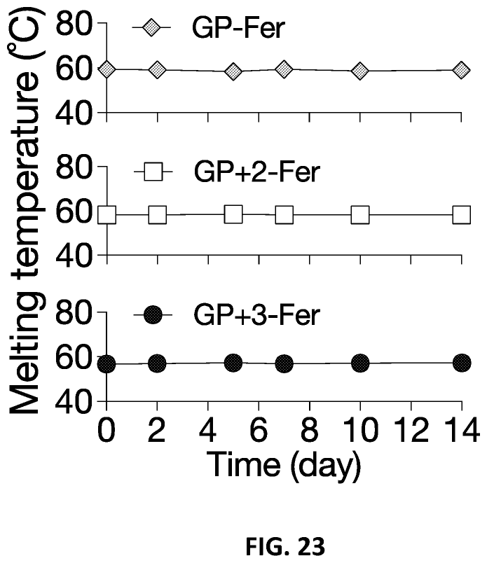

Although protein-based subunit vaccines are often less immunogenic than their viral vector counterparts, multivalent presentation of antigens on nanoparticles may enhance antigen valency and immunogenicity (Kato et al. 2020). Ferritin nanoparticles were generated from fusion proteins incorporating wild-type GP amino acid sequences (GP-Fer) and amino acid sequences of GP glycosylation variants discussed in the previous example (GP+2-Fer-SEQ ID NO:13, GP+3-Fer-SEQ ID NO: 14, and GP+4-Fer-SEQ ID NO:15). Ferritin nanoparticles presented eight copies of the GP trimer on the surface at its threefold axes of symmetry (Cho et al. 2009). GP-Fer, GP+2-Fer, GP+3-Fer, and GP+4-Fer were expressed and purified to homogeneity. GP+2-Fer, GP+3-Fer, and GP+4-Fer exhibited higher molecular weight than GP-Fer when analyzed by gel electrophoresis and Western blotting. The thermal melting profiles or Tₘ of GP+2-Fer, GP+3-Fer, and GP+4-Fer did not substantially change, as compared to GP-Fer, since Fer only unfolded at a high temperature (~80° C.). Tₘ of GP+4-Fer was slightly lower than that of GP-Fer, GP+2-Fer or GP+3-Fer, as illustrated in FIG. 5. Thermal stability of GP-Fer, GP+2-Fer and GP+3-Fer after incubation at 37° C. for 2, 5, 7, 10, or 14 days is also illustrated in FIG. 23, confirming thermal stability of these nanoparticles over time. REGN3470 was the only mAb that gradually lost its GP-binding capacity as the number of glycans increased, as illustrated in FIG. 6, while the other five mAbs showed similar binding profiles for GP-Fer, GP+2-Fer, GP+3-Fer, and GP+4-Fer.

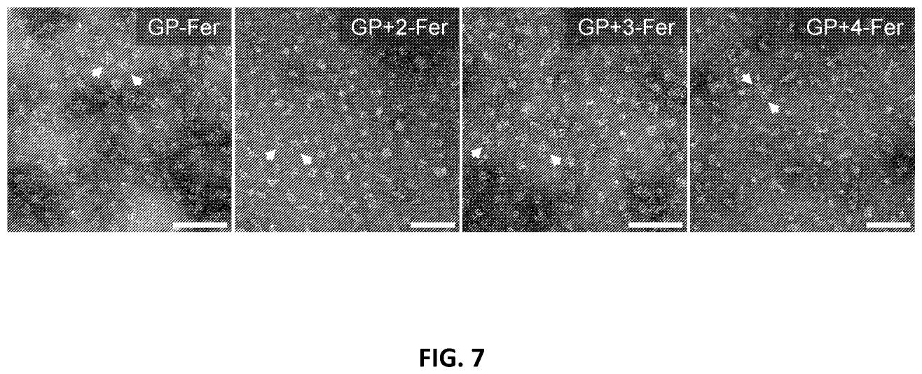

The homogeneity of GP-Fer, GP+2-Fer, GP+3-Fer, and GP+4-Fer was examined with multi-angle light scattering coupled to size exclusion chromatography (SEC-MALS) using wild-type Fer as a control. All samples eluted as single peaks indicative of monodisperse nanoparticles. The installation of glycans gradually increased the Mw of GP-Fer from 1.7 to 2.0 megadalton (MDa). Consistent with MALS, dynamic light scattering confirmed the monodispersity of these ferritin nanoparticles with a hydrodynamic diameter of 22 nm. Ferritin nanoparticles were visualized with TEM, as illustrated in FIG. 7.

### Example 4: Immunogenicity of Ferritin Nanoparticles

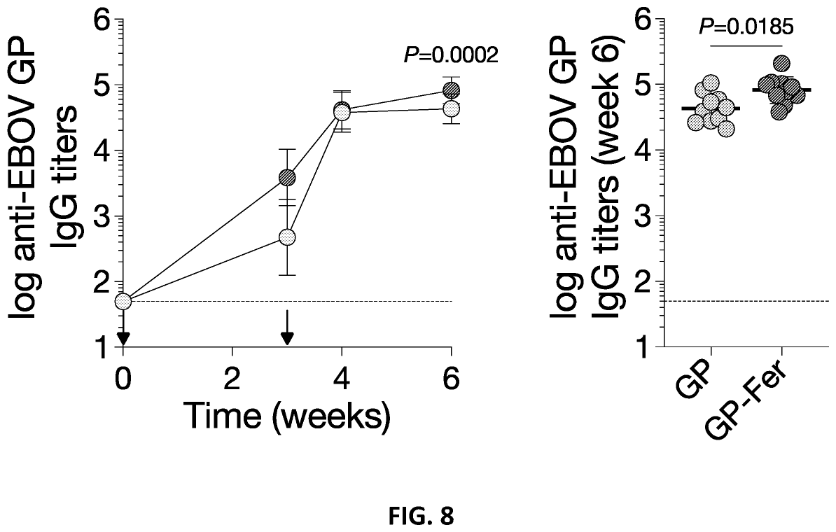

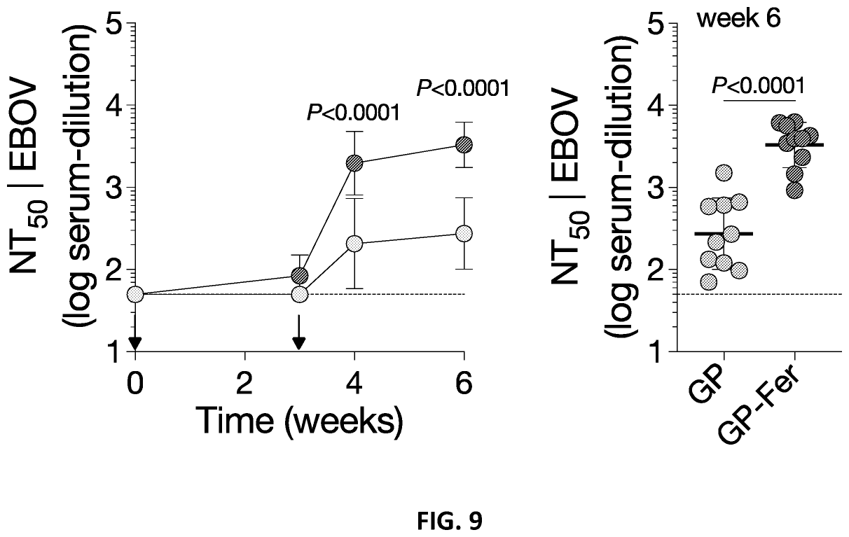

To investigate whether multimerization on ferritin increased the immunogenicity of GP, two groups of mice were immunized with GP-Fer ferritin nanoparticles or GP, each adjuvanted with MPLA (Okemoto et al. 2006) and Quil-A® (Reed et al. 2013), as described in Example 1. A prime and boost regimen with GP-Fer ferritin nanoparticles elicited a rapid immune response in mice, as well as significantly higher GP-specific IgG titers than GP did three weeks post-boost, as illustrated in FIG. 8. To determine whether increased antibody titers correlated with better neutralizing activity, EBOV GP-pseudotyped lentiviruses were generated and tested in neutralization assays with mouse antisera. Although both GP-Fer ferritin nanoparticles and GP induced neutralizing antisera against EBOV after the boost, average neutralization titers (NT₅₀) in the GP-Fer group of mice were tenfold higher than in the GP group, as illustrated in FIG. 9.

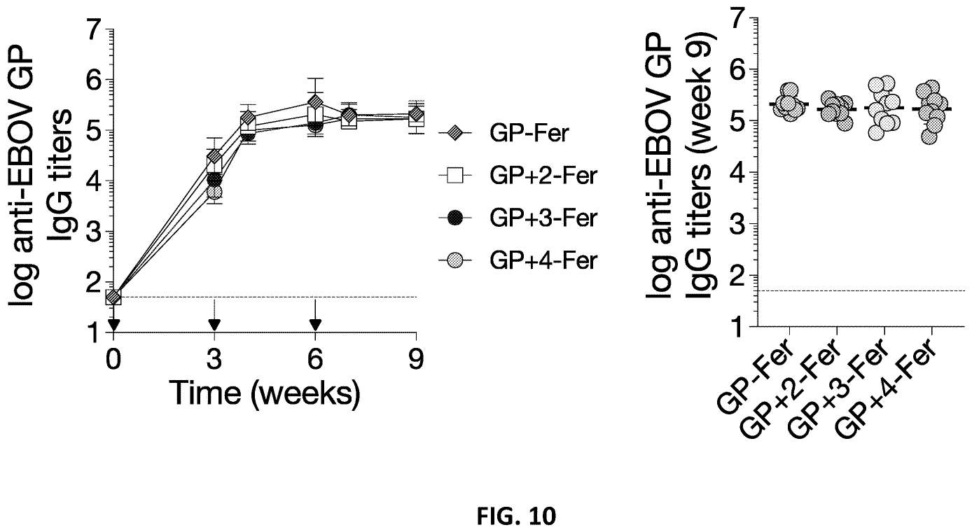

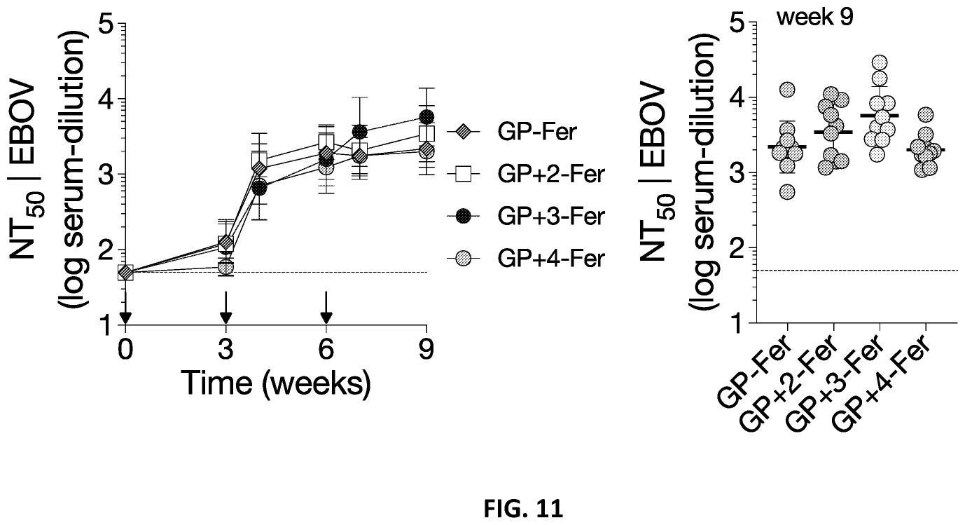

In subsequent experiments, four groups of mice were immunized with GP-Fer, GP+2-Fer, GP+3-Fer, and GP+4-Fer ferritin nanoparticles, each of which was adjuvanted with MPLA/Quil®-A to investigate whether GP-Fer glycosylation variants prompted a cross-reactive response. Three weeks after the prime, mice from all groups developed EBOV GP-specific IgG responses, while titers in GP+3-Fer and GP+4-Fer groups were significantly lower than those in the GP-Fer group, indicating reduced immunogenicity resulted from increased glycosylation. To determine whether the installed glycans shielded antibody responses away from the glycan cap, competition ELISAs were performed, in which ELISA plates coated with GP-Fer, GP+2-Fer, GP+3-Fer, and GP+4-Fer ferritin nanoparticles were pre-incubated with a saturating concentration of REGN3470 before adding antisera. While competition with REGN3470 led to a twofold decrease in binding titers of the GP-Fer group, less competition as the number of glycans increased. The difference in antibody responses also correlated with NT₅₀, where GP+4-Fer induced the lowest neutralizing activity among all the antigens. Nonetheless, two booster injections of each antigen elevated antibody responses to high titers with potent neutralizing activity against EBOV in all groups, as illustrated in FIG. 10 and FIG. 11.

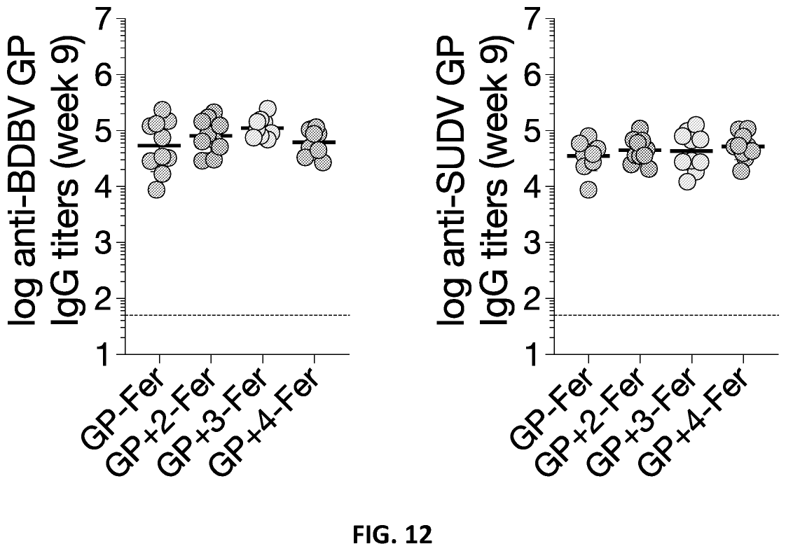

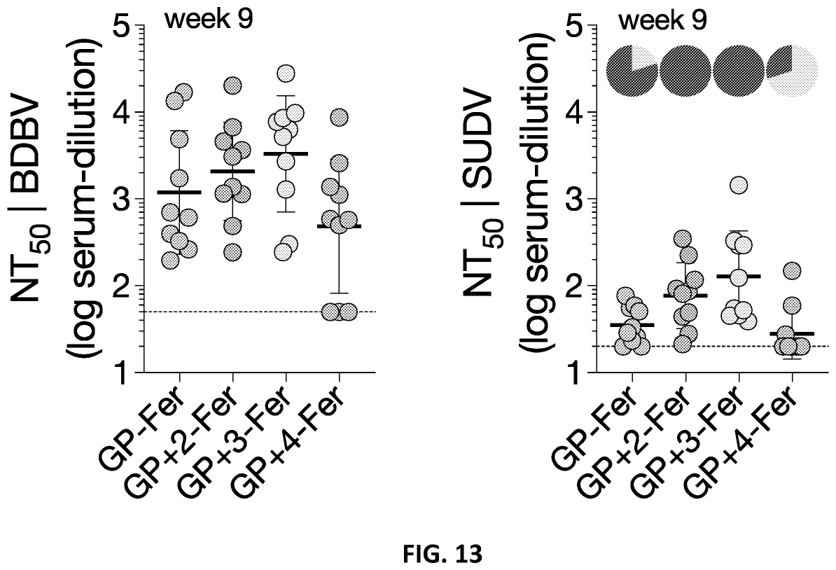

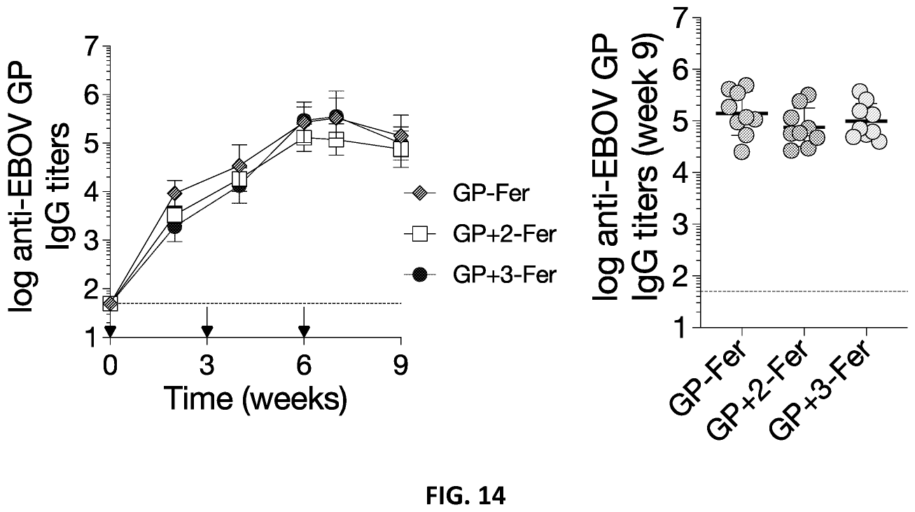

Cross-reactivity of the endpoint antisera from the immunized mice was examined. All the antigens induced cross-reactive antibodies to BDBV GP and SUDV GP with titers about tenfold lower than those against EBOV GP, as illustrated in FIG. 12. It was also found that cross-reactive antisera competed with FMV04, a known cross-neutralizing mAb targeting the head of GP in binding to SUDV GP, while less competition was observed with ADI-15742, another cross-neutralizing mAb targeting the fusion loop of GP. Using BDBV GP-or SUDV GP-pseudotyped lentiviruses, it was examined whether cross-reactive binding translated to cross-neutralization. The antisera from all but three mice in GP+4-Fer group cross-neutralized BDBV GP-pseudotyped lentivirus, as illustrated in FIG. 13. The average NT₅₀ in GP+2-Fer and GP+3-Fer groups were higher than those in GP-Fer and GP+4-Fer groups. A similar trend was observed in the cross-neutralization of SUDV GP-pseudotyped lentivirus, with GP+2-Fer and GP+3-Fer ferritin nanoparticles outperforming GP-Fer and GP+4-Fer ferritin nanoparticles, although neutralization titers were generally lower. These results suggested that focusing antibody responses away from the variable glycan cap and toward conserved epitopes afforded cross-neutralizing activity.

### Example 5: Investigation of Different Immunization Regimens

Although, as discussed above, installation of four glycans afforded the best epitope masking of the glycan cap in monoclonal antibody binding analysis, GP+4-Fer ferritin nanoparticles induced weaker cross-neutralizing activity than GP+2-Fer or GP+3-Fer ferritin nanoparticles. This result suggested that installation of the fourth glycan did not further improve the production of cross-reactive antibodies. In view of this, different immunization regimens using GP+2-Fer and GP+3-Fer ferritin nanoparticles, in comparison with GP-Fer ferritin nanoparticles, were investigated.

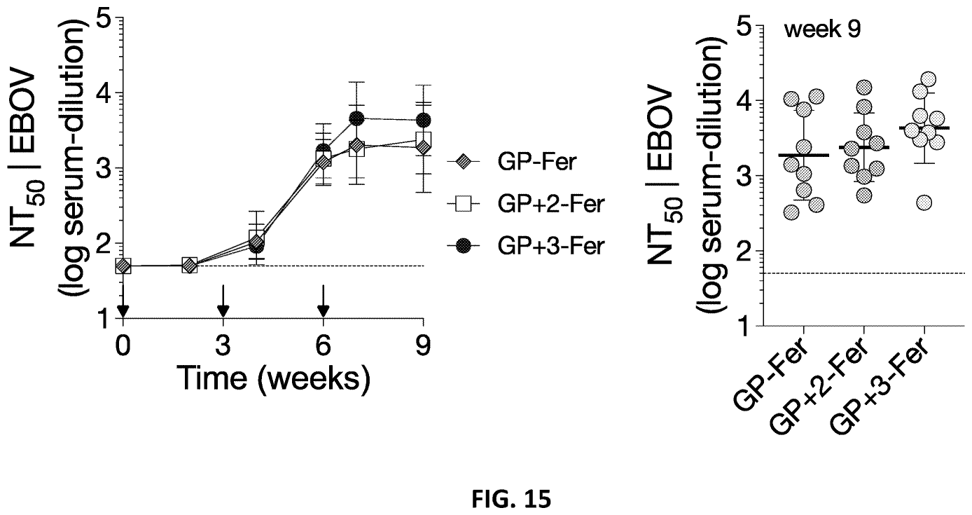

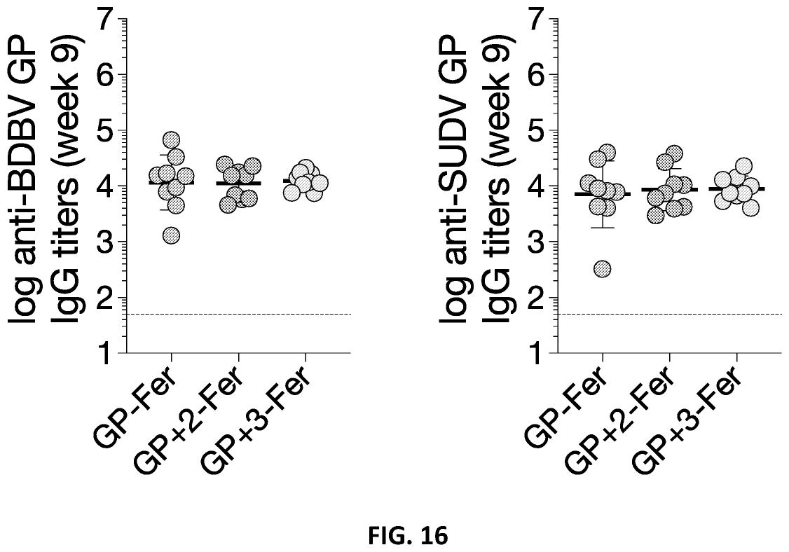

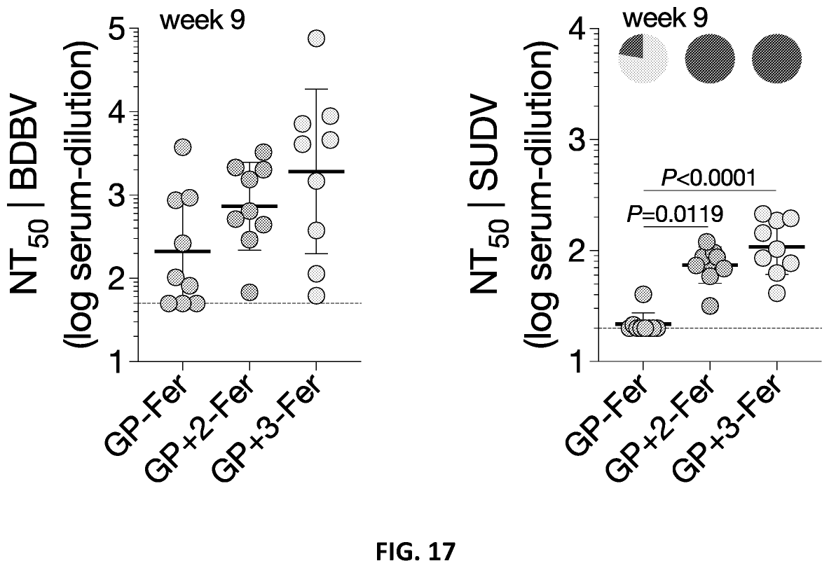

Three groups of mice were with GP-Fer, GP+2-Fer or GP+3-Fer ferritin nanoparticles, adjuvanted with aluminum hydroxide (alum) and CpG oligodeoxynucleotides. CpG is a Toll-like receptor 9 agonist that potently enhances the host immune response to antigens (Krieg et al. 1995). GP-Fer ferritin nanoparticles elicited higher EBOV GP-specific IgG titers than GP+2-Fer or GP+3-Fer ferritin nanoparticles at early time points, while mice from all groups developed similar responses after two boosts, as illustrated in FIG. 13. Unlike in the previous immunization using MPLA/Quil®-A as an adjuvant (discussed above), in which mouse antisera neutralized EBOV one week after the first boost, when alum/CpG was used as an adjuvant, development of strong neutralizing activity occurred three weeks after the first boost, as illustrated in FIG. 15. Nonetheless, antisera from all mice potently neutralized EBOV after two booster injections. Despite cross-binding of BDBV GP and SUDV GP, as illustrated in FIG. 16, antisera from only several mice in GP-Fer group cross-neutralized BDBV GP-pseudotyped lentivirus or SUDV GP-pseudotyped lentivirus, as illustrated in FIG. 17. In contrast, all the mice in GP+2-Fer and GP+3-Fer groups showed cross-neutralizing activity against BDBV GP-pseudotyped lentivirus and SUDV GP-pseudotyped lentivirus.

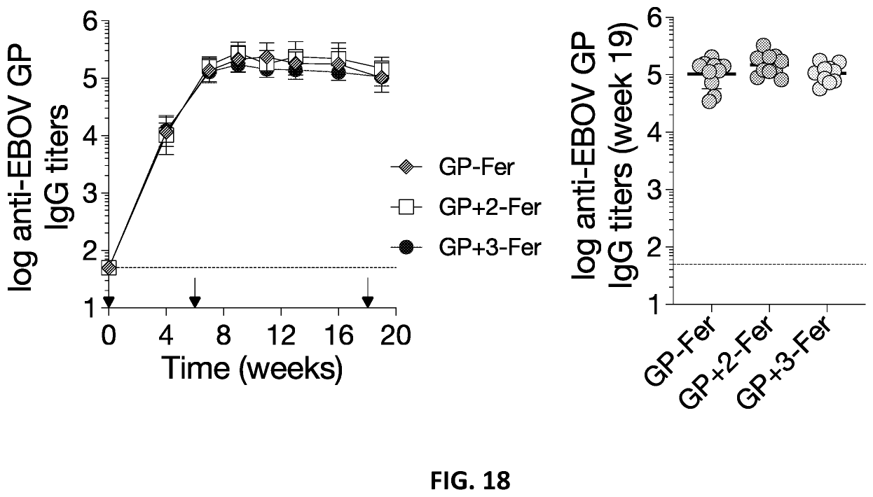

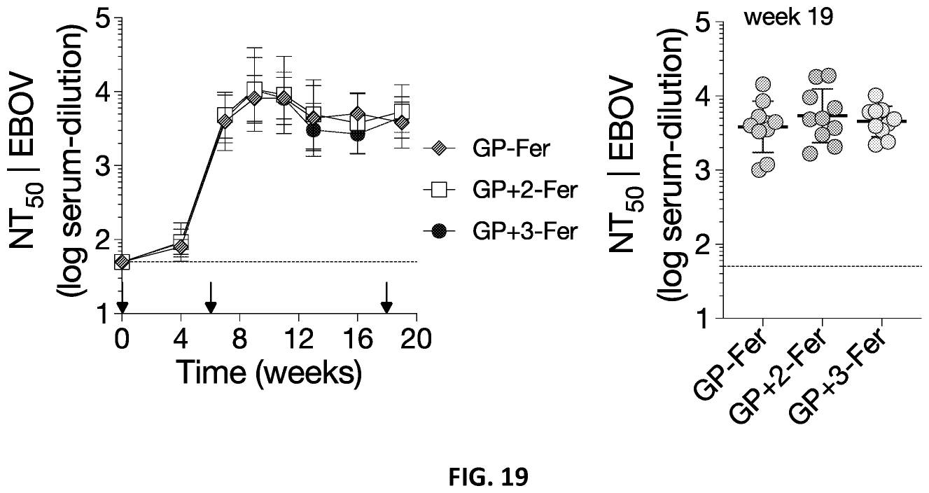

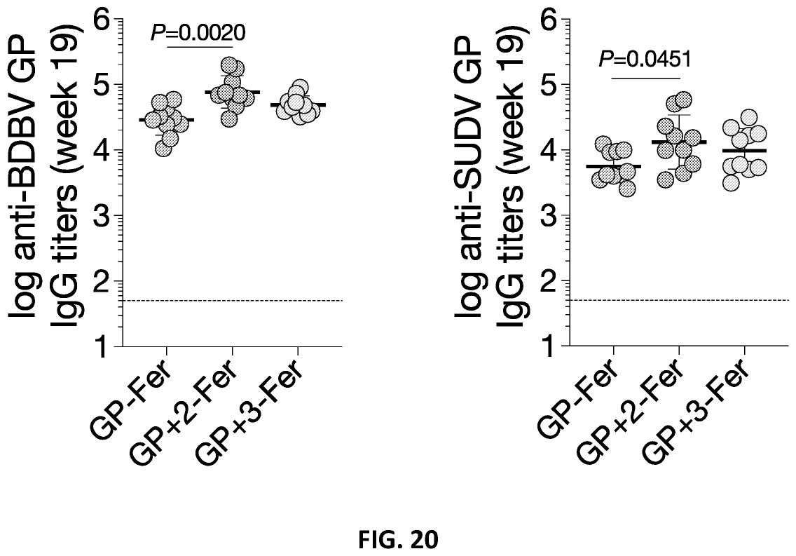

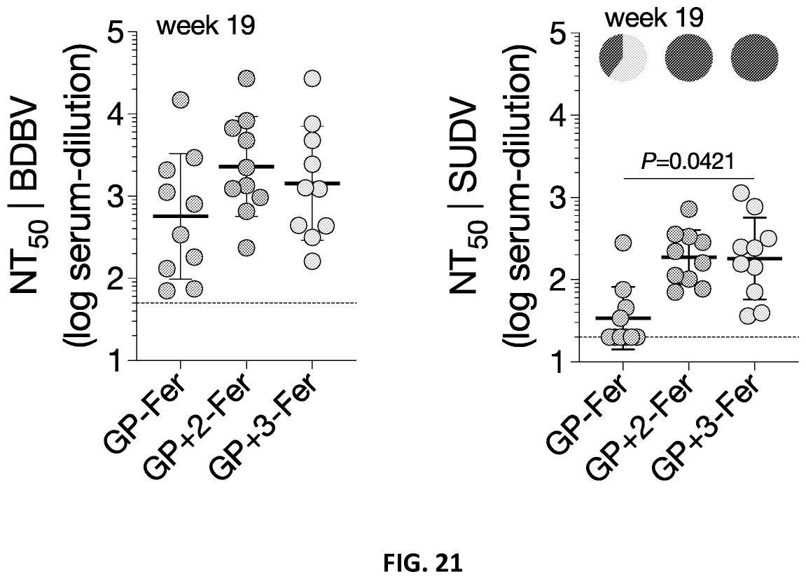

An immunization regimen with delayed boost was investigated, with three injections within six weeks. Three groups of mice were immunized with GP-Fer, GP+2-Fer or GP+3-Fer ferritin nanoparticles adjuvanted with MPLA/Quil®-A on week 0, 6 and 18. Despite exhibiting similar titers against EBOV GP at week four, GP+2-Fer and GP+3-Fer groups induced significantly higher cross-binding titers against BDBV GP and SUDV GP. The second injection at week six elevated EBOV GP-specific IgG titers (as illustrated in FIG. 18) and substantially increased the neutralizing response against EBOV in all groups (as illustrated in FIG. 19). Delayed boost regimen also induced cross-neutralizing activity against BDBV and SUDV, although GP-Fer ferritin nanoparticles had the least effect. Antibody titers and NT₅₀ remained at high levels with negligible differences among groups for more than ten weeks before a second boost was administered. Similar to earlier discussed results, mice from all groups showed cross-binding titers against BDBV GP and SUDV GP at one week post-2nd boost, as illustrated in FIG. 20. Despite a minimal effect on NT₅₀ against EBOV, all the mice developed cross-neutralizing activity against BDBV with the lowest titers in the GP-Fer group, as illustrated in FIG. 21. In addition, the antisera from all the mice in the GP+2-Fer and GP+3-Fer groups cross-neutralized SUDV, whereas the antisera from only four of ten mice in the GP-Fer group did. These above immunization studies showed that addition of more than one glycan on GP-Fer ferritin nanoparticles improved their cross-neutralizing activity.

### Example 6: Live Authentic Ebola Virus Assay

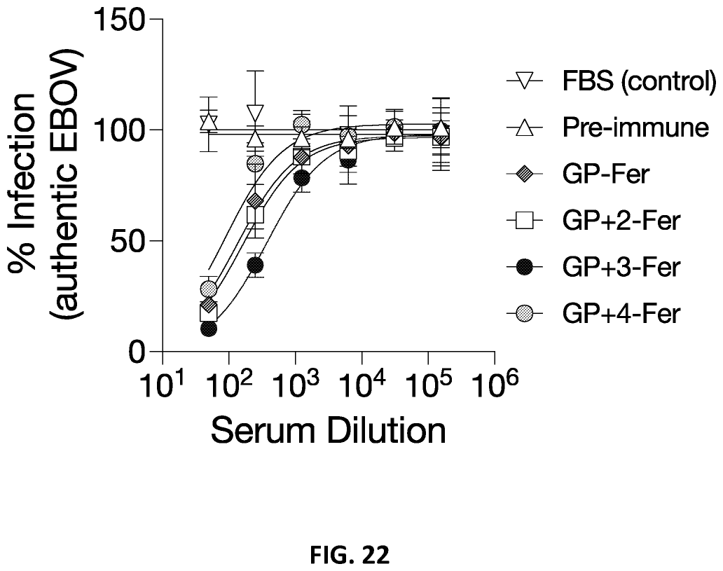

Live authentic Ebola virus assay was conducted as follows. Vero E6 cells were seeded in 96-well plates at 75-90% confluency and taken into the biosafety level 4 (BSL-4) facility after overnight incubation. Pooled antisera generated from each antigen (GP-Fer, GP+2-Fer, GP+3-Fer, and GP+4-Fer) were serially diluted before incubation with authentic EBOV (strain Zaire Mayinga, MOI=0.4) for one hour. The mixture was then transferred to cells and incubated for another 48 hours in a humidified CO2 (5%) incubator at 37° C. After incubation, the plates were decanted into 5% Microchem® (Round Rock, Texas) disinfectant and then submerged into 10% neutral buffered formalin for inactivation at 4° C. overnight. The plates were then removed from BSL-4 and stained with a mouse anti-GP antibody (1:1500, IBT 0201-020) with Hoechst 33342 as the counterstain, followed by high content imaging for analysis of infectivity using CellProfiler open-source software. Percent infection was normalized to cells only (0% infection) and virus only (100% infection) on each plate. The results are illustrated in FIG. 22 and validate live Ebola virus neutralization potency of the antisera elicited by the tested antigens.

### PUBLICATIONS CITED IN THIS DISCLOSURE

1. Altschul et al. Basic local alignment search tool. J. Mol. Biol. 215:403-410. (1990)
2. Altschul et al. Gapped BLAST and PSI-BLAST: a new generation of protein database search programs. Nucleic Acids Res. 25:3389-3402 (1977)
3. Ashkenazy et al. ConSurf 2016: an improved methodology to estimate and visualize evolutionary conservation in macromolecules. Nucleic Acids Res. 44, W344-50 (2016).
4. Beckett et al. A minimal peptide substrate in biotin holoenzyme synthetase-catalyzed biotinylation. Protein Sci. 8, 921-929 (1999).
5. Bengtsson et al. Matrix-M adjuvant enhances antibody, cellular and protective immune responses of a Zaire Ebola/Makona virus glycoprotein (GP) nanoparticle vaccine in mice. Vaccine 34, 1927-1935 (2016).
6. Brecher et al. Cathepsin cleavage potentiates the Ebola virus glycoprotein to undergo a subsequent fusion-relevant conformational change. J. Virol. 86, 364-372 (2012).
7. Brouwer et al. Two-component spike nanoparticle vaccine protects macaques from SARS-COV-2 infection. Cell 184, 1188-1200.e19 (2021).
8. Chandran et al. Endosomal proteolysis of the Ebola virus glycoprotein is necessary for infection. Science 308, 1643-1645 (2005).
9. Chen et al. Fusion protein linkers: Property, design and functionality. Adv. Drug Deliv. Rev. 65 (10): 1357-1369 (2013) (doi: 10.1016/j.addr.2012.9.039).
10. Cho et al. The crystal structure of ferritin from Helicobacter pylori reveals unusual conformational changes for iron uptake. J. Mol. Biol. 390, 83-98 (2009).
11. Corti et al. Protective monotherapy against lethal Ebola virus infection by a potently neutralizing antibody. Science 351, 1339-1342 (2016).
12. Côté et al. Small molecule inhibitors reveal Niemann-Pick Cl is essential for Ebola virus infection. Nature 477, 344-348 (2011).
13. Crawford et al. Protocol and Reagents for Pseudotyping Lentiviral Particles with SARS-CoV-2 Spike Protein for Neutralization Assays. Viruses 12, (2020).
14. Duan et al. Glycan Masking Focuses Immune Responses to the HIV-1 CD4-Binding Site and Enhances Elicitation of VRC01-Class Precursor Antibodies. Immunity 49, 301-311.e5 (2018).
15. Eggink et al. Guiding the immune response against influenza virus hemagglutinin toward the conserved stalk domain by hyperglycosylation of the globular head domain. J. Virol. 88, 699-704 (2014).
16. European Medicines Agency. Zabdeno, INN-Ebola vaccine (Ad26.ZEBOV-GP [recombinant]). https://www.ema.europa.eu/en/documents/product-information/zabdeno-epar-product-information_en.pdf.
17. Fan et al. Multilamellar Vaccine Particle Elicits Potent Immune Activation with Protein Antigens and Protects Mice against Ebola Virus Infection. ACS Nano 13, 11087-11096 (2019).
18. “FDA Approves First Treatment for Ebola Virus.” FDA News Release. Available at fda.gov/news-events/press-announcements/fda-approves-first-treatment-ebola-virus. Oct. 14, 2020.
19. “FDA Approves Treatment for Ebola Virus.” FDA News Release. Available at fda.gov/drugs/drug-safety-and-availability/fda-approves-treatment-ebola-virus. Dec. 21, 2020.
20. “First FDA-approved vaccine for the prevention of Ebola virus disease, marking a critical milestone in public health preparedness and response.” FDA News Release. Available at fda.gov/news-events/press-announcements/first-fda-approved-vaccine-prevention-ebola-virus-disease-marking-critical-milestone-public-health. Dec. 19, 2019.
21. Feldmann et al. Ebola. N. Engl. J. Med. 382, 1832-1842 (2020).
22. Feldmann et al. Ebola: lessons on vaccine development. Annu. Rev. Microbiol. 72, 423-446 (2018).
23. Flyak et al. Broadly neutralizing antibodies from human survivors target a conserved site in the Ebola virus glycoprotein HR2-MPER region. Nat. Microbiol. 3, 670-677 (2018).
24. Flyak et al. Cross-Reactive and Potent Neutralizing Antibody Responses in Human Survivors of Natural Ebolavirus Infection. Cell 164, 392-405 (2016).
25. He et al. Presenting native-like trimeric HIV-1 antigens with self-assembling nanoparticles. Nat. Commun. 7, 12041 (2016).
26. He et al. Single-component multilayered self-assembling nanoparticles presenting rationally designed glycoprotein trimers as Ebola virus vaccines. Nat. Commun. 12, 2633 (2021).
27. Henao-Restrepo et al. Efficacy and effectiveness of an rVSV-vectored vaccine in preventing Ebola virus disease: final results from the Guinea ring vaccination, open-label, cluster-randomised trial (Ebola ça Suffit!). Lancet 389, 505-518 (2017).
28. Henikoff and Henikoff Amino acid substitution matrices from protein blocks. Proc. Natl. Acad. Sci. USA 89:10915-10919 (1989).
29. Hensley et al. Demonstration of cross-protective vaccine immunity against an emerging pathogenic Ebolavirus Species. PLOS Pathog. 6, e1000904 (2010).
30. Howell et al. Antibody Treatment of Ebola and Sudan Virus Infection via a Uniquely Exposed Epitope within the Glycoprotein Receptor-Binding Site. Cell Rep. 15, 1514-1526 (2016).
31. Jacob et al. Ebola virus disease. Nat. Rev. Dis. Primers 6:13 (2020).
32. Kanekiyo et al. Self-assembling influenza nanoparticle vaccines elicit broadly neutralizing H1N1 antibodies. Nature 499, 102-106 (2013).
33. Karlin and Altschul Applications and statistics for multiple high-scoring segments in molecular sequences. Proc. Nat'l. Acad. Sci. USA 90:5873-5787 (1993).
34. Kato et al. Multifaceted effects of antigen valency on B cell response composition and differentiation in vivo. Immunity 53, 548-563.e8 (2020).
35. Kimple et al. Overview of Affinity Tags for Protein Purification. Curr. Protoc. Protein Sci. 73: Unit-9.9. (2013).
36. Konduru et al. Ebolavirus Glycoprotein Fc Fusion Protein Protects Guinea Pigs against Lethal Challenge. PLOS ONE 11, e0162446 (2016).
37. Krieg et al. CpG motifs in bacterial DNA trigger direct B-cell activation. Nature 374, 546-549 (1995).
38. Kucharski et al. Effectiveness of ring vaccination as control strategy for ebola virus disease. Emerging Infect. Dis. 22, 105-108 (2016).
39. Ledgerwood et al. Chimpanzee Adenovirus Vector Ebola Vaccine. N Engl J Med. 2017 376 (10): 928-938 (2017).
40. Lee et al., Ebolavirus glycoprotein structure and mechanism of entry. Future Virol. 4, 621-635 (2009).
41. Lee et al. Structure of the Ebola virus glycoprotein bound to an antibody from a human survivor. Nature 454, 177-182 (2008).
42. Lehrer et al. Recombinant proteins of Zaire ebolavirus induce potent humoral and cellular immune responses and protect against live virus infection in mice. Vaccine 36, 3090-3100 (2018).
43. Lin et al. Broader neutralizing antibodies against H5N1 viruses using prime-boost immunization of hyperglycosylated hemagglutinin DNA and virus-like particles. PLOS ONE 7, e39075 (2012).
44. Liu et al. Intradermal immunization by Ebola virus GP subunit vaccines using microneedle patches protects mice against lethal EBOV challenge. Sci. Rep. 8, 11193 (2018).
45. Low et al. Optimisation of signal peptide for recombinant protein secretion in bacterial hosts.” Applied Microbiology and Biotechnology 97:3811-3826 (2013)
46. Luczkowiak et al. Broad Neutralizing Activity Against Ebolaviruses Lacking the Mucin-Like Domain in Convalescent Plasma Specimens From Patients With Ebola Virus Disease. J. Infect. Dis. 218, S574-S581 (2018).
47. Malvy et al. Ebola virus disease. Lancet 393:936-948 (2019).
48. Martinez et al. Impact of Ebola mucin-like domain on antiglycoprotein antibody responses induced by Ebola virus-like particles. J. Infect. Dis. 204 Suppl 3, S825-32 (2011).
49. Maruyama et al. Ebola virus can be effectively neutralized by antibody produced in natural human infection. J. Virol. 73, 6024-6030 (1999).
50. Marzi et al. Antibodies are necessary for rVSV/ZEBOV-GP-mediated protection against lethal Ebola virus challenge in nonhuman primates. Proc Natl Acad Sci USA 110, 1893-1898 (2013).
51. Matassov et al. Single-Dose Trivalent Vesiculo Vax Vaccine Protects Macaques from Lethal Ebolavirus and Marburgvirus Challenge. J Virol. 92 (3): e01190-17 (2018).
52. Meieret et al. The natural trimerization domain of T4 fibritin, dissociates into a monomeric A-state form containing a stable beta-hairpin: atomic details of trimer dissociation and local beta-hairpin stability from residual dipolar couplings. J. Mol. Biol. 344, 1051-1069 (2004).
53. Mire et al. Vesicular stomatitis virus-based vaccines protect nonhuman primates against Bundibugyo ebolavirus. PLOS Negl. Trop. Dis. 7, e2600 (2013).
54. Misasi et al. Structural and molecular basis for Ebola virus neutralization by protective human antibodies. Science 351, 1343-1346 (2016).
55. Moon et al. Enhancing humoral responses to a malaria antigen with nanoparticle vaccines that expand Tfh cells and promote germinal center induction. Proc Natl Acad Sci USA 109, 1080-1085 (2012).
56. Mulangu et al. A randomized, controlled trial of ebola virus disease therapeutics. N. Engl. J. Med. 381, 2293-2303 (2019).
57. Okemoto et al. A potent adjuvant monophosphoryl lipid A triggers various immune responses, but not secretion of IL-1beta or activation of caspase-1. J. Immunol. 176, 1203-1208 (2006).
58. Pantophlet et al. Hyperglycosylated mutants of human immunodeficiency virus (HIV) type 1 monomeric gp120 as novel antigens for HIV vaccine design. J. Virol. 77, 5889-5901 (2003).
59. Pascal et al. Development of Clinical-Stage Human Monoclonal Antibodies That Treat Advanced Ebola Virus Disease in Nonhuman Primates. J. Infect. Dis. 218, S612-S626 (2018).
60. Peng et al. Glycan shield of the ebolavirus envelope glycoprotein GP. Commun Biol. 5 (1): 785 (2022).
61. Powell et al. A Single Immunization with Spike-Functionalized Ferritin Vaccines Elicits Neutralizing Antibody Responses against SARS-COV-2 in Mice. ACS Cent. Sci. 7, 183-199 (2021).
62. Powell et al. Multimerization of Ebola GPΔmucin on protein nanoparticle vaccines has minimal effect on elicitation of neutralizing antibodies. Front Immunol. 13:942897 (2022 Aug. 24).
63. PREVAIL II Writing Group et al. A randomized, controlled trial of zmapp for ebola virus infection. N. Engl. J. Med. 375, 1448-1456 (2016).
64. Qiu, et al. Reversion of advanced Ebola virus disease in nonhuman primates with ZMapp. Nature 514, 47-53 (2014).
65. Reed et al. Key roles of adjuvants in modern vaccines. Nat. Med. 19, 1597-1608 (2013).
66. Regules et al. A recombinant vesicular stomatitis virus ebola vaccine. N. Engl. J. Med. 376, 330-341 (2017).
67. Rijal et al. Therapeutic Monoclonal Antibodies for Ebola Virus Infection Derived from Vaccinated Humans. Cell Rep. 27, 172-186.e7 (2019).
68. Schornberg et al. Role of endosomal cathepsins in entry mediated by the Ebola virus glycoprotein. J. Virol. 80, 4174-4178 (2006).
69. Sebastian et al. A Multi-Filovirus Vaccine Candidate: Co-Expression of Ebola, Sudan, and Marburg Antigens in a Single Vector. Vaccines (Basel) 8 (2): 241 (2020).
70. Sliepen et al. Presenting native-like HIV-1 envelope trimers on ferritin nanoparticles improves their immunogenicity. Retrovirology 12, 82 (2015).
71. Suder et al. The vesicular stomatitis virus-based Ebola virus vaccine: From concept to clinical trials. Hum. Vaccin. Immunother. 14, 2107-2113 (2018).
72. Wang et al. Ebola Viral Glycoprotein Bound to Its Endosomal Receptor Niemann-Pick C1. Cell 164, 258-268 (2016).
73. Wang et al. Prominent neutralizing antibody response targeting the ebolavirus glycoprotein subunit interface elicited by immunization. J. Virol. (2021) doi: 10.1128/JVI.01907-20.
74. Wec et al. Antibodies from a Human Survivor Define Sites of Vulnerability for Broad Protection against Ebolaviruses. Cell 169, 878-890.e15 (2017).
75. Wei et al. Progress of transposon vector system for production of recombinant therapeutic proteins in mammalian cells.” Front. Bioeng. Biotechnol. 10: article 879222 (2022) doi.org/10.3389/fbioe.2022.879222.
76. Weyer et al. Ebola virus disease: history, epidemiology and outbreaks. Curr. Infect. Dis. Rep. 17:480 (2015).
77. Woolsey et al. Current state of Ebola virus vaccines: A snapshot. PLOS Pathog. 17, e1010078 (2021).
78. Yassine et al. Hemagglutinin-stem nanoparticles generate heterosubtypic influenza protection. Nat. Med. 21, 1065-1070 (2015).
79. Zhang Self-assembly in the ferritin nano-cage protein super family. Int. J. Mol. Sci. 12:5406-5421 (2011).
80. Zhao et al. Immunization-Elicited Broadly Protective Antibody Reveals Ebolavirus Fusion Loop as a Site of Vulnerability. Cell 169, 891-904.e15 (2017).
81. Zhao et al. Toremifene interacts with and destabilizes the Ebola virus glycoprotein. Nature 535, 169-172 (2016).

It is understood that the examples and embodiments described in the present disclosure are for illustrative purposes only and that various modifications or changes in light thereof will be suggested to persons skilled in the art and are to be included within the spirit and purview of this application and scope of the appended claims. All publications, patents, and patent applications cited in the present disclosure are hereby incorporated by reference in their entirety for all purposes.

1. A fusion protein of an artificially modified amino acid sequence of an ebolavirus glycoprotein (GP) and an amino acid sequence of a ferritin subunit polypeptide, wherein the artificially modified amino acid sequence of the ebolavirus GP is a sequence with at least 90% sequence identity to SEQ ID NO:13, SEQ ID NO:14, or SEQ ID NO: 15.

2. (canceled)

3. The fusion protein of claim 1, wherein the artificially modified amino acid sequence of the ebolavirus GP comprises a deletion of the mucin-like domain.

4. The fusion protein of claim 1, wherein the artificially modified amino acid sequence of the ebolavirus GP comprises at least two artificial glycosylation sites.

5. (canceled)

6. The fusion protein of claim 4, wherein the artificial glycosylation sites are located in the glycan cap domain of the ebolavirus GP.

7. The fusion protein of claim 4, wherein the artificially modified amino acid sequence of the ebolavirus GP contains two or more amino acid substitutions at positions corresponding to positions 251, 280, 272, 274, 212, and 214 of SEQ ID NO:12, wherein the two or more amino acid substitutions result in the artificial glycosylation sites.

8. The fusion protein of claim 7, wherein the two or more amino acid substitutions are D251N, L280N, S272N, E274T, Q212N, and E212T.

9-14. (canceled)

15. The fusion protein of claim 1, wherein the amino acid sequence of the fusion protein is a sequence with at least 90% sequence identity to SEQ ID NO:9, SEQ ID NO:10, or SEQ ID NO:1.

16. A nanoparticle comprising an oligomer of the fusion protein of claim 1.

17-18. (canceled)

19. A nucleic acid encoding the fusion protein of claim 1.

20. The nucleic acid of claim 19, wherein the nucleic acid is DNA or RNA.

21. A vector comprising the nucleic acid of claim 19.

22. A cell comprising the nucleic acid of claim 19.

23. The cell of claim 22, wherein the cell is a mammalian cell.

24-26. (canceled)

27. A cell culture comprising a plurality of cells of claim 22.

28. An immunogenic composition comprising the fusion protein of claim 1.

29. An immunogenic composition comprising two or more different fusion proteins of claim 1.

30-31. (canceled)

32. A kit comprising the immunogenic composition of claim 28 and one or more of: a device for administering the immunogenic composition, and an excipient.

33. A method of inducing an immune response in a subject, the method comprising administering to the subject the immunogenic composition of claim 28.

34-37. (canceled)

38. A method of producing the fusion protein, comprising:
introducing into a cell the nucleic acid of claim 19;
incubating the cell under conditions allowing for expression of the fusion protein; and
isolating the fusion protein.

39-40. (canceled)

41. A method of producing a nanoparticle, comprising:
introducing into a cell the nucleic acid of claim 19;
incubating the cell under conditions allowing for expression of the fusion protein and self-assembly of the nanoparticle; and
isolating the nanoparticle.

42-43. (canceled)


SEQUENCE LISTING (29 sequences)

SEQ ID NO: 1 - protein, 676 aa, Zaire ebolavirus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 2 - protein, 676 aa, Bundibugyo ebolavirus
MVTSGILQLPRERFRKTSFFVWVIILFHKVFPIPLGVVHNNTLQVSDIDKLVCRDKLSSTSQLKSVGLNLEGNGVATDVPTATKRWGFRAGVPPKVVNYEAGEWAENCYNLDIKKADGSECLPEAPEGVRGFPRCRYVHKVSGTGPCPEGYAFHKEGAFFLYDRLASTIIYRSTTFSEGVVAFLILPETKKDFFQSPPLHEPANMTTDPSSYYHTVTLNYVADNFGTNMTNFLFQVDHLTYVQLEPRFTPQFLVQLNETIYTNGRRSNTTGTLIWKVNPTVDTGVGEWAFWENKKNFTKTLSSEELSVIFVPRAQDPGSNQKTKVTPTSFANNQTSKNHEDLVPEDPASVVQVRDLQRENTVPTPPPDTVPTTLIPDTMEEQTTSHYEPPNISRNHQERNNTAHPETLANNPPDNTTPSTPPQDGERTSSHTTPSPRPVPTSTIHPTTRETHIPTTMTTSHDTDSNRPNPIDISESTEPGPLTNTTRGAANLLTGSRRTRREITLRTQAKCNPNLHYWTTQDEGAAIGLAWIPYFGPAAEGIYTEGIMHNQNGLICGLRQLANETTQALQLFLRATTELRTFSILNRKAIDFLLQRWGGTCHILGPDCCIEPHDWTKNITDKIDQIIHDFIDKPLPDQTDNDNWWTGWRQWVPAGIGITGVIIAVIALLCICKFLL

SEQ ID NO: 3 - protein, 677 aa, Reston ebolavirus
MGSGYQLLQLPRERFRKTSFLVWVIILFQRAISMPLGIVTNSTLKATEIDQLVCRDKLSSTSQLKSVGLNLEGNGIATDVPSATKRWGFRSGVPPKVVSYEAGEWAENCYNLEIKKSDGSECLPLPPDGVRGFPRCRYVHKVQGTGPCPGDLAFHKNGAFFLYDRLASTVIYRGTTFTEGVVAFLILSEPKKHFWKATPAHEPVNTTDDSTSYYMTLTLSYEMSNFGGKESNTLFKVDNHTYVQLDRPHTPQFLVQLNETLRRNNRLSNSTGRLTWTLDPKIEPDVGEWAFWETKKNFSQQLHGENLHFQILSTHTNNSSDQSPAGTVQGKISYHPPTNNSELVPTDSPPVVSVLTAGRTEEMSTQGLTNGETITGFTANPMTTTIAPSPTMTSEVDNNVPSEQPNNTASIEDSPPSASNETIDHSEMNPIQGSNNSAQSPQTKTTPAPTASPMTQDPQETANSSKLGTSPGSAAEPSQPGFTINTVSKVADSLSPTRKQKRSVRQNTANKCNPDLHYWTAVDEGAAVGLAWIPYFGPAAEGIYIEGVMHNQNGLICGLRQLANETTQALQLFLRATTELRTYSLLNRKAIDFLLQRWGGTCRILGPSCCIEPHDWTKNITDEINQIKHDFIDNPLPDHGDDLNLWTGWRQWIPAGIGIIGVIIAIIALLCICKILC

SEQ ID NO: 4 - protein, 676 aa, Sudan ebolavirus
MGGLSLLQLPRDKFRKSSFFVWVIILFQKAFSMPLGVVTNSTLEVTEIDQLVCKDHLASTDQLKSVGLNLEGSGVSTDIPSATKRWGFRSGVPPKVVSYEAGEWAENCYNLEIKKPDGSECLPPPPDGVRGFPRCRYVHKAQGTGPCPGDYAFHKDGAFFLYDRLASTVIYRGVNFAEGVIAFLILAKPKETFLQSPPIREAVNYTENTSSYYATSYLEYEIENFGAQHSTTLFKIDNNTFVRLDRPHTPQFLFQLNDTIHLHQQLSNTTGRLIWTLDANINADIGEWAFWENKKNLSEQLRGEELSFEALSLNETEDDDAASSRITKGRISDRATRKYSDLVPKNSPGMVPLHIPEGETTLPSQNSTEGRRVGVNTQETITETAATIIGTNGNHMQISTIGIRPSSSQIPSSSPTTAPSPEAQTPTTHTSGPSVMATEEPTTPPGSSPGPTTEAPTLTTPENITTAVKTVLPQESTSNGLITSTVTGILGSLGLRKRSRRQTNTKATGKCNPNLHYWTAQEQHNAAGIAWIPYFGPGAEGIYTEGLMHNQNALVCGLRQLANETTQALQLFLRATTELRTYTILNRKAIDFLLRRWGGTCRILGPDCCIEPHDWTKNITDKINQIIHDFIDNPLPNQDNDDNWWTGWRQWIPAGIGITGIIIAIIALLCVCKLLC

SEQ ID NO: 5 - protein, 676 aa, Tai Forest ebolavirus
MGASGILQLPRERFRKTSFFVWVIILFHKVFSIPLGVVHNNTLQVSDIDKFVCRDKLSSTSQLKSVGLNLEGNGVATDVPTATKRWGFRAGVPPKVVNYEAGEWAENCYNLAIKKVDGSECLPEAPEGVRDFPRCRYVHKVSGTGPCPGGLAFHKEGAFFLYDRLASTIIYRGTTFAEGVIAFLILPKARKDFFQSPPLHEPANMTTDPSSYYHTTTINYVVDNFGTNTTEFLFQVDHLTYVQLEARFTPQFLVLLNETIYSDNRRSNTTGKLIWKINPTVDTSMGEWAFWENKKNFTKTLSSEELSFVPVPETQNQVLDTTATVSPPISAHNHAGEDHKELVSEDSTPVVQMQNIKGKDTMPTTVTGVPTTTPSPFPINARNTDHTKSFIGLEGPQEDHSTTQPAKTTSQPTNSTESTTLNPTSEPSSRGTGPSSPTVPNTTESHAELGKTTPTTLPEQHTAASAIPRAVHPDELSGPGFLTNTIRGVTNLLTGSRRKRRDVTPNTQPKCNPNLHYWTALDEGAAIGLAWIPYFGPAAEGIYTEGIMENQNGLICGLRQLANETTQALQLFLRATTELRTFSILNRKAIDFLLQRWGGTCHILGPDCCIEPQDWTKNITDKIDQIIHDFVDNNLPNQNDGSNWWTGWKQWVPAGIGITGVIIAIIALLCICKFML

SEQ ID NO: 6 - protein, 672 aa, Bombali ebolavirus
MILQVPEKRHQRTVLFIWLVILFQRAVSVPLGVIHNSTLQVSDIDKFVCHDKLTSTNQLRSIGLNLEGNGIATDVPSATKRWGFRAGVPPKVVGYEAGEWAENCYNLEIKKPDGSECLPMAPEGIRGFPRCRYVHKVSGTGSCESGFAFHKEGAFFLYDRLASTIIYRGTTFAEGVVAFIILPKAEKNFLQPPLTQGPTNTTNDPSSMYHSTTLEYETTRFGTNRSAFWFKVDNLTFVQLESRFTPQFLVELNETIYIEGKRSNTTGRLIWQVNSRVDTDVGEWAFWENKKNLKKSFPREELSLTAVPRAADSEHDAHPPEYTPGPDSNPTINDNTELVTEDPAHLVQLQRQGRKEILPTTIPQAIEREPPAAQHDNPRNSPTPPSPIESDITDSTQAEDLTHTDDPSTINSATEEPLPEVGETTQVPRDPEGPRRTQPTPPTGQPEQPSDNTMTPGQIHSESAAPMGERSIDGPGLLTNTLAGVARLITGAGRAKRESPEIRGAKCNPNLHYWTTHEESAAAGLAWIPYFGPAAEGIYTEGLMQNQNELICGLRQLANETTQALQLFLRSTTELRTFSILNRKAIDFLLQRWGGTCRILGPDCCIEPHDWTKNITDRIDQIIHDFVDKPLPDQSNNDNWWTGWRQWIPAGIGVVGVIAAFIALICICKIIC

SEQ ID NO: 7 - protein, 163 aa, Helicobacter pylori
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 8 - protein, 641 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGISGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 9 - protein, 641 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEINTTIGEWAFWETKKNLTRKIRSEELSFAGNITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGISGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 10 - protein, 641 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEINTTIGEWAFWETKKNLTRKIRNETLSFAGNITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGISGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 11 - protein, 641 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVNLTSRFTPQFLLQLNETIYTSGKRSNTTGKLIWKVNPEINTTIGEWAFWETKKNLTRKIRNETLSFAGNITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGISGGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 12 - protein, 444 aa, unidentified
  source 1..444: note = Ebolavirus
SIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGI

SEQ ID NO: 13 - protein, 447 aa, synthetic construct
SIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEINTTIGEWAFWETKKNLTRKIRSEELSFAGNITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGISGG

SEQ ID NO: 14 - protein, 444 aa, synthetic construct
SIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEINTTIGEWAFWETKKNLTRKIRNETLSFAGNITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGI

SEQ ID NO: 15 - protein, 448 aa, synthetic construct
QRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVNLTSRFTPQFLLQLNETIYTSGKRSNTTGKLIWKVNPEINTTIGEWAFWETKKNLTRKIRNETLSFAGNITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGI

SEQ ID NO: 16 - protein, 534 aa, unidentified
  source 1..534: note = Ebolavirus
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHG

SEQ ID NO: 17 - protein, 534 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEINTTIGEWAFWETKKNLTRKIRSEELSFAGNITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHG

SEQ ID NO: 18 - protein, 534 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEINTTIGEWAFWETKKNLTRKIRNETLSFAGNITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHG

SEQ ID NO: 19 - protein, 534 aa, synthetic construct
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVNLTSRFTPQFLLQLNETIYTSGKRSNTTGKLIWKVNPEINTTIGEWAFWETKKNLTRKIRNETLSFAGNITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIMKQIEDKIEEILSKIYHIENEIARIKKLIGEVASSSGLNDIFEAQKIEWHEAHHHHHHG

SEQ ID NO: 20 - protein, 31 aa, synthetic construct
MGVTGILQLPRDRFKRTSFFLWVIILFQRTF

SEQ ID NO: 21 - protein, 32 aa, synthetic construct
MKQIEDKIEEILSKIYHIENEIARIKKLIGEV

SEQ ID NO: 22 - protein, 15 aa, synthetic construct
GLNDIFEAQKIEWHE

SEQ ID NO: 23 - protein, 6 aa, synthetic construct
HHHHHH

SEQ ID NO: 24 - protein, 27 aa, synthetic construct
GYIPEAPRDGQAYVRKDGEWVLLSTFL

SEQ ID NO: 25 - protein, 10 aa, synthetic construct
EQKLISEEDL

SEQ ID NO: 26 - protein, 8 aa, synthetic construct
DYKDDDDK

SEQ ID NO: 27 - protein, 4 aa, synthetic construct
GSGG

SEQ ID NO: 28 - protein, 7 aa, synthetic construct
NGTGGSG

SEQ ID NO: 29 - protein, 5 aa, synthetic construct
GGGGS